SEQUENCE LISTING (166 sequences)

SEQ ID NO: 1 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1365 bp, synthetic construct
  source 1..1365: mol_type = other DNA
gaagtgcagctggtggaatctggcggcggactggtgcagcctggcggatctctgaagctgagctgtgccgccagcggcttcaccttcaacacctacgccatgaactgggtgcgccaggcctctggcaagggcctggaatgggtgggacggatcagaagcaagtacaacaattacgccacctactacgccgacagcgtgaaggaccggttcaccatcagccgggacgacagcaagagcaccctgtacctgcagatgaacagcctgaaaaccgaggacaccgccgtgtactactgcgtgcggcacggcaacttcggcaacagctatgtgtcttggtttgcctactggggccagggcaccctcgtgacagtgagctcagctagcaccaagggccccagcgtgttccccctggcgcccagcagcaagagcaccagcggcggcacagccgccctgggctgcctggtgaaggactacttccccgagccagtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 3 - protein, 215 aa, synthetic construct
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - DNA, 645 bp, synthetic construct
  source 1..645: mol_type = other DNA
caggctgtcgtgacccaggaacctagcctgaccgtgtctcctggcggaaccgtgaccctgacctgtagatctagcacaggcgccgtgaccaccagcaactacgccaattgggtgcagcagaagcccggccaggctcctagaggactgatcggcggcaccaacaagagagccccttggacccctgccagattcagcggctctctgctgggagataaggccgccctgacactgtctggcgcccagcctgaggatgaggccgagtacttttgcgccctgtggtacagcaacctgtgggtgttcggcggaggcaccaagctgaccgtgctgggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 5 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacacactgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 7 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPRVFLFPPKPKDTLMISRTPEVTCVVTGLRDEDPEVKFNWYVDGVEVHNAKTKPREEQYCGCYSVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgctgtcaccccagagtgtttctgttccctccaaagcctaaggacaccctgatgatcagcagaacccctgaagtgacctgtgtcgtgaccggcctgagagatgaggaccccgaagtgaagttcaattggtacgtggacggcgtggaagtgcacaacgccaagaccaagcctagagaggaacagtactgcggctgctacagcgtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacatgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 9 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPRVFLFPPKPKDTLYITREPEVTCVVTGLRDEDPEVKFNWYVDGVEVHNAKTKPREEQYCGCYSVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgctgtcaccccagagtgtttctgttccctccaaagcctaaggacacactgtacatcacccgcgagcctgaagtgacctgtgtcgtgacaggactgagagatgaggaccccgaagtgaagttcaattggtacgtggacggcgtggaagtgcacaacgccaagaccaagcctagagaggaacagtactgcggctgctacagcgtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacatgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 11 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLMISRTPEVTCVVVGLRHEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacaccctgatgatcagcagaacccctgaagtgacctgtgtggtcgtgggactgagacacgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 13 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLYITREPEVTCVVVGLRHEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacacactgtacatcacccgcgagcctgaagtgacatgtgtggttgtgggactgagacacgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 15 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLMISRTPEVTCVVTGLRDEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacaccctgatgatcagcagaacccctgaagtgacctgtgtcgtgaccggcctgagagatgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacatgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 17 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLYITREPEVTCVVTGLRDEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacacactgtacatcacccgcgagcctgaagtgacctgtgtcgtgacaggactgagagatgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacatgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 19 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLMISRTPEVTCVVVDVSHEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 21 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCHPSVFLFPPKPKDTLYITREPEVTCVVVDVSHEDPEVKFNWYVDGVEVHNAKTKPREEQYCGCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgtcacccttccgtgtttctgttccctccaaagcctaaggacacactgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtactgcggctgctacagagtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 23 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCGPSVFLFPPKPKDTLMISRTPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtattgcagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 25 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCGPSVFLFPPKPKDTLYITREPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgcggccctagcgttttcctgtttcctccaaagcctaaggacaccctctacatcacccgcgagcctgaagtgacatgtgtggtggtggatgtgtcccacgaggaccccgaagtgaagttcaattggtacgtggacggcgtggaagtgcacaacgccaagaccaagcctagagaggaacagtattgcagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 27 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 29 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgttgcggccctagcgttttcctgtttcctccaaagcctaaggacaccctctacatcacccgcgagcctgaagtgacatgtgtggtggtggatgtgtcccacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 31 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtattgcagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 33 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacacactgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtattgcagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 35 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgctctgcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 37 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgtggcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 39 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLCGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgctgtgcggccctagcgttttcctgtttcctccaaagcctaaggacaccctctacatcacccgcgagcctgaagtgacatgtgtggtggtggatgtgtcccacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 41 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgtggcggccctagcgttttcctgtttcctccaaagcctaaggacaccctctacatcacccgcgagcctgaagtgacatgtgtggtggtggatgtgtcccacgaggaccccgaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 43 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtataacagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 45 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtattgcagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 47 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacacactgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtataacagctgctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 49 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagaactgctcggcggaccctccgtgtttctgttccctccaaagcctaaggacacactgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtattgcagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 51 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCGGPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgtggcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtattgcagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 53 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCGGPSVFLFPPKPKDTLYITREPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1365 bp, synthetic construct
  source 1..1365: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttccactggctcctagcagcaagtctacctctggtggaacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagctctagcctgggcacccagacctacatctgcaacgtgaaccacaagcctagcaacaccaaggtcgacaagagagtggaacccaagagctgcgacaagacccacacctgtcctccatgtcctgctccagagctgtgtggcggccctagcgttttcctgtttcctccaaagcctaaggacaccctctacatcacccgcgagcctgaagtgacatgtgtggtggtggatgtgtcccacgaggaccccgaagtgaagttcaattggtacgtggacggcgtggaagtgcacaacgccaagaccaagcctagagaggaacagtattgcagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaag

SEQ ID NO: 55 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 1365 bp, synthetic construct
  source 1..1365: mol_type = other DNA
gaagtgcagctggtggaatctggcggcggactggtgcagcctggcggatctctgaagctgagctgtgccgccagcggcttcaccttcaacacctacgccatgaactgggtgcgccaggcctctggcaagggcctggaatgggtgggacggatcagaagcaagtacaacaattacgccacctactacgccgacagcgtgaaggaccggttcaccatcagccgggacgacagcaagagcaccctgtacctgcagatgaacagcctgaaaaccgaggacaccgccgtgtactactgcgtgcggcacggcaacttcggcaacagctatgtgtcttggtttgcctactggggccagggcaccctcgtgacagtgagctcagctagcaccaagggccccagcgtgttccccctggcgccctctagcaagagcaccagcggaggaacagccgccctgggctgcctggtcaaggactactttcccgagcccgtgaccgtgtcctggaacagcggagcactgaccagcggcgtccacacctttccagccgtgctccagagcagcggcctgtactctctgagcagcgtggtgaccgtgcctagcagcagcctgggcacccagacctacatctgtaacgtgaaccacaagcccagcaacaccaaggtggacaagagagtggaacccaagtcttgcgacaagacccacacctgccctccctgtccagcccctgaactgctgggaggccctagcgtgttcctgttccccccaaagcccaaggacaccctgatgatcagccggacccccgaagtgacctgtgtggtggtggccgtgtctcacgaggaccctgaagtgaagtttaattggtacgtggacggcgtggaagtgcacaacgccaagaccaagcccagagaggaacagtacgccagcacctaccgggtggtgtccgtgctgacagtgctgcaccaggactggctgaacggcaaagagtacaagtgcaaggtgtccaacaaggccctggccgctcccatcgagaaaaccatcagcaaggccaagggccagccccgcgaaccccaggtgtacacactgccccctagcagggacgagctgaccaagaaccaggtgtccctgacctgcctcgtgaagggcttctacccctccgatatcgccgtggaatgggagagcaacggccagcccgagaacaactacaagaccaccccccctgtgctggactccgacggctcattcttcctgtacagcaagctgaccgtggacaagtcccggtggcagcagggcaacgtgttcagctgctccgtgatgcacgaggccctgcacaaccactacacccagaagtccctgagcctgagccccggcaaa

SEQ ID NO: 57 - protein, 455 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 1365 bp, synthetic construct
  source 1..1365: mol_type = other DNA
gaagtgcagctggtggaatctggcggcggactggtgcagcctggcggatctctgaagctgagctgtgccgccagcggcttcaccttcaacacctacgccatgaactgggtgcgccaggcctctggcaagggcctggaatgggtgggacggatcagaagcaagtacaacaattacgccacctactacgccgacagcgtgaaggaccggttcaccatcagccgggacgacagcaagagcaccctgtacctgcagatgaacagcctgaaaaccgaggacaccgccgtgtactactgcgtgcggcacggcaacttcggcaacagctatgtgtcttggtttgcctactggggccagggcaccctcgtgacagtgagctcagctagcaccaagggccccagcgtgttccccctggcgccctctagcaagagcaccagcggaggaacagccgccctgggctgcctggtcaaggactactttcccgagcccgtgaccgtgtcctggaacagcggagcactgaccagcggcgtccacacctttccagccgtgctccagagcagcggcctgtactctctgagcagcgtggtgaccgtgcctagcagcagcctgggcacccagacctacatctgtaacgtgaaccacaagcccagcaacaccaaggtggacaagagagtggaacccaagtcttgcgacaagacccacacctgccctccctgtccagcccctgaactgctgggaggccctagcgtgttcctgttccccccaaagcccaaggacaccctgtacatcacccgggagcccgaagtgacctgtgtggtggtggccgtgtctcacgaggaccctgaagtgaagtttaattggtacgtggacggcgtggaagtgcacaacgccaagaccaagcccagagaggaacagtacgccagcacctaccgggtggtgtccgtgctgacagtgctgcaccaggactggctgaacggcaaagagtacaagtgcaaggtgtccaacaaggccctggccgctcccatcgagaaaaccatcagcaaggccaagggccagccccgcgaaccccaggtgtacacactgccccctagcagggacgagctgaccaagaaccaggtgtccctgacctgcctcgtgaagggcttctacccctccgatatcgccgtggaatgggagagcaacggccagcccgagaacaactacaagaccaccccccctgtgctggactccgacggctcattcttcctgtacagcaagctgaccgtggacaagtcccggtggcagcagggcaacgtgttcagctgctccgtgatgcacgaggccctgcacaaccactacacccagaagtccctgagcctgagccccggcaaa

SEQ ID NO: 59 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - DNA, 1458 bp, synthetic construct
  source 1..1458: mol_type = other DNA
gaagttcagcttgttgaatctggcggcggactggtgcaacctggcggatctctgaaactgtcttgtgccgcctccggcttcaccttcaatacctacgccatgaactgggttcgacaagcctccggcaaaggactggaatgggtcggacggatcagaagcaagtacaacaactacgccacctactacgccgactccgtgaaggacagattcaccatcagccgggacgactccaagagcaccctgtacctccagatgaactccctgaaaaccgaggacacagccgtctattattgcgtgcggcacggcaacttcggcaacagctatgtgtcttggtttgcctactggggacagggaaccctcgtgaccgtttcttcaggcggcggtggtagtggcggtggtggtagcggaggcggtggatcaggtggcggcggttctcaagctgtggtcacacaagagcccagcctgacagtttctcctggcggaaccgtgacactgacctgtagatctagcaccggcgcagtgaccaccagcaattacgctaactgggtgcagcagaagcccggccaagctcctagaggactgatcggaggcacaaacaagagagccccttggacaccagccagattttctggctctctgctgggcgataaggccgctcttacactgtctggcgcacagcctgaagatgaggccgagtacttttgcgccctgtggtacagcaacctgtgggtgttcggcggaggaacaaagctgacagttcttggaggcggcggaagcgacaagacccacacatgtcctccatgtcctgctccagaactgctcggcggaccctccgtgtttctgttccctccaaagccaaaggacaccctgatgatcagcagaacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgacagtgctgcaccaggactggctgaacggcaaagagtacaagtgcaaggtgtccaacaaggccctgcctgctcctatcgagaaaaccatcagcaaggccaagggccagcctagggaacctcaagtgtgcactctgcctccaagccgggaagagatgaccaagaatcaggtgtccctgagctgcgccgtgaagggcttttacccttccgatatcgccgtggaatgggagagcaatggccagccagagaacaactacaagaccacacctcctgtgctggacagcgacggctcattcttcctggtgtctaagctgaccgtggacaagagcagatggcagcagggcaacgtgttcagctgttctgtgatgcacgaggccctgcacaaccactacacacagaagtccctgtctctgagccccggcaaa

SEQ ID NO: 61 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - DNA, 1458 bp, synthetic construct
  source 1..1458: mol_type = other DNA
gaagttcagcttgttgaatctggcggcggactggtgcaacctggcggatctctgaaactgtcttgtgccgcctccggcttcaccttcaatacctacgccatgaactgggttcgacaagcctccggcaaaggactggaatgggtcggacggatcagaagcaagtacaacaactacgccacctactacgccgactccgtgaaggacagattcaccatcagccgggacgactccaagagcaccctgtacctccagatgaactccctgaaaaccgaggacacagccgtctattattgcgtgcggcacggcaacttcggcaacagctatgtgtcttggtttgcctactggggacagggaaccctcgtgaccgtttcttcaggcggcggtggtagtggcggtggtggtagcggaggcggtggatcaggtggcggcggttctcaagctgtggtcacacaagagcccagcctgacagtttctcctggcggaaccgtgacactgacctgtagatctagcaccggcgcagtgaccaccagcaattacgctaactgggtgcagcagaagcccggccaagctcctagaggactgatcggaggcacaaacaagagagccccttggacaccagccagattttctggctctctgctgggcgataaggccgctcttacactgtctggcgcacagcctgaagatgaggccgagtacttttgcgccctgtggtacagcaacctgtgggtgttcggcggaggaacaaagctgacagttcttggaggcggcggaagcgacaagacccacacatgtcctccatgtcctgctccagaactgctcggcggaccctccgtgtttctgttccctccaaagccaaaggacaccctgatgatcagcagaacccctgaagtgacctgtgtggtggtggccgtgtctcacgaggacccagaagtgaagttcaattggtacgtggacggcgtggaagtgcacaacgccaagaccaagcctagagaggaacagtacgccagcacctacagagtggtgtctgtgctgacagtgctgcaccaggactggctgaacggcaaagagtacaagtgcaaggtgtccaacaaggccctggccgctcctatcgagaaaaccatctctaaggccaagggccagcctcgggaacctcaagtgtgcactcttccacctagccgggaagagatgaccaagaaccaggtgtcactgagctgcgccgtgaagggcttctacccttctgatatcgccgtggaatgggagagcaacggccagccagagaacaactacaagaccacacctcctgtgctggacagcgacggctcattcttcctggtgtctaagctgaccgtggacaagagcagatggcagcagggcaacgtgttcagctgttctgtgatgcacgaggccctgcacaaccactacacacagaagtccctgtctctgagccccggcaaa

SEQ ID NO: 63 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELCCHPRVFLFPPKPKDTLMISRTPEVTCVVTGLRDEDPEVKFNWYVDGVEVHNAKTKPREEQYCGCYSVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgtgttgtcatccgcgtgtttttctgtttccgccgaaaccgaaagataccctgatgattagtcgtaccccggaagttacctgtgttgttacaggtctgcgtgatgaagacccggaagtgaaatttaattggtatgtggatggcgtggaagtgcataatgccaaaaccaaaccgcgcgaagaacagtattgtggctgctatagcgttgttagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 65 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELCCHPSVFLFPPKPKDTLMISRTPEVTCVVVGLRHEDPEVKFNWYVDGVEVHNAKTKPREEQYCGCYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgtgttgtcatccgagcgtttttctgtttccgccgaaaccgaaagataccctgatgattagtcgtaccccggaagttacctgcgttgttgttggtctgcgtcatgaagacccggaagtgaaatttaattggtatgtggatggcgtggaagtgcataatgccaaaaccaaaccgcgcgaagaacagtattgtggttgctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 67 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELCCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 68 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgtgttgtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 69 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVDVSHEDPEVKFNWYVDGVEVHNAKTKPREEQYCSCYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgctgggtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtattgtagctgctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 71 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgctgtgtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 73 - protein, 486 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - DNA, 1458 bp, synthetic construct
  source 1..1458: mol_type = other DNA
gaagttcagctggttgaaagtggtggtggtctggttcagccgggtggtagtctgaaactgagttgtgcagcaagcggctttacctttaatacctatgccatgaattgggtgcgtcaggcaagtggtaaaggtctggaatgggttggtcgtattcgcagcaaatataataattatgccacctattatgccgatagcgtgaaagatcgctttaccattagccgtgatgatagcaaaagcaccctgtatttacagatgaatagcctgaaaaccgaagataccgccgtgtattattgcgtgcgtcatggcaattttggcaatagctatgtgagctggtttgcctattggggtcagggtacactggttacagttagcagtggtggtggtggttcaggtggtggtggtagtggtggtggtggttcaggtggtggtggtagtcaggcagttgttacacaggaaccgagtctgacagttagtccgggtggtacagttaccctgacctgtcgtagtagtacaggtgcagttaccaccagcaattatgccaattgggttcagcagaaaccgggtcaggcaccgcgtggtctgattggtggtacaaataaacgtgcaccgtggacaccggcacgttttagtggtagtctgctgggtgataaagcagcactgacactgagtggtgcacagccggaagatgaagccgaatatttttgtgccctgtggtatagcaatctgtgggtttttggtggtggcaccaaactgacagttctgggtggtggtggtagcgataaaacccatacatgtccgccgtgtccggcaccggaactgtgtggtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttgtacactgccgccgagtcgtgaagaaatgaccaaaaatcaggttagcctgagctgcgccgtgaaaggtttttatccgagcgatattgccgtggaatgggaaagtaatggccagccggaaaataattataaaaccacaccgccggtgctggatagtgatggtagcttttttctggtgagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 75 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 681 bp, synthetic construct
  source 1..681: mol_type = other DNA
gataagacacacacatgtcctccatgtcctgctccagagctgctcggcggaccttctgttttcctgtttccacctaagccaaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacaccctgcctccatgccgggaagagatgaccaagaatcaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaaccactacacccagaagtctctgtctctgagccccggcaaa

SEQ ID NO: 77 - protein, 227 aa, synthetic construct
DKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - DNA, 681 bp, synthetic construct
  source 1..681: mol_type = other DNA
gataagacacacacatgtcctccatgtcctgctccagagctgctcggcggaccttctgttttcctgtttccacctaagccaaaggacaccctgatgatcagcagaacccctgaagtgacctgtgtggtggtggccgtgtctcacgaagatcccgaagtgaagttcaattggtacgtggacggcgtggaagtgcacaacgccaagaccaagcctagagaggaacagtacgccagcacctatagagtggtgtccgtgctgacagtgctgcaccaggactggctgaacggcaaagagtacaagtgcaaggtgtccaacaaggccctggccgctcctatcgagaaaaccatctctaaggccaagggccagcctcgggaaccacaggtttacaccctgcctccatgccgggaagagatgaccaagaatcaggtgtccctgtggtgcctggtcaagggcttctacccttccgatatcgccgtggaatgggagagcaatggccagcctgagaacaactacaagaccacacctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaaa

SEQ ID NO: 79 - protein, 227 aa, synthetic construct
DKTHTCPPCPAPELCCHPRVFLFPPKPKDTLMISRTPEVTCVVTGLRDEDPEVKFNWYVDGVEVHNAKTKPREEQYCGCYSVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 681 bp, synthetic construct
  source 1..681: mol_type = other DNA
gataaaacacatacatgtccgccgtgtccggcaccggaactgtgttgtcatccgcgtgtttttctgtttccgccgaaaccgaaagataccctgatgattagtcgtaccccggaagttacctgtgttgttacaggtctgcgtgatgaagacccggaagtgaaatttaattggtatgtggatggcgtggaagtgcataatgccaaaaccaaaccgcgcgaagaacagtattgtggctgctatagcgttgttagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 81 - protein, 227 aa, synthetic construct
DKTHTCPPCPAPELCCHPSVFLFPPKPKDTLMISRTPEVTCVVVGLRHEDPEVKFNWYVDGVEVHNAKTKPREEQYCGCYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - DNA, 681 bp, synthetic construct
  source 1..681: mol_type = other DNA
gataaaacacatacatgtccgccgtgtccggcaccggaactgtgttgtcatccgagcgtttttctgtttccgccgaaaccgaaagataccctgatgattagtcgtaccccggaagttacctgcgttgttgttggtctgcgtcatgaagacccggaagtgaaatttaattggtatgtggatggcgtggaagtgcataatgccaaaaccaaaccgcgcgaagaacagtattgtggttgctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 83 - protein, 227 aa, synthetic construct
DKTHTCPPCPAPELCCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 681 bp, synthetic construct
  source 1..681: mol_type = other DNA
gataaaacacatacatgtccgccgtgtccggcaccggaactgtgttgtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 85 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 681 bp, synthetic construct
  source 1..681: mol_type = other DNA
gataaaacacatacatgtccgccgtgtccggcaccggaactgctgggtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtattgtagctgctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 87 - protein, 227 aa, synthetic construct
DKTHTCPPCPAPELLCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 681 bp, synthetic construct
  source 1..681: mol_type = other DNA
gataaaacacatacatgtccgccgtgtccggcaccggaactgctgtgtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 89 - protein, 227 aa, synthetic construct
DKTHTCPPCPAPELCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 681 bp, synthetic construct
  source 1..681: mol_type = other DNA
gataaaacacatacatgtccgccgtgtccggcaccggaactgtgtggtggtccgagtgtttttctgtttccgccgaaaccgaaagataccctgatgattagtcgtaccccggaagttacctgcgttgttgttgatgtgagccatgaagacccggaagtgaaatttaattggtatgtggatggcgtggaagtgcataatgccaaaaccaaaccgcgcgaagaacagtataatagcacctatcgtgttgtgagcgttctgaccgttctgcatcaggattggctgaatggcaaagaatataaatgcaaagtgagcaataaagccctgccggcaccgattgaaaaaaccattagcaaagcaaaaggccagccgcgtgaaccgcaggtttatacactgccgccgtgtcgtgaagaaatgaccaaaaatcaggttagcctgtggtgcctggtgaaaggtttttatccgagcgatattgccgtggaatgggaaagtaatggccagccggaaaataattataaaaccacaccgccggtgctggatagtgatggtagcttttttctgtatagcaaactgaccgtggataaaagccgttggcagcagggtaatgtgtttagctgtagcgttatgcatgaagccctgcataatcattatacccagaaaagcctgagcctgagtccgggtaaa

SEQ ID NO: 91 - protein, 107 aa, synthetic construct [sequence omitted]

SEQ ID NO: 92 - DNA, 321 bp, synthetic construct
  source 1..321: mol_type = other DNA
agaacagtggccgctccgagcgtgttcatctttccaccaagcgacgagcagctgaaaagcggcacagcctctgtcgtgtgcctgctgaacaacttctaccccagagaagccaaggtgcagtggaaggtggacaatgccctccagtccggcaatagccaagagagcgtgaccgagcaggacagcaaggatagcacatacagcctgagcagcacactgaccctgagcaaggccgactacgagaagcacaaagtgtacgcctgcgaagtgacacaccagggcctgtctagccctgtgaccaagagcttcaacagaggcgagtgc

SEQ ID NO: 93 - protein, 227 aa, synthetic construct
DKTHTCPPCPAPELCCHPRVFLFPPKPKDTLMISRTPEVTCVVTGLRDEDPEVKFNWYVDGVEVHNAKTKPREEQYCGCYSVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - DNA, 681 bp, synthetic construct
  source 1..681: mol_type = other DNA
gataagacccacacctgtcctccatgtcctgctccagagctgtgctgtcaccccagagtgtttctgttccctccaaagcctaaggacaccctgatgatcagcagaacccctgaagtgacctgtgtcgtgaccggcctgagagatgaggaccccgaagtgaagttcaattggtacgtggacggcgtggaagtgcacaacgccaagaccaagcctagagaggaacagtactgcggctgctacagcgtggtgtctgtgctgacagtgctgcaccaggactggctgaacggcaaagagtacaagtgcaaggtgtccaacaaggccctgcctgctcctatcgagaaaaccatcagcaaggccaagggccagccaagagaaccccaggtttacaccctgcctccaagccgggaagagatgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgaccgtggacaagagcagatggcagcagggcaacgtgttcagctgttctgtgatgcacgaggccctgcacaaccactacacccagaagtctctgtctctgagccccggcaaa

SEQ ID NO: 95 - protein, 227 aa, synthetic construct
DKTHTCPPCPAPELCCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - DNA, 681 bp, synthetic construct
  source 1..681: mol_type = other DNA
gataagacccacacctgtcctccatgtcctgctccagagctgtgttgcggcccctccgttttcctgtttccacctaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacaccctgcctccaagccgggaagagatgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaaccactacacccagaagtctctgtctctgagccccggcaaa

SEQ ID NO: 97 - protein, 227 aa, synthetic construct
DKTHTCPPCPAPELLCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 681 bp, synthetic construct
  source 1..681: mol_type = other DNA
gataagacccacacctgtcctccatgtcctgctccagaactgctgtgcggcccctccgttttcctgtttccacctaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacaccctgcctccaagccgggaagagatgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaaccactacacccagaagtctctgtctctgagccccggcaaa

SEQ ID NO: 99 - protein, 227 aa, synthetic construct
DKTHTCPPCPAPELCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 681 bp, synthetic construct
  source 1..681: mol_type = other DNA
gataagacccacacctgtcctccatgtcctgctccagaactgtgtggcggccctagcgttttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagccaagagaaccccaggtttacaccctgcctccaagccgggaagagatgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaaccactacacccagaagtctctgtctctgagccccggcaaa

SEQ ID NO: 101 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 102 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 103 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 104 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctccatgtcctgctccagaatttctcggcggacccagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 105 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCHPRVFLFPPKPKDTLMISRTPEVTCVVTGLRDEDPEVQFNWYVDGVEVHNAKTKPREEQFCGCYSVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 106 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttctgctgtcaccccagagtgtttctgttccctccaaagcctaaggacaccctgatgatcagcagaacccctgaagtgacctgtgtcgtgaccggcctgagagatgaagatcccgaggtgcagttcaattggtacgtggacggcgtggaagtgcacaacgccaagaccaagcctagagaggaacagttctgcggctgctacagcgtggtgtctgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 107 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCHPSVFLFPPKPKDTLMISRTPEVTCVVVGLRQEDPEVQFNWYVDGVEVHNAKTKPREEQFCGCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 108 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttctgttgtcacccttccgtgtttctgttccctccaaagcctaaggacaccctgatgatcagcagaacccctgaagtgacctgtgtggtcgtgggccttagacaagaggaccccgaggtgcagttcaattggtacgtggacggcgtggaagtgcacaacgccaagaccaagcctagagaggaacagttctgcggctgctacagagtggtgtctgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 109 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCGPSVFLFPPKPKDTLMISRTPEVTCVVVDVSQEDPEVQFNWYVDGVEVHNAKTKPREEQFCSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 110 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttttgttgcggccctagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttctgctcctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 111 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 112 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttttgttgcggccctagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 113 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFCSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 114 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttctgctcctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 115 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLCGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 116 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctgtgcggccctagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 117 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 118 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaattttgtggcggccctagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 119 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 120 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttcaacagctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 121 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 122 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttctgctccacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 123 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 124 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaattttgtggcggccctagcgtgttcctgtttcctccaaagcctaaggacaccctgatgatcagcagaacccctgaagtgacctgcgtggtggtggatgtgtctcaagaggaccccgaggtgcagttcaattggtacgtggacggcgtggaagtgcacaacgccaagaccaagcctagagaggaacagttctgctccacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 125 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 126 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 127 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 128 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctccatgtcctgctccagaatttctcggcggacccagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 129 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCHPRVFLFPPKPKDTLYITREPEVTCVVTGLRDEDPEVQFNWYVDGVEVHNAKTKPREEQFCGCYSVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 130 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttctgctgtcaccccagagtgtttctgttccctccaaagcctaaggacacactgtacatcacccgcgagcctgaagtgacctgtgtcgtgacaggactgagagatgaggaccccgaggtgcagttcaattggtacgtggacggcgtggaagtgcacaacgccaagaccaagcctagagaggaacagttctgcggctgctacagcgtggtgtctgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 131 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCHPSVFLFPPKPKDTLYITREPEVTCVVVGLRQEDPEVQFNWYVDGVEVHNAKTKPREEQFCGCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 132 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttctgttgtcacccttccgtgtttctgttccctccaaagcctaaggacacactgtacatcacccgcgagcctgaagtgacatgtgtggtcgtgggactgagacaagaggaccccgaggtgcagttcaattggtacgtggacggcgtggaagtgcacaacgccaagaccaagcctagagaggaacagttctgcggctgctacagagtggtgtctgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 133 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCGPSVFLFPPKPKDTLYITREPEVTCVVVDVSQEDPEVQFNWYVDGVEVHNAKTKPREEQFCSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 134 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttttgttgcggccctagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttctgctcctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 135 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCCGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 136 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgagttttgttgcggccctagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 137 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFCSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 138 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttctgctcctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 139 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLCGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 140 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctgtgcggccctagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 141 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 142 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaattttgtggcggccctagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagcacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 143 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSCYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 144 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttcaacagctgctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 145 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 146 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaatttctcggcggacccagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttctgctccacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 147 - protein, 450 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFCGGPSVFLFPPKPKDTLYITREPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 148 - DNA, 1350 bp, synthetic construct
  source 1..1350: mol_type = other DNA
gaagtgcaactggttgaatctggcggaggactggttcagcctggcggatctctgaaactgtcttgtgccgccagcggcttcaccttcaacacctacgccatgaactgggtccgacaggcctctggcaaaggcctggaatgggtcggacggatcagaagcaagtacaacaattacgccacctactacgccgacagcgtgaaggacagattcaccatcagccgggacgacagcaagagcaccctgtacctccagatgaacagcctgaaaaccgaggataccgccgtgtactattgcgtgcggcacggcaacttcggcaacagctatgtgtcttggtttgcctactggggccagggcacactggttacagtgtctagcgcctctacaaagggcccctccgtttttcctctggctccttgttccagaagcaccagcgaatctacagccgctctgggctgcctggtcaaggattactttcctgagcctgtgaccgtgtcctggaatagcggagcactgacaagcggcgtgcacacatttccagccgtgctgcaaagcagcggcctgtactctctgtctagcgtggtcacagtgcctagcagcagcctgggcaccaagacctacacctgtaacgtggaccacaagcctagcaataccaaggtggacaagcgcgtggaatctaagtacggccctccttgtcctagctgccctgctcctgaattttgtggcggccctagcgtgttcctgtttcctccaaagcctaaggacacactgtacatcacccgcgagcctgaagtgacctgtgtggtggtggatgtgtcccaagaggaccccgaggtgcagttcaattggtacgtggacggcgtggaagtgcacaacgccaagaccaagcctagagaggaacagttctgctccacctacagagtggtgtccgtgctgacagtgctgcaccaggactggctgaacggcaaagagtacaagtgcaaggtgtccaacaagggcctgccaagcagcatcgagaaaaccatcagcaaggccaagggccagcctagggaacctcaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggacagcgacggcagcttttttctgtattctcggctgaccgtggacaagagcagatggcaagagggcaacgtgttcagctgctctgtgatgcacgaggccctgcacaaccactacacccagaagtctctgtctctgagcctg

SEQ ID NO: 149 - protein, 107 aa, synthetic construct [sequence omitted]

SEQ ID NO: 150 - DNA, 321 bp, synthetic construct
  source 1..321: mol_type = other DNA
cggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 151 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgtgctgcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 153 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYCSCYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtactgcagctgctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 155 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLCGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgtgcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 157 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELCGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgtgcggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 159 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSCYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagctgctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 161 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtactgcagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 163 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggccgtgagccacgaggaccccgaggtgaagttcaactggtacgtggacggcgtggaggtgcacaacgccaagaccaagccccgggaggagcagtacgccagcacctaccgggtggtgagcgtgctgaccgtgctgcaccaggactggctgaacggcaaggagtacaagtgcaaggtgagcaacaaggccctggccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 165 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - DNA, 990 bp, synthetic construct
  source 1..990: mol_type = other DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

(19) United States
(12) Patent Application Publication
(10) Pub. No.: US 2025/0011428 A1
(43) Pub. Date: Jan. 9, 2025

(54) ENGINEERED FC VARIANTS

(71) Applicant: Novartis AG, Basel (CH)

(72) Inventors: Justine Celine Patricia GUYOT, Zimmersheim (FR); Sebastien IRIGARAY, Zimmersheim (FR); Darko SKEGRO, Riehen (CH)

(21) Appl. No.: 18/705,653

(22) PCT Filed: Oct. 27, 2022
(86) PCT No.: PCT/IB2022/060324

§ 371 Date: Apr. 29, 2024

Related U.S. Application Data
(60) Provisional application No. 63/272,955, filed on Oct. 28, 2021.

(51) Int. Cl. C07K 16/28

(52) U.S. Cl. CPC C07K 16/2809; C07K 2317/31; C07K 2317/524; C07K 2317/71; C07K 2317/92; C07K 2317/94

(57) ABSTRACT

The present invention describes engineered immunoglobulin IgG Fc regions by transferring structural elements, several CH2 inter-chain disulfide bonds, from IgA to IgG immunoglobulin. The disclosed Fc variants created thereof exhibit marked reductions or complete abrogation of interaction of the engineered Fc with FcγR and C1q while retaining natural ability to interact with FcRn at acidic pH. Silenced Fc molecules disclosed are in comparable expression and purification yields and improved or maintained thermostability compared to wild-type Fc, thereby limiting the propensity for aggregation. In addition, the disclosed Fc variant silencing mutations are capable of reducing or compensating destabilizing effects of other half-life extending or chain pairing facilitating Fc mutations.

### FIELD OF THE INVENTION

The invention relates to molecules, such as engineered IgG immunoglobulins, that comprises Fc variants obtained via transferring structural elements (e.g. CH2 inter-chain disulfide bonds) from IgA to IgG immunoglobulin, which exhibit highly diminished or fully eliminated Fc effector functions, whilst maintaining highly stable physicochemical properties. These silenced Fc variants are particularly advantageous when used in combination with various other, often destabilizing substitutions in the Fc CH2 domain, such as half-life extending or chain pairing mutations. The molecules according to the instant invention are useful for the development of therapeutics, with superior properties, such as enhanced stability, developability and/or half-life.

### BACKGROUND OF THE INVENTION

Immunoglobulins (e.g., antibodies) can be separated functionally into variable domains that binds antigens and constant domains that specify effector functions such as activation of complement or binding to Fc receptors. There are five main classes of heavy chain constant domains, each class defining the immunoglobulin isotype (IgM, IgG, IgA, IgD, and IgE). IgG can be split into four subclasses, IgG1, IgG2, IgG3, and IgG4; and IgA similarly into two subclasses IgA1 and IgA2. Despite the fact constant domains of immunoglobulin classes G (IgG) and A (IgA) have different amino acid sequences, they exhibit strong structural homology. In fact, both classes are made of immunoglobulin like domains and share very similar protein folding. However, structural differences remain, particularly within CH2 domain of the crystallizable fragment.

The effector functions attributable to the Fc region of an immunoglobulin (e.g., an antibody) vary with the class and subclass of immunoglobulin (e.g., antibody) and include binding of the immunoglobulin (e.g., antibody) via the Fc region to a specific Fc receptor on a cell which triggers various biological responses. These receptors are expressed in a variety of immune cells, for example monocytes, macrophages, neutrophils, dendritic cells, eosinophils, mast cells, platelets, B cells, large granular lymphocytes, Langerhans' cells, natural killer (NK) cells, and T cells. Formation of the Fc/Fc receptor complex (e.g. FcγR complex) recruits these effector cells to sites of bound antigen, typically resulting in signaling events within the cells and important subsequent immune responses such as release of inflammation mediators, B cell activation, endocytosis, phagocytosis, and/or cytotoxic attack. In addition, an overlapping site on the Fc region of the molecule also controls the activation of a cell independent cytotoxic function mediated by complement, otherwise known as complement dependent cytotoxicity (CDC).

In some instances, it may be advantageous to decrease or even to fully eliminate the effector functions. This is particularly true for those antibodies designed to deliver a drug (e.g., toxins and isotopes) to the target cell where the Fc/Fc receptor mediated effector functions bring healthy immune cells into the proximity of the deadly payload, resulting in depletion of normal lymphoid tissue along with the target cells (Hutchins, et al., 1995; White, et al., 2001). In addition, for cases where mAbs are intended to engage cell surface receptors and prevent receptor-ligand interactions (for example antagonists, e.g. antagonists of cytokines), it may be desirable to reduce or eliminate effector function for example to prevent target cell death or unwanted cytokine secretion. In these cases, the use of antibodies that poorly recruit complement or effector cells would be of a tremendous benefit. The need for reducing or eliminating effector function was recognized with the first approved mAb, the anti-CD3 mAb, muromonab-CD3, which was intended to prevent T cell activation in tissue transplant patients receiving a donor kidney, lung, or heart (Chatenoud and Bluestone, 2007). Many patients receiving muromonab-CD3 had adverse events including the induction of pro-inflammatory cytokines (for example cytokine storm), which was attributed in part to muromonab-CD3's interactions with FcγR's (Alegre et al., 1992). To reduce this unintended effector function, a human IgG1 variant L234A/L235A has been generated (Xu et al., 2000), which reduced inflammatory cytokine release. Reduced affinity of antibodies to the FcγRII receptor in particular would be advantageous for antibodies inducing platelet activation and aggregation via FcγRII receptor binding, which would be a serious side-effect of such antibodies.

Silenced effector functions can be obtained by Fc engineering. Various mutation sets are described in the art like LALA (L234A, L235A according to EU numbering) (Wines et al, 2000) or DAPA (D265A, P329A according to EU numbering) (Genentech, U.S. Pat. No. 6,737,056) for instance. Several investigators have employed a cross-subclass approach to reduce effector functions. In a further refinement of the cross-subclass approach, IgG2 variant was generated with point mutations from IgG4 (i.e., H268Q, V309L, A330S, P331S according to EU numbering) (An et al., 2009). Another silent IgG1 antibody comprises the N297A mutation, which results in aglycosylated/non-glycosylated antibodies (Strohl et al, 2009). Some used mutation sets combine previously described technologies, achieving higher levels of silencing up to completely abolishing some or all effector functions. DANAPA is one example (D265A, N297A, P329A) (WO2019068632 Janssen). Other alternate approaches to engineer or mutate critical residues in the Fc region that are responsible for effector functions have been reported. For examples see PCT publications WO 2009/100309 (Medimmune), WO 2006/076594 (Xencor), US 2006/0134709 (Macrogenics), U.S. Pat. No. 6,737,056 (Genentech), US 2010/0166740 (Roche).

Undesirable Fc interactions with Fcγ receptors and the complement receptor C1q can be decoupled from binding to the Neonatal Fc Receptor (FcRn) which can increase serum persistence. In vivo serum persistence conferred by FcRn is shown to be a tunable property that can be modulated by mutations in the IgG Fc. Increase the Fc affinity to FcRn in endosomal condition (acidic pH) by Fc engineering is an effective approach to prolong the pharmacokinetics of monoclonal antibodies (Maeda, 2017). YTE mutation set (M252Y, S254T, T256E according to EU numbering) or LS mutation set (M428L, N434S according to EU numbering) are examples of such developed mutation sets in Fc CH2 domains.

Chain pairing mutations have been demonstrated to be efficient at driving heavy-chain heterodimerization by introducing complementarity at the CH3-CH3 interface of bi-specific or multispecific antibodies. A number of chain pairing mutation sets are used in the production of multispecific antibodies: increasing/decreasing side-chain volume (T366W/S354C-T366S/L368A/Y407V/Y349C, knob-into-hole) (Ridgway, 1996), charge inversions (K409D/K392D-D399K/E356K, electrostatic steering) (Gunasekaran, 2010), or multiple IgA substitutions (SEEDbody) (Davis, 2010). However, all of these approaches make fairly substantial changes to the interface which result in destabilization and lower melting temperatures of the CH2 and CH3 regions (Kuglstatter, 2017; Garber, 2007).

Silenced effector functions, extended half-life (enhanced FcRn binding) or Fc chain pairing facilitating mutations achieved via Fc engineering represents great opportunity to improve and potentiate current immunotherapy. However, Fc modifications are known to alter physicochemical properties of the engineered antibodies. Modified therapeutic antibody can suffer from loss of thermostability, drop of expression yield, increase of aggregation propensity, decrease of solubility (Liu et al, 2013), leading to undesired outcomes for further therapeutics development (Yang et al, 2018). Moreover, many engineered Fc variants have potential immunogenicity problems, especially when extensive mutagenesis is involved to reduce the effector functions, as multiple mutation sites are likely to result in the formation of new epitopes.

Therefore, there remains a need for an effective method to compensate the destabilizing effects of the aforementioned mutation sets, comprising Fc silencing, half-life extending and/or chain pairing facilitating mutations, which would allow designing therapeutic antibodies with improved clinical, pharmaco-kinetic and-dynamic properties, extended half-life, improved manufacturing and formulating behavior and having improved Fc effector functions while retaining IgG like biophysical properties.

### SUMMARY

The present invention describes engineered immunoglobulin IgG Fc regions by transferring structural elements, i.e., several CH2 inter-chain disulfide bonds, from IgA to IgG immunoglobulin. The Fc variants created thereof exhibit marked reductions or complete abrogation of interaction of the engineered Fc with FcγR and C1q while retaining natural ability to interact with FcRn at acidic pH. The inventors discovered that eliminated antibody effector function could be achieved by a single cysteine substitution or in combinations of these, preferably single positions, selected from positions 234, 235 or 236. Resulting Fc molecules were in comparable expression and purification yields and improved or maintained thermostability compared to wild-type Fc, thereby limiting the propensity for aggregation. These substitutions are capable of reducing the destabilizing effects of YTE on the thermostability of engineered antibodies as well as KiH (knob-into-hole) chain pairing facilitating mutations. Furthermore, single cysteine substitutions, mimicking natural IgA, are anticipated to be less likely to generate any new epitopes, reducing risks of immunogenicity. Hence the current invention provides improved Fc modifications which can achieve greatly reduced to eliminated Fc effector functions but still retain stable desirable physicochemical properties similar to unmodified Fc with respect to yield, stability, melting temperature, solubility, aggregation propensity and other behavior in pharmaceutical formulations.

In one embodiment, provided herein is an engineered immunoglobulin (e.g. engineered antibodies) or fragment thereof comprising a Fc variant of a wild-type human IgG Fc polypeptide and one or more antigen binding domains, wherein the Fc variant exhibits reduced effector functions as compared to the wild-type human IgG Fc polypeptide, and wherein the Fc variant comprises one or more cysteine substitutions selected from the group consisting of positions: 234, 235, 236, 297 and 299, and wherein the amino acid residues are numbered according to the EU numbering. Cysteine 235 as found in IgA may substitute Leucine 235 in IgG CH2, but alternatively can also be positioned in the preceding Leucine at position 234 in IgG, as determined by studying the 3D crystal structures of these molecules. In fact, since concerned IgG1 amino acid are not exactly located at the same spatial position of equivalent IgA residue, some of the contiguous residues were also considered, such as L234 in particular, to form a stable sulfur bridge between both CH2 domains of the paired Fc molecules.

In a further embodiment, the one or more cysteine substitutions of the engineered immunoglobulin (e.g. engineered antibodies) or fragment thereof are selected from positions 234, 235 and 236. In one embodiment, the engineered immunoglobulin (e.g. engineered antibodies) or fragment thereof comprises a cysteine substitution at position 234. In another embodiment, the engineered immunoglobulin (e.g. engineered antibodies) or fragment thereof comprises a cysteine substitution at position 235. In another embodiment, the engineered immunoglobulin (e.g. engineered antibodies) or fragment thereof comprises a cysteine substitution at position 236.

In one embodiment, the engineered immunoglobulin (e.g. engineered antibodies) or fragment thereof further comprises: one or more amino acid substitutions in the Fc variant which enhance the half-life of the engineered immunoglobulin (e.g. engineered antibodies) or fragment thereof via enhanced FcRn binding and/or one or more amino acid substitutions that facilitate correct chain pairing of two different Fc chains.

In one embodiment, the half-life extending/FcRn binding enhancing amino acid substitutions are selected from the group consisting of mutation sets: M252Y/S254T/T256E (YTE), M428L/N434S (LS), T250Q/M428L (QL) and T307Q/N434A (QA).

In a further embodiment, the half-life extending/FcRn binding enhancing amino acid substitution is M252Y/S254T/T256E (YTE). In one embodiment, the engineered immunoglobulin (e.g. engineered antibodies) or fragment thereof comprises L234C and M252Y/S254T/T256E (YTE). In one embodiment, the engineered immunoglobulin (e.g. engineered antibodies) or fragment thereof comprises L235C and M252Y/S254T/T256E (YTE). In another embodiment, the engineered immunoglobulin (e.g. engineered antibodies) or fragment thereof comprises G236C and M252Y/S254T/T256E (YTE).

In another preferred embodiment, the half-life extending/FcRn binding enhancing amino acid substitution is M428L/N434S (LS). In one embodiment, the engineered immunoglobulin (e.g. engineered antibodies) or fragment thereof comprises L234C and M428L/N434S (LS). In one embodiment, the engineered immunoglobulin (e.g. engineered antibodies) or fragment thereof comprises L235C and M428L/N434S (LS). In another embodiment, the engineered immunoglobulin (e.g. engineered antibodies) or fragment thereof comprises G236C and M428L/N434S (LS).

In some embodiments, the engineered immunoglobulin (e.g. engineered antibodies) or fragment thereof is a human IgG1, IgG2, IgG3 or IgG4 antibody. Preferably, the engineered immunoglobulin (e.g. engineered antibodies) or fragment thereof is a human IgG1 antibody. In another preferred embodiment, the engineered immunoglobulin (e.g. engineered antibodies) or fragment thereof is a human IgG4 antibody.

In some embodiments, the engineered immunoglobulin (e.g. engineered antibodies) or fragment thereof is part of a multi-specific binding molecule (e.g. bispecific or trispecific or more specificities comprising antibody), which comprises chain pairing amino acid substitutions selected from the group consisting of knob-into-hole (Ridgway, 1996), SEEDbody (Davis, 2010), RF-mutation in half-Fc (Eliasson, 1988; Tustian, 2016), DEKK-mutation (De, 2017), electrostatic steering mutations (Gunasekaran, 2010), and Fab-arm exchange (Labrijn, 2011).

In one embodiment, the chain pairing amino acid substitutions are knob-into-hole (KiH) mutations, wherein the multispecific binding molecule comprises a first constant heavy chain with amino acid substitution of T366W and a second constant heavy chain with amino acid substitution of Y407T, and the amino acid residues are numbered according to the EU numbering.

In another embodiment, the chain pairing amino acid substitutions are knob-into-hole (KiH) mutations, wherein the multispecific binding molecule comprises a first constant heavy chain with amino acid substitution of T366W and a second constant heavy chain with amino acid substitutions of T366S, L368A and Y407V.

In a further embodiment, the chain paring amino acid substitutions are knob-into-hole (KiH) mutations, wherein the multispecific binding molecule comprises a first constant heavy chain with amino acid substitutions of S354C and T366W and a second constant heavy chain with amino acid substitutions of Y349C, T366S, L368A and Y407V, and the amino acid residues are numbered according to the EU numbering.

In one embodiment, the multispecific binding molecule comprises L234C and T366W/S354C-T366S/L368A/Y407V/Y349C (KiH). In another embodiment, the multispecific binding molecule comprises L235C and T366W/S354C-T366S/L368A/Y407V/Y349C (KiH). In another embodiment, the multispecific binding molecule comprises G236C, and T366W/S354C-T366S/L368A/Y407V/Y349C (KiH).

In one embodiment, the multispecific binding molecule comprises both T366W/S354C-T366S/L368A/Y407V/Y349C (KiH) and M252Y/S254T/T256E (YTE).

In one embodiment, the multispecific binding molecule comprises L234C, M252Y/S254T/T256E (YTE) and T366W/S354C-T366S/L368A/Y407V/Y349C (KiH). In one embodiment, the multispecific binding molecule comprises L235C, M252Y/S254T/T256E (YTE) and T366W/S354C-T366S/L368A/Y407V/Y349C (KiH). In another embodiment, the multispecific binding molecule comprises G236C, M252Y/S254T/T256E (YTE) and T366W/S354C-T366S/L368A/Y407V/Y349C (KiH).

Also provided herein is an engineered immunoglobulin or fragment thereof of to present disclosure for use as a medicament.

Also provided herein is a pharmaceutical composition comprising the engineered immunoglobulin (e.g. engineered antibodies) or fragment thereof of the present disclosure, in combination with one or more pharmaceutically acceptable excipient, diluent or carrier.

In one embodiment, the pharmaceutical composition further comprises one or more additional active agents.

Also provided herein is an isolated nucleic acid molecule, which encodes the engineered immunoglobulin (e.g. engineered antibodies) or fragment thereof of the present disclosure.

Also provided herein is a cloning or expression vector, which comprises one or more nucleic acid sequences as outlined above, wherein the vector is suitable for the recombinant production of the engineered immunoglobulin (e.g. engineered antibodies) or fragment thereof of the present disclosure.

Also provided herein is a host cell comprising one or more cloning or expression vectors as outlined above.

Also provided herein is a method for preparing the engineered immunoglobulin (e.g. engineered antibodies) or fragment thereof of present disclosure, the method comprising culturing a host cell as outlined above, purifying and recovering the engineered immunoglobulin (e.g. engineered antibodies) or fragment thereof from the host cell culture, and formulating the engineered immunoglobulin (e.g. engineered antibodies) or fragment thereof in a pharmaceutically acceptable composition.

### BRIEF DESCRIPTION OF THE FIGURES

FIG. 1 is a schematic overview of the tri-dimensional structures of both IgA2 and IgG1. FIG. 1A shows how both CH2 domains of IgA2 homodimer Fc are in contact to each other thanks to disulfide bonds and packed loops spatially located on top of IgA2 Fc (PDB 1OWO). FIG. 1B shows how both CH2 domains of IgG1 homodimer Fc stay distant from each other (PDB 1FC1). Finally, FIG. 1C describes IgG1 Fc and IgA2 Fc (respectively PDB 1FC1 and 1OWO) superimposed in 3D space, exhibiting main differences in the region on top of CH2 domain.

FIG. 2 shows a number of SDS-PAGE protein gels of engineered immunoglobulins expressed in HEK or CHO cells and purified by a 2-step purification process. FIG. 2A1 and FIG. 2A2 show SDS-PAGE analyses of engineered anti-CD3 monospecific IgG1 in non-reducing conditions. FIG. 2B shows SDS-PAGE analyses of engineered anti-CD3xTargetAxTargetB trispecific IgG1 in non-reducing conditions. FIG. 2C shows SDS-PAGE analyses of engineered IgG1 FC in non-reducing conditions. FIG. 2D shows SDS-PAGE analyses of engineered anti-CD3 monospecific IgG4 in non-reducing conditions. FIG. 2E shows SDS-PAGE analyses of engineered anti-TargetC monospecific IgG1 in non-reducing conditions.

FIG. 3 shows overall thermal stability of the engineered immunoglobulins compare to parental one. Data demonstrate that immunoglobulin engineering results in a more stable molecule with improved thermal stability over parental immunoglobulins. FIG. 3A shows overall thermal stability measurement done on engineered anti-CD3 monospecific hIgG1. FIG. 3B shows overall thermal stability measurement done on engineered CD3xTargetAxTargetB trispecific hIgG1. FIG. 3C shows overall thermal stability measurement done on engineered anti-CD3 monospecific hIgG4. FIG. 3D describes overall thermal stability measurement done on engineered anti-TargetC monospecific hIgG1.

FIG. 4 shows the thermal stability of engineered recombinant Fcs where measurement was done independently of the Fab.

FIG. 5 shows the NFAT activity of engineered anti-CD3 monospecific hIgG1, anti-CD3 monospecific hIgG4 and trispecific anti-CD3xTargetAxTargetB immunoglobulin. FIG. 5A presents results obtained using engineered anti-CD3 monospecific hIgG1 in separated assays (first assay: FIG. 5A1, second assay: FIG. 5A2, third assay: FIG. 5A3). In summary, parental (CD3_WT) and corresponding half-life extended variant (CD3_WT_YTE) show the greatest NFAT activity while all engineered immunoglobulins showed significantly dampened NFAT activation. FIG. 5B presents results obtained using engineered anti-CD3xTargetAxTargetB trispecific hIgG1. In summary, parental (CD3xTargetAxTargetB_WT) shows the greatest NFAT activity while all engineered immunoglobulins showed significantly dampened NFAT activation. FIG. 5C presents results obtained using engineered anti-CD3 monospecific hIgG4. In summary, parental (IgG4_CD3_WT or IgG4_CD3_S228P) and corresponding half-life extended variant (IgG4_CD3_WT_YTE or IgG4_CD3_S228P_YTE) show the greatest NFAT activity while all engineered immunoglobulins showed significantly dampened NFAT activation.

### DETAILED DESCRIPTION

Disclosed herein are engineered immunoglobulins, (e.g., engineered antibodies) or fragments thereof comprising mutated Fc regions such that the engineered immunoglobulins (e.g., engineered antibodies) can achieve eliminated effector functions but still retain stable the physicochemical properties.

### Definitions

In order that the present invention may be more readily understood, certain terms are defined throughout the detailed description. Unless defined otherwise, all technical and scientific terms used herein have the same meaning as commonly understood by those of ordinary skill in the art to which this invention pertains.

Unless stated otherwise, the following terms and phrases as used herein are intended to have the following meanings:

The terms “comprising” and “including” are used herein in their open-ended and non-limiting sense unless otherwise noted.

The term “binding molecule” of the present disclosure encompasses Fc containing binding molecules, full IgG, incl. IgG1, IgG4 antibodies, antibody variants, fragments of antibodies, antigen binding portions of antibodies that can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136) or can be multi-specific antibodies comprising an Fc domain and two or more binding moieties. In one embodiment, the Fc containing binding molecule of the present disclosure also comprises binding moieties such as nanobodies, Fabs, scFv's, Vhh's, DARPins, avimers, affibodies, Sso7d and anticalins.

As used herein, the term “antibody” refers to a polypeptide of the immunoglobulin family that is capable of binding a corresponding antigen non-covalently, reversibly, and in a specific manner. The basic functional unit of each antibody is an immunoglobulin monomer containing only one Ig unit, defined herein as an “Ig monomer”. Secreted antibodies can also be dimeric with two Ig units (e.g., IgA), tetrameric with four Ig units or pentameric with five Ig units (e.g., mammalian IgM). The Ig monomer is a Y-shaped molecule that consists of four polypeptide chains; two identical heavy chains and two identical light chains connected by disulfide bonds (Woof & Burton (2004) Nature Reviews Immunology, 4(2): 89-99). Each chain comprises a number of structural domains containing about 70-110 amino acids that are classified into two categories: variable or constant, according to their size and function. The heavy chain comprises one variable domain (variable heavy chain domain; abbreviated as VH) and three constant domains (abbreviated as CH1, CH2 and CH3). Each light chain comprises one variable domain (abbreviated as VL) and one constant domain (abbreviated as CL). Immunoglobulin domains have a characteristic immunoglobulin fold in which two beta sheets create a ‘sandwich’ shape, held together by interactions between conserved cysteine residues and other charged amino acids. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain an antigen binding domain or antigen binding site that interacts with an antigen.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

The terms “recognize” or “bind” as used herein refers to a binding molecule, an antibody or antigen-binding fragment thereof that finds and interacts (e.g., binds or recognizes) its epitope, whether that epitope is linear, discontinuous or conformational. The term “epitope” refers to a site on an antigen to which an antibody or antigen-binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one aspect, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some aspects, antibodies or antibody fragments are selected that cross-react with certain desired molecules.

The term “antigen-binding site” refers to the part of an antibody that comprises determinants that form an interface that binds to the antigen, or an epitope thereof. The term “antigen binding site” may be used interchangeably with the term “antigen binding domain” or antigen binding moiety. With respect to proteins (or protein mimetics), the antigen-binding site typically includes one or more loops (of at least four amino acids or amino acid mimics) that form an interface that binds to the antigen polypeptide. Typically, the antigen-binding site of an antibody molecule includes at least one or two CDRs and/or hypervariable loops, or more typically at least three, four, five or six CDRs and/or hypervariable loops.

“Complementarity-determining regions” (“CDRs”) as used herein, refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting in total about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby (2000) Immunology, 4th ed., Chapter 4. W.H. Freeman & Co., New York). The term “monoclonal antibody” or “monoclonal antibody composition” as used herein refers to polypeptides, including antibodies and antigen-binding fragments that have substantially identical amino acid sequence or are derived from the same genetic source. This term also includes preparations of antibody molecules of single molecular composition. A monoclonal antibody composition displays a single binding specificity and affinity for a particular epitope. Methods for generation of monoclonal antibodies using phage display technology are known in the art (Proetzel, G., Ebersbach, H. (Eds.) Antibody Methods and Protocols. Humana Press ISBN 978-1-61779-930-3; 2012).

The term “human antibody,” as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000). In a preferred embodiment, the engineered IgG immunoglobulin or fragment thereof of the present disclosure is a human antibody.

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

An antibody or immunoglobulin can be one in which the variable region, or a portion thereof, e.g., the CDRs, are generated in a non-human organism, e.g., a rat or mouse. Chimeric, CDR-grafted, and humanized antibodies are within the invention. An antibody can be humanized by methods known in the art (see e.g., Morrison, S. L., (1985), Science 229:1202-1207; Oi et al., (1986), BioTechniques 4:214, and Queen et al., U.S. Pat. Nos. 5,585,089, 5,693,761 and 5,693,762, the contents of all of which are hereby incorporated by reference). Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al., (1986) Nature 321:552-525; Verhoeyan et al., (1988) Science 239:1534; Beidler et al., (1988) J. Immunol. 141:4053-4060 and Winter U.S. Pat. No. 5,225,539, the contents of all of which are hereby expressly incorporated by reference. Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, the contents of which are hereby incorporated by reference. Other techniques for humanizing antibodies are described in Padlan et al., EP 519596 A1.

In mammals there are two types of immunoglobulin light chain, which are called lambda (λ) and kappa (κ). Each antibody contains two light chains that are always identical; only one type of light chain, κ or λ, is present per antibody in mammals. The approximate length of a light chain is 211 to 217 amino acids and each light chain has two domains, one constant domain and one variable domain.

There are five types of mammalian Ig heavy chains denoted α, δ, ε, γ, and μ and the type of heavy chain present in the antibody defines the class or isotype of the antibody: IgM, IgG, IgA, IgD, IgE, respectively. The heavy chains vary in physiochemical, structural, and immunological properties but each heavy chain has two domains, a variable domain and a constant domain. The variable domain comprises a single Ig domain (approximately 110 amino acids long) and determines antibody binding specificity. The constant domain is identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains (Woof & Burton, supra). In one embodiment, an “immunoglobulin” can be an antibody. In an embodiment, a “fragment thereof” of an immunoglobulin can be an Fc region or one or more Fc domains.

The term “Fc region” refers to the fragment crystallisable region of an antibody, which plays an important role in modulating immune cell activity. The Fc Region is composed of two polypeptide chains or Fc domains, which in IgG comprises the CH2 and CH3 constant domains or ‘CH2 domain’ and ‘CH3 domain’ respectively, of the heavy chain. IgM and IgE Fc regions contain three heavy chain constant domains (CH domains 2-4) in each polypeptide chain. The amino acid residues in the CH2 and CH3 domains can be numbered according to the EU numbering system (Edelman et al., (1969) PNAS. USA, 63, 78-85), “Kabat” numbering (Kabat et al., supra) or alternatively using the IMGT numbering for C domains. IMGT tools are available at world wide web (www.imgt.org).

The Fc region binds to cell surface receptors, “Fc receptors” and complement proteins mediating physiological effects of antibodies. Fc receptors are found on may cells of the immune system including: B lymphocytes, follicular dendritic cells, natural killer cells, macrophages, neutrophils, eosinophils, basophils, human platelets and mast cells. Binding of antibody Fc region to Fc receptors stimulates phagocytic or cytotoxic cells to destroy microbes, or infected cells by the mechanism of antibody-dependent cell-mediated cytotoxicity (ADCC). There are several different types of Fc receptors (FcR), which are classified based on the type of antibody that they recognize. For example, those that bind IgG are called Fc-gamma receptors (FcγR), those that bind IgA are called Fc-alpha receptors (FcαR) and those that bind IgE are called Fc-epsilon receptors (FcεR). The classes of FcRs are also distinguished by the cells that express them (macrophages, granulocytes, natural killer cells, T and B cells) and the signaling properties of each receptor (Owen J et al., (2009) Immunology (7th ed.). New York: W.H. Freeman and Company. p 423). The following table (Table 1) summarizes the different Fc receptors, their ligands, cell distribution and binding effects.

TABLE 1
Summary of Fc receptors and their properties

| | Principal | | | |
|---|---|---|---|---|
| Receptor | antibody | | | |
| name | ligand | Affinity for ligand | Cell distribution | Effect following binding to antibody |
| FcγRI | IgG1 and | High (Kd ~10⁻⁹M) | Macrophages | Phagocytosis |
| (CD64) | IgG3 | | Neutrophils | Cell activation |
| | | | Eosinophils | Activation of respiratory burst |
| | | | Dendritic cells | Induction of microbe killing |
| FcγRIIA | IgG | Low (Kd > 10⁻⁷M) | Macrophages | Phagocytosis |
| (CD32) | | | Neutrophils | Degranulation (eosinophils) |
| | | | Eosinophils | |
| | | | Platelets | |
| | | | Langerhans cells | |
| FcγRIIB1 | IgG | Low (Kd > 10⁻⁷M) | B Cells | No phagocytosis |
| (CD32) | | | Mast cells | Inhibition of cell activity |
| FcγRIIB2 | IgG | Low (Kd > 10⁻⁷M) | Macrophages | Phagocytosis |
| (CD32) | | | Neutrophils | Inhibition of cell activity |
| | | | Eosinophils | |
| FcγRIIIA | IgG | Low (Kd > 10⁻⁶M) | NK cells | Induction of antibody-dependent cell- |
| (CD16a) | | | Macrophages | mediated cytotoxicity (ADCC) |
| | | | (certain tissues) | Induction of cytokine release by |
| | | | | macrophages |
| FcγRIIIB | IgG | Low (Kd > 10⁻⁶M) | Eosinophils | Induction of microbe killing |
| (CD16b) | | | Macrophages | |
| | | | Neutrophils | |
| | | | Mast cells | |
| | | | Follicular dendritic | |
| | | | cells | |
| FcεRI | IgE | High (Kd ~10⁻¹⁰M) | Mast cells | Degranulation |
| | | | Eosinophils | Phagocytosis |
| | | | Basophils | |
| | | | Langerhans cells | |
| | | | Monocytes | |
| FcεRII | IgE | Low (Kd > 10⁻⁷M) | B cells | Possible adhesion molecule |
| (CD23) | | | Eosinophils | IgE transport across human intestinal |
| | | | Langerhans cells | epithelium |
| | | | | Positive-feedback mechanism to |
| | | | | enhance allergic sensitization (B cells) |
| FcαRI | IgA | Low (Kd > 10⁻⁶M) | Monocytes | Phagocytosis |
| (CD89) | | | Macrophages | Induction of microbe killing |
| | | | Neutrophils | |
| | | | Eosinophils | |
| Fcα/μR | IgA and | High for IgM, | B cells | Endocytosis |
| | IgM | Mid for IgA | Mesangial cells | Induction of microbe killing |
| | | | Macrophages | |
| FcRn | IgG | Low (Kd > 10⁻⁶M) | Monocytes | Transfers IgG from a mother to fetus |
| | | | Macrophages | through the placenta |
| | | | Dendritic cells | Transfers IgG from a mother to infant |
| | | | Epithelial cells | in milk |
| | | | Endothelial cells | Protects IgG from degradation |
| | | | Hepatocytes | |

A “modification” or “mutation” of an amino acid residue(s)/position(s), as used herein, refers to a change of a primary amino acid sequence as compared to a starting amino acid sequence, wherein the change results from a sequence alteration involving said one or more amino acid residue/positions. For example, typical modifications include substitution of the one or more residue(s) (or at said position(s)) with another amino acid(s) (e.g., a conservative or non-conservative substitution), insertion of one or more amino acids adjacent to said one or more residue(s)/position(s), and deletion of said one or more residue(s)/position(s), inversion of said one or more residue(s)/position(s), and duplication of said one or more residue(s)/position(s).

An “amino acid substitution” or “substitution”, refers to the replacement of one or more existing amino acid residue(s) in a predetermined (starting or parent) amino acid sequence with a one or more different amino acid residue(s). For example, the substitution 1332E refers to a variant polypeptide, in this case a constant heavy chain variant, in which the isoleucine at position 332 is replaced with glutamic acid (EU numbering). Alternatively, the position of the substitution in the CH2 or CH3 domain can be given, for example, CH2.97 indicates a substitution at position 97 in a CH2 domain with the numbering according to IMGT numbering for C-domain. The exact substitution can also be indicated by, for example, L_CH2.97_Y, which indicates that the leucine at position 97 in a CH2 domain is replaced by tyrosine.

Generally and preferably, the modification results in alteration in at least one physicobiochemical activity of the variant polypeptide compared to a polypeptide comprising the starting (or “wild-type”) amino acid sequence. For example, in the case of an antibody or a multispecific binding molecule, a physicobiochemical activity that is altered can be binding affinity, binding capability and/or binding effect upon a target molecule.

The term “in vivo half-life”, as used herein, refers to the half-life of the molecule of interest or variants thereof circulating in the blood of a given mammal.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine (K), arginine (R), histidine (H)), acidic side chains (e.g., aspartic acid (D), glutamic acid (E)), uncharged polar side chains (e.g., glycine (G), asparagine (N), glutamine (Q), serine(S), threonine (T), tyrosine (Y), cysteine (C)), nonpolar side chains (e.g., alanine (A), valine (V), leucine (L), isoleucine (I), proline (P), phenylalanine (F), methionine (M), tryptophan (W)), beta-branched side chains (e.g., threonine (T), valine (V), isoleucine (I)) and aromatic side chains (e.g., tyrosine (Y), phenylalanine (F), tryptophan (W), histidine (H)).

The terms “percent identical” or “percent identity” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. A “percentage identity” or “percentage sequence identity” of the present disclosure can be calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

Other than percentage of sequence identity noted above, another indication that two nucleic acid sequences or polypeptides are substantially identical is that the polypeptide encoded by the first nucleic acid is immunologically cross reactive with antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Various aspects of the disclosure are described in further detail in the following sections and subsections.

### Detailed Description of Generation of Engineered Immunoglobulins (e.g., Engineered Antibodies) With Highly Diminished or Eliminated Fc Effector Functions

To generate IgG immunoglobulins with these stabilizing and silencing properties in the Fc portion, crystal structure analysis of IgG1 and IgA1 Fc regions was compared to identify the specific amino acid residues in an IgA immunoglobulin that are critical for binding to FcγR. The basic monomer unit of IgG or IgA, in common with all antibodies, is arranged into two identical Fab regions, linked through the hinge region to the Fc. Both heavy and light chains are folded into globular domains, four in each heavy chain (from the N-terminus VH, CH1, CH2, and CH3) and two in each light chain (VL and CL). Each IgG and IgA domain adopts the characteristic “immunoglobulin fold”, comprising a 110 residue β-sheet sandwich of anti-parallel strands arranged around a stabilizing internal disulfide bond. There is close pairing of domains between neighboring chains (VH with VL, CH1 with CL, and CH3 with CH3) and inter-chain disulfide bridges further stabilize the structure. In IgG immunoglobulin, these are found between heavy chain in the hinge region (C226 and C229 according to EU numbering) contrary to IgA where these inter-chain disulfide bounds are found between heavy chains in the CH2 domain. Available X-ray crystal structures (Herr et al, 2003; Ramsland et al, 2007) implicate four potential cysteines on each heavy chain (C235, C236, C297, C299 according to EU numbering) in linkages across the upper parts of the CH2 domains. The precise arrangements differ in the solved structures for IgA1 Fc complexes with different ligands, suggesting that a degree of disulfide interchange may be possible (Woof et al, 2011).

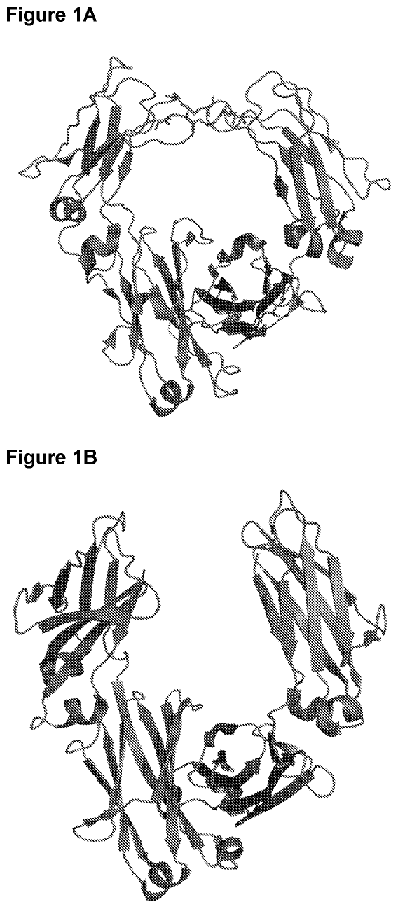

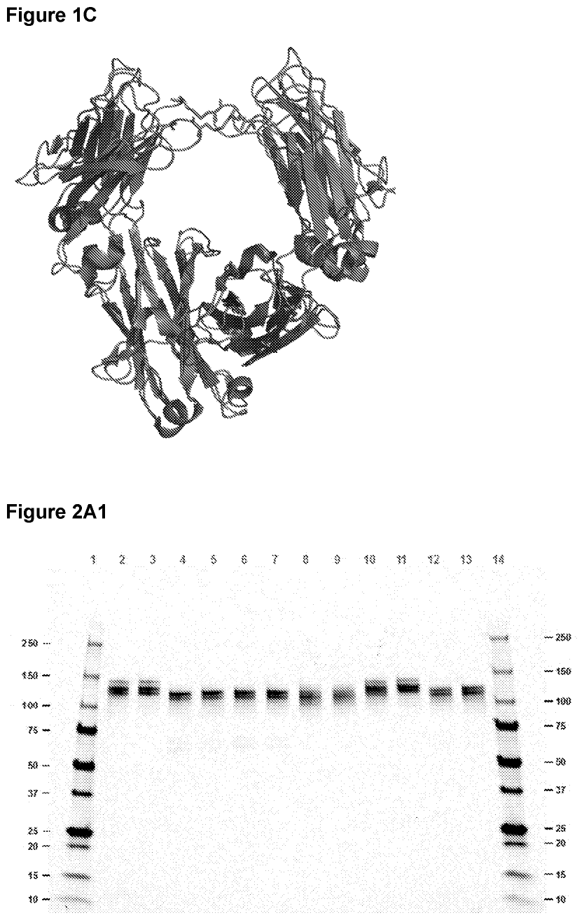

Regarding IgA, both CH2 domains of the homodimer Fc are coming into contact and are linked together by four disulfide bonds at positions C235, C236, C297 and C299 (according to EU numbering). This packed region observed on top of IgA CH2 region is described FIG. 1A. Contrary to IgA, IgG does not have this disulfide bridging. In fact, both CH2 domains of IgG homodimer Fc stay distant from each other, as described FIG. 1B. As a result, CH2 domains of both IgG and IgA homodimer Fc do not share the same 3D position and orientation within the Fc. Those observations are shown FIG. 1C by 3D superimposition of both IgG and IgA Fc's.

In present invention, such IgA structural element was introduced into IgG Fc by substitution of concerned positions by IgA amino acids, involved in this top CH2 packed region. Since concerned IgG1 amino acid are not exactly located at the same spatial position of equivalent IgA residue, some of the contiguous residues were also considered, such as L234 in particular.

Mutation sets introduced into IgG FC are described Table 2.

TABLE 2
Tested mutation sets, used to transfer top IgA CH2 structural element into IgG1 FC.

| Mutation set ID | Mutations (according to EU numbering) |
|---|---|
| 1 | L235C, G236C, G237H, S239R, V264T, D265G, V266L, S267R, |
| | H268D, N297C, S298G, T299C, R301S |
| 2 | L235C, G236C, G237H, D265G, V266L, S267R, N297C, S298G, T299C |
| 3 | L235C, G236C, G237H, V264T, D265G, V266L, S267R, H268D, |
| | N297C, S298G, T299C |
| 4 | L235C, G236C, G237H, N297C, S298G, T299C |
| 5 | L235C, G236C, N297C, T299C |
| 6 | L235C, G236C |
| 7 | N297C, T299C |
| 8 | G236C |
| 9 | L235C |
| 10 | T299C |
| 11 | N297C |
| 12 | L235C, N297C |
| 13 | L234C, L235C |
| 14 | L234C |

As demonstrated in present disclosure, transfer of CH2 inter-chain disulfide bonds from IgA to IgG immunoglobulin enabled the generation of an engineered immunoglobulin (e.g. an engineered antibody) or fragment thereof comprising an IgG Fc variant with eliminated Fc effector functions. In one embodiment, the present disclosure provides an engineered IgG immunoglobulin comprising one or more cysteine substitutions selected from the group consisting of positions: 234, 235, 236, 297 and 299, and wherein the amino acid residues are numbered according to the EU numbering.

In one embodiment, the present disclosure provides an engineered IgG immunoglobulin (e.g. an engineered antibody) or fragment thereof comprising one or more cysteine substitutions selected from the group consisting of positions 234, 235 and 236 in the Fc domain.

In some embodiments, the engineered IgG immunoglobulin is a human IgG1, IgG2, IgG3 or IgG4. In one embodiment, the Fc variant has an amino acid sequence identity to an Fc domain from wild-type human IgG1 of at least 90%. In one embodiment, the Fc variant has an amino acid sequence identity to an Fc domain from wild-type human IgG1 of at least 95%. In one embodiment, the Fc variant has an amino acid sequence identity to an Fc domain from wild-type human IgG1 of at least 98%. In one embodiment, the Fc variant has an amino acid sequence identity to an Fc domain from wild-type human IgG4 of at least 90%. In one embodiment, the Fc variant has an amino acid sequence identity to an Fc domain from wild-type human IgG4 of at least 95%. In one embodiment, the Fc variant has an amino acid sequence identity to an Fc domain from wild-type human IgG4 of at least 98%.

In one embodiment, the present disclosure provides an engineered IgG immunoglobulin (e.g. an engineered antibody) or fragment thereof further comprising any one of the described mutation-sets in Table 2 wherein the amino acid residues are numbered according to the EU numbering. In one embodiment, the engineered IgG immunoglobulin (e.g. an engineered antibody) or fragment thereof further comprises: one or more amino acid substitutions in the Fc variant which enhance the half-life of the engineered IgG immunoglobulin (e.g. an engineered antibody) or fragment thereof via enhanced FcRn binding and/or one or more amino acid substitutions that facilitate correct chain pairing of two different Fc chains. In a preferred embodiment, the half-life extending/FcRn binding enhancing amino acid substitutions are selected from the group consisting of mutation sets: M252Y/S254T/T256E (YTE), M428L/N434S (LS) and T250Q/M428L(QL) and T307Q/N434A(QA).

It is known that the YTE mutant has lower physical stability than the same mAb without the mutations (Tavakoli-Keshe, 2014). One possibility is that these stability differences are mediated by changes in structural dynamics of specific sequences in the mAbs due to the YTE mutations. Surprisingly, the present invention shows that the cysteine substitutions are capable of reducing the destabilization effect of YTE mutations on the engineered IgG immunoglobulin (e.g. an engineered antibody) or fragment thereof. In one embodiment, the engineered IgG immunoglobulin (e.g. an engineered antibody) or fragment thereof comprises L234C and M252Y/S254T/T256E (YTE). In one embodiment, the engineered IgG immunoglobulin (e.g. an engineered antibody) or fragment thereof comprises L235C and M252Y/S254T/T256E (YTE). In another embodiment, the engineered IgG immunoglobulin (e.g. an engineered antibody) or fragment thereof comprises G236C and M252Y/S254T/T256E (YTE).

The cysteine substitutions provided by present invention are also capable of reducing the destabilization effect of LS mutations. In a preferred embodiment, the engineered IgG immunoglobulin (e.g. an engineered antibody) or fragment thereof comprises L234C and M428L/N434S (LS). In one embodiment, the engineered IgG immunoglobulin (e.g. an engineered antibody) or fragment thereof comprises L235C and M428L/N434S (LS). In another embodiment, the engineered IgG immunoglobulin (e.g. an engineered antibody) or fragment thereof comprises G236C and M428L/N434S (LS).

In one embodiment, the engineered IgG immunoglobulin (e.g. an engineered antibody) or fragment thereof comprising any one of the described mutation sets in Table 2 is a monospecific antibody.

In one embodiment, the monospecific antibody comprises L234C and M252Y/S254T/T256E (YTE). In one embodiment, the monospecific antibody comprises L235C and M252Y/S254T/T256E (YTE). In another embodiment, the monospecific antibody comprises G236C and M252Y/S254T/T256E (YTE).

In one embodiment, the engineered IgG immunoglobulin (e.g. an engineered antibody) or fragment thereof comprising any one of the described mutation sets in Table 2 as described above is a multispecific antibody, in particular a bi- or tri-specific antibody.

The term “monospecific molecule,” as used herein, refers to an Fc containing molecule that binds to one epitope on a target antigen. In some embodiments, a mono-specific molecule of the present disclosure is a monospecific antibody-like molecule. In some embodiments, a mono-specific molecule of the present disclosure is a monospecific antibody. The term “bispecific molecule” refers to a multispecific Fc containing binding molecule that binds to two different antigens. The term “trispecific molecule” refers to an Fc containing multispecific binding molecule that binds to three different antigens via three different binding moieties. In some embodiments, a bispecific molecule of the present disclosure is a bispecific antibody-like molecule. In some embodiments, a multispecific binding molecule of the present disclosure is a multispecific antibody-like molecule.

The term “multispecific antibody” refers to antibody capable of recognizing two or more epitopes of an antigen or two or more antigens. Recognition of each antigen is generally accomplished via an “antigen-binding domain”. In particular, bispecific antibodies recognize two different epitopes either on the same or on different antigens. All bispecific IgG molecules, i.e., bispecific antibodies indistinguishable in their composition from natural immunoglobulins, are bivalent and possess an asymmetric architecture due to the presence of, at least, different Fv regions. Depending on the method of preparation and origin of heavy and light chains, they may furthermore differ in the constant regions of the heavy or light chain (Brinkmann and Kontermann, 2017).

In one embodiment, the bispecific antibody further comprises half-life extending mutations, e.g., M252Y/S254T/T256E (YTE). In one embodiment, the bispecific antibody comprises L234C and M252Y/S254T/T256E (YTE). In one embodiment, the bispecific antibody comprises L235C and M252Y/S254T/T256E (YTE). In another embodiment, the bispecific antibody comprises G236C and M252Y/S254T/T256E (YTE).

In a preferred embodiment, the multispecific antibody comprises mutations which promote correct HC/HC pairing.

To ensure adequate heterodimerization of the two Fc domains of the Fc region of the engineered immunoglobulin (e.g., an engineered antibody) or fragment thereof of the present disclosure, a variety of approaches can be used in to enhance dimerization, as described in e.g., EP1870459; U.S. Pat. Nos. 5,582,996; 5,731,168; 5,910,573; 5,932,448; 6,833,441; 7,183,076; US2006204493A1; WO 09/089004A1. In one embodiment, one or more mutations to a first Fc domain of the engineered immunoglobulin (e.g., an engineered antibody) or fragment thereof comprising a heavy chain constant domain creates a “knob” and the one or more mutations to a second Fc domain of the engineered immunoglobulin (e.g. an engineered antibody) or fragment thereof comprising a heavy chain constant domain creates a “hole,” such that heterodimerization of the first and second Fc domains causes the “knob” to interface (e.g., interact, e.g., a CH2 domain of a first Fc domain interacting with a CH2 domain of a second Fc domain, or a CH3 domain of a first Fc domain interacting with a CH3 domain of a second Fc domain) with the “hole”.

As the term is used herein, a “knob” refers to at least one amino acid side chain which projects from the interface of a first Fc domain of the engineered immunoglobulin (e.g. an engineered antibody) or fragment thereof comprising a heavy chain constant domain and is therefore positionable in a compensatory “hole” in the interface with a second Fc domain of the engineered immunoglobulin (e.g. an engineered antibody) or fragment thereof comprising a heavy chain constant domain so as to stabilize the heterodimer, and thereby favour heterodimeric formation over homodimeric formation, for example. The preferred import residues for the formation of a knob are generally naturally occurring amino acid residues and are preferably selected from arginine (R), phenylalanine (F), tyrosine (Y) and tryptophan (W). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second Fc domain of the engineered immunoglobulin (e.g. an engineered antibody) or fragment thereof comprising a heavy chain constant domain and therefore accommodates a corresponding knob on the adjacent interfacing surface of a first Fc domain of the engineered immunoglobulin (e.g. an engineered antibody) or fragment thereof comprising a heavy chain constant domain. The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In one embodiment, the chain pairing amino acid substitutions are knob-into-hole (KiH) mutations, wherein the multispecific binding molecule comprises a first constant heavy chain with amino acid substitution of T366W and a second constant heavy chain with amino acid substitution of Y407T, and the amino acid residues are numbered according to the EU numbering.

In another embodiment, the chain pairing amino acid substitutions are knob-into-hole (KiH) mutations, wherein the multispecific binding molecule comprises a first constant heavy chain with amino acid substitution of T366W and a second constant heavy chain with amino acid substitutions of T366S, L368A and Y407V, and the amino acid residues are numbered according to the EU numbering.

In a further embodiment, the chain paring amino acid substitutions are knob-into-hole (KiH) mutations, wherein the multispecific binding molecule comprises a first constant heavy chain with amino acid substitutions of S354C and T366W and a second constant heavy chain with amino acid substitutions of Y349C, T366S, L368A and Y407V, and the amino acid residues are numbered according to the EU numbering.

In one embodiment, the multispecific antibody comprises L234C and the KiH mutations as described above; comprising a first constant heavy chain with amino acid substitutions of S354C and T366W and a second constant heavy chain with amino acid substitutions of Y349C, T366S, L368A and Y407V. In one embodiment, the multispecific antibody comprises L235C and the KiH mutations in a first constant heavy chain with amino acid substitutions of S354C and T366W and a second constant heavy chain with amino acid substitutions of Y349C, T366S, L368A and Y407V. In another embodiment, the multispecific antibody comprises G236C and the KiH mutations in a first constant heavy chain with amino acid substitutions of S354C and T366W and a second constant heavy chain with amino acid substitutions of Y349C, T366S, L368A and Y407V.

In a yet another preferred embodiment, the multispecific antibody comprises L234C, KiH and YTE mutations. In one embodiment, the bispecific antibody comprises L235C, KiH and YTE mutations. In another embodiment, the bispecific antibody comprises G236C, KiH and YTE mutations.

In some cases, HC/LC pairing was driven by electro-steering, introducing following mutation sets on HC and LC:
Q38K, Q124D, K169 in Kappa LC,
Q39D, K147, S165D in HC
Q38D, E124K, N170D in Lambda LC
Q39K, K147D, S165R in HC

In a further embodiment, the multispecific antibody is produced by combining knob-into-hole strategy with electrostatic steering method.

In one embodiment, the engineered IgG immunoglobulin (e.g. an engineered antibody) or fragment thereof comprising any one of the described mutation sets in Table 2 is a bispecific antibody.

In one embodiment, the bispecific antibody further comprises half-life extending mutations, e.g., M252Y/S254T/T256E (YTE). In one embodiment, the bispecific antibody comprises L234C and M252Y/S254T/T256E (YTE). In one embodiment, the bispecific antibody comprises L235C and M252Y/S254T/T256E (YTE). In another embodiment, the bispecific antibody comprises G236C and M252Y/S254T/T256E (YTE).

In a preferred embodiment, the bispecific antibody comprises mutations which promotes correct HC/HC pairing, wherein the mutations which promotes correct HC/HC pairing may be knob-in-hole or the electrostatic steering method, or the combination of both.

In one embodiment, the bispecific antibody comprises L234C and the KiH mutations in a first constant heavy chain with amino acid substitutions of S354C and T366W and a second constant heavy chain with amino acid substitutions of Y349C, T366S, L368A and Y407V. In one embodiment, the bispecific antibody comprises L235C and the KiH mutations in a first constant heavy chain with amino acid substitutions of S354C and T366W and a second constant heavy chain with amino acid substitutions of Y349C, T366S, L368A and Y407V. In another embodiment, the bispecific antibody comprises G236C and the KiH mutations in a first constant heavy chain with amino acid substitutions of S354C and T366W and a second constant heavy chain with amino acid substitutions of Y349C, T366S, L368A and Y407V.

In a preferred embodiment, the bispecific antibody comprises L234C, KiH and YTE mutations. In one embodiment, the bispecific antibody comprises L235C, KiH and YTE mutations. In another embodiment, the bispecific antibody comprises G236C, KiH and YTE mutations.

In one embodiment, the engineered IgG immunoglobulin (e.g. an engineered antibody) or fragment thereof comprising any one of the described mutation sets in Table 2 is a trispecific antibody.

In one embodiment, the trispecific antibody further comprises half-life extending mutations, e.g., M252Y/S254T/T256E (YTE). In one embodiment, the trispecific antibody comprises L234C and M252Y/S254T/T256E (YTE). In one embodiment, the trispecific antibody comprises L235C and M252Y/S254T/T256E (YTE). In another embodiment, the trispecific antibody comprises G236C and M252Y/S254T/T256E (YTE).

In a preferred embodiment, the trispecific antibody comprises mutations which promotes correct HC/HC pairing, wherein the mutations which promotes correct HC/HC pairing may be knob-in-hole or the electrostatic steering method, or the combination of both.

In one embodiment, the trispecific antibody comprises L234C and the KiH mutations in a first constant heavy chain with amino acid substitutions of S354C and T366W and a second constant heavy chain with amino acid substitutions of Y349C, T366S, L368A and Y407V. In one embodiment, the trispecific antibody comprises L235C and the KiH mutations in a first constant heavy chain with amino acid substitutions of S354C and T366W and a second constant heavy chain with amino acid substitutions of Y349C, T366S, L368A and Y407V. In another embodiment, the trispecific antibody comprises G236C and the KiH mutations in a first constant heavy chain with amino acid substitutions of S354C and T366W and a second constant heavy chain with amino acid substitutions of Y349C, T366S, L368A and Y407V.

In a preferred embodiment, the trispecific antibody comprises L234C, KiH and YTE mutations. In one embodiment, the trispecific antibody comprises L235C, KiH and YTE mutations. In another embodiment, the trispecific antibody comprises G236C, KiH and YTE mutations.

Fc fragments of human IgG were also produced, comprising any one of the described mutation sets in Table 2.

In one embodiment, the Fc fragment further comprises half-life extending mutations, e.g., M252Y/S254T/T256E (YTE). In one embodiment, the Fc fragment comprises L234C and M252Y/S254T/T256E (YTE). In one embodiment, the Fc fragment comprises L235C and M252Y/S254T/T256E (YTE). In another embodiment, the Fc fragment comprises G236C and M252Y/S254T/T256E (YTE).

In a preferred embodiment, the Fc fragment comprises mutations which promotes correct HC/HC pairing, wherein the mutations which promotes correct HC/HC pairing may be knob-in-hole or the electrostatic steering method, or the combination of both.

In one embodiment, the Fc fragment comprises L234C and the KiH mutations as described above. In one embodiment, the Fc fragment comprises L235C and the KiH mutations. In another embodiment, the Fc fragment comprises G236C and the KiH mutations.

In a preferred embodiment, the Fc fragment comprises L234C, KiH and YTE mutations. In one embodiment, the Fc fragment comprises L235C, KiH and YTE mutations. In another embodiment, the Fc fragment comprises G236C, KiH and YTE mutations.

Also provided herein is an engineered immunoglobulin or fragment thereof of to present disclosure for use as a medicament.

Also provided herein is an engineered immunoglobulin or fragment thereof of to present disclosure for use in a therapy.

Protein and corresponding nucleotide sequences of the engineered immunoglobulins and Fc fragments are described Table 8.

### Nucleic Acids and Expression Systems

The present invention also encompasses isolated nucleic acids encoding the polypeptide chains of the engineered immunoglobulin (e.g., engineered antibodies) or fragment thereof of present disclosure. Nucleic acid molecules of the disclosure include DNA and RNA in both single-stranded and double-stranded form, as well as the corresponding complementary sequences. The nucleic acid molecules of the disclosure include full-length genes or cDNA molecules as well as a combination of fragments thereof. The nucleic acids of the disclosure are derived from human sources but can include those derived from non-human species.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, NY (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Variant sequences can be prepared by site specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein.

As “optimized nucleotide sequence” means a nucleotide sequence has been altered to encode an amino acid sequence using codons that are preferred in the production cell, for example, a Chinese Hamster Ovary cell (CHO). The optimized nucleotide sequence is engineered to retain completely the amino acid sequence originally encoded by the starting nucleotide sequence, which is also known as the “parental” sequence.

The present disclosure also provides expression systems and constructs in the form of plasmids, expression vectors, transcription or expression cassettes which comprise at least one polynucleotide as above. In addition, the disclosure provides host cells comprising such expression systems or constructs. The heavy and light chains of an engineered IgG immunoglobulin or fragment thereof can be encoded by a single nucleic acid (e.g., inserted into a single vector), or can be encoded by multiple nucleic acid molecules, e.g., two nucleic acid molecules (also referred to as a “set”), which can be inserted into multiple vectors (e.g., two vectors, i.e., a set of vectors).

In one embodiment, a method of preparing an engineered IgG immunoglobulin or fragment thereof comprising an Fc variant disclosed in the present invention is provided, the method comprising the steps of: (a) culturing a host cell comprising a nucleic acid encoding a heavy chain comprising the engineered Fc domain polypeptide and a nucleic acid comprising a light chain polypeptide, wherein the cultured host cell expresses the engineered polypeptides; and (b) purifying and recovering the engineered IgG immunoglobulin or fragment thereof from the host cell culture. Optionally, the method may comprise a further step (c) of formulating the IgG immunoglobulin or fragment thereof in a pharmaceutically acceptable composition.

A cloning or expression vector is provided, which comprises one or more nucleic acid sequences as described above, wherein the vector is suitable for the recombinant production of the engineered immunoglobulins (e.g., engineered antibodies) of present disclosure or fragment thereof.

Expression vectors of use in the present disclosure may be constructed from a starting vector such as a commercially available vector. After the vector has been constructed and a nucleic acid molecule encoding polypeptide chains of the engineered immunoglobulin has been inserted into the proper site of the vector, the completed vector may be inserted into a suitable host cell for amplification and/or polypeptide expression. The transformation of an expression vector into a selected host cell may be accomplished by known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

Typically, expression vectors used in the host cells will contain sequences for plasmid maintenance and for cloning and expression of exogenous nucleotide sequences. Such sequences, collectively referred to as ‘flanking sequences’, in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

A host cell is also provided, comprising one or more cloning or expression vectors of the present disclosure.

A host cell, when cultured under appropriate conditions, can be used to express the engineered immunoglobulins (e.g., engineered antibodies) or fragment thereof that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. A host cell may be eukaryotic or prokaryotic.

Mammalian cell lines available as hosts for expression are well known in the art and include, but are not limited to, immortalized cell lines available from the American Type Culture Collection (ATCC) and any cell lines used in an expression system known in the art can be used to make polypeptides comprising the engineered immunoglobulins (e.g., engineered antibodies) or fragments thereof of the present disclosure. In general, host cells are transformed with a recombinant expression vector that comprises DNA encoding a desired engineered immunoglobulin. Among the host cells that may be employed are prokaryotes, yeast or higher eukaryotic cells. Prokaryotes include gram negative or gram-positive organisms, for example E. coli or bacilli. Higher eukaryotic cells include insect cells and established cell lines of mammalian origin. Examples of suitable mammalian host cell lines include the COS-7 cells, L cells, C127 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells.

### Pharmaceutical Compositions

Provided herein are pharmaceutical compositions comprising the engineered immunoglobulin (e.g. engineered antibody) or fragment thereof of present disclosure. The engineered immunoglobulin can be in combination with one or more pharmaceutically acceptable excipients, diluents or carriers.

To prepare pharmaceutical or sterile compositions comprising an engineered immunoglobulin of the present disclosure, the immunoglobulin may be mixed with a pharmaceutically acceptable excipient(s), diluent(s) or carrier(s). In one embodiment, the pharmaceutical composition of present disclosure is combination with one or more pharmaceutically acceptable excipients, diluents or carriers. The phrase “pharmaceutically acceptable” means approved by a regulatory agency of a federal or a state government, or listed in the U.S. Pharmacopeia or other generally recognized pharmacopeia for use in animals, and more particularly, in humans. The term “pharmaceutical composition” refers to a mixture of at least one active ingredient (e.g., an engineered immunoglobulin of the present disclosure) and at least one pharmaceutically-acceptable excipient, diluent or carrier. A “medicament” refers to a substance used for medical treatment.

Pharmaceutical compositions of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Oral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

In one embodiment, the pharmaceutical composition of present disclosure an therapeutically effective amount of an engineered immunoglobulin or fragment thereof of the present disclosure. As used herein, the terms “effective amount” or “therapeutically effective amount” refer to an amount of a therapy (e.g. an engineered antibody) which is sufficient to reduce and/or ameliorate the severity and/or duration of a given condition, disorder, or disease and/or a symptom related thereto. These terms also encompass an amount necessary for the reduction, slowing, or amelioration of the advancement or progression of a given condition, disorder, or disease, reduction, slowing, or amelioration of the recurrence, development or onset of a given condition, disorder or disease, and/or to improve or enhance the prophylactic or therapeutic effect(s) of another therapy.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Where necessary, the therapeutic comprising the engineered immunoglobulin of the present disclosure may be incorporated into a composition that includes a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and WO 92/19244, WO 97/32572, WO 97/44013, WO 98/31346, and WO 99/66903, each of which is incorporated herein by reference their entirety.

A therapeutic comprising an engineered immunoglobulin of the present disclosure can also be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually or topically.

The therapeutic comprising an engineered immunoglobulin of the present disclosure may be administered via any of the above routes using, e.g., an injection device, an injection pen, a vial and syringe, pre-filled syringe, auto injector, an infusion pump, a patch pump, an infusion bag and needle, etc. If the molecules or fragments thereof of the disclosure are administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas (1983) J. Macromol. Sci. Rev. Macromol. Chem. 23:61; see also Levy et al., (1985) Science 228:190; During et al., (1989) Ann. Neurol. 25:351; Howard et al., (1989) J. Neurosurg., 7(1):105; U.S. Pat. Nos. 5,679,377; 5,916,597; 5,912,015; 5,989,463; 5,128,326; WO 99/15154; and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (Science (1990) 249:1527-1533). Any technique known to one of skill in the art can be used to produce sustained release formulations comprising one or more molecules or fragments thereof of the present application. See, e.g., U.S. Pat. No. 4,526,938, WO 91/05548, WO 96/20698, Ning et al., (1996) Radiotherapy & Oncology 39: 179-189; Song et al., (1995) PDA Journal of Pharm Sci & Tech., 50: 372-397; Cleek et al., (1997) Pro. Int'l. Symp. Control. Rel. Bioact. Mater. 24: 853-854; Lam et al., (1997) Proc. Int'l. Symp. Control Rel. Bioact. Mater., 24: 759-760, each of which is incorporated herein by reference in their entirety.

If a pharmaceutical composition comprising an engineered immunoglobulin of the present disclosure is administered topically, it can be formulated in the form of an ointment, cream, transdermal patch, lotion, gel, shampoo, spray, aerosol, solution, emulsion, or other forms known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as Freon) or in a squeeze bottle. Moisturizers or humectants can also be added to pharmaceutical compositions and dosage forms if desired. Examples of such additional ingredients are known in the art.

If a pharmaceutical composition comprising an engineered immunoglobulin of the present disclosure is administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

A pharmaceutical composition comprising an engineered immunoglobulin of the present disclosure can also be cyclically administered to a patient.

In certain embodiments, pharmaceutical compositions comprising an engineered immunoglobulin of the present disclosure can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade V V (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (P. G. Bloeman et al., (1995) FEBS Lett., 357: 140; M. Owais et al. (1995) Antimicrob. Agents Chemother., 39: 180); surfactant protein A receptor (Briscoe et al., (1995) Am. J. Physiol. 1233:134); p 120 (Schreier et al (1994) J. Biol. Chem. 269:9090); see also Keinänen & Laukkanen (1994) FEBS Lett., 346:123-6; Killion & Fidler (1994) Immunomethods, 4: 273.

In some embodiments, a pharmaceutical composition of the disclosure further comprises one or more additional therapeutic agents.

### EXAMPLES

Engineered immunoglobulins and Fc fragments were expressed, purified and analyzed, and the results are shown in Example 1.

Thermostability is a crucial pharmaceutical property in the development of therapeutic antibodies. Lower thermal stability of a product can result in a less stable product and for instance yield higher degree of aggregation, whereas higher thermal stability of a product could in principle decrease the extent of aggregation. The thermal stability of engineered immunoglobulins and their parental IgG were compared using a calorimetric measurement, e.g., a differential scanning micro calorimeter (Nano DSC, TA Instrument), which detects changes in the heat capacity of a protein solution upon unfolding. The results of the calorimetric measurements are shown in Example 2.

The binding affinities of engineered immunoglobulins and Fc fragments to human Fc receptors were determined. The technique used to measure the binding affinity is surface plasmon resonance (SPR) spectroscopy, a label-free technique which enables measurement of real-time ligand-binding affinities and kinetics using relatively small amounts of membrane protein in a native or native-like environment. A direct binding assay was performed to characterize the binding of the engineered immunoglobulins against hFcγR1a, hFcγR2a, hFcγR3a (F158V), hC1q or hFcRn, and the results are shown in Example 3. In addition, a direct binding assay was performed to determine the impact of described engineering on the binding of the engineered anti-CD3 immunoglobulin to hCD3epsilon antigen, and the results are shown in Example 4.

Activating Fcγ receptors (FcγRs) play a critical role in ADCC. Antibodies bound to a cell-surface antigen interact with FcγRs expressed on effector cells such as natural killer (NK) cells, neutrophils and macrophages, inducing these cells to exert cytotoxicity. In order to monitor if there is any activation of Jurkat/FcγR cells by the engineered immunoglobulins, Jurkat reporter gene assay (RGA) for the nuclear factor of activated T-cells (NFAT) pathway was performed using Jurkat NFAT luciferized (JNL) cells and THP-1 cells. The results are shown in Example 5.

The results on the biophysical properties of the engineered immunoglobulins and their binding affinities to human Fc receptors as well as the Fc effector functions are summarized in Table 9.

### Example 1: Expression and Purification of Engineered Immunoglobulins

Engineered immunoglobulins expressed, purified and analyzed following the procedure described below are presented in Table 3. Protein and corresponding nucleotide sequences are described Table 8.

Anti-CD3 monospecific IgG1, anti-CD3 monospecific IgG4 and anti-CD3xTargetAxTargetB trispecific IgG1 presented above were produced in HEK293T-17SF system. Nucleic acid sequences coding for heavy and light chains were synthesized at Geneart (LifeTechnologies) and cloned into a mammalian expression vector using restriction enzyme-ligation based cloning techniques. Plasmids encoding for heavy chain and light chain were co-transfected into HEK293T cells. In brief, for transient expression of immunoglobulins, equal quantities of light chain and each engineered heavy chain vectors were co-transfected into suspension-adapted HEK293T cells using Polyethylenimine ((PEI) Ref. cat #24765 Polysciences, Inc.). Typically, 100 ml of cells in suspension at a density of 1-2 Mio cells per ml was transfected with DNA containing 100 μg of expression vector encoding the engineered heavy chain and expression vector encoding the light chain, using 1:1 HC:LC ratio. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 μg/ml antibiotic.

Anti-TargetC monospecific antibodies were produced using the Novartis-proprietary Chinese hamster ovary cell line (CHO-C8TD) manufacturing expression system. Plasmids encoding for heavy chain and light chain were transfected into 5.0×10⁶ viable cells in 100 μl of cell culture medium. The transfected cells were seeded into 20 ml of cell culture medium with low concentration of folic acid in 125 ml shake flasks. Cells were grown in a humidified shaker incubator (orbital throw of 50 mm diameter) at 150 rpm, at 36.5° C. and 10% CO2. On day 3 post transfection, MTX was added to the culture at a final concentration of 10 nM to start selection for stable transfectants. The cells went into a selection crisis and recovered within 21 days. Then vials of the selected stable pools were frozen. For production of engineered anti-TargetC immunoglobulins, a fed-batch approach was used. A vial of the frozen cells was thawed. After recovery from thawing, cells were seeded into 100 ml of Novartis-proprietary production cell culture medium in 500 ml shake flasks. Cultures were grown in a humidified shaker incubator (orbital throw of 50 mm diameter) at 200 rpm, at 36.5° C. and 10% CO2. Growth temperature was decreased to 33° C. on day 5 after seeding the culture. Novartis-proprietary feed solutions were added on day 3, 4, 5, 6, 7 and 10 after seeding. The culture was harvested on day 11 after seeding. Cells were separated from the cell culture medium by centrifugation and sterile filtering.

The produced constructs were then purified from cell-free supernatant using immuno-affinity chromatography. Protein A resin (CaptivA PrimAb™, Repligen), equilibrated with PBS buffer pH 7.4 was incubated with filtered conditioned media using liquid chromatography system (Aekta pure chromatography system, GE Healthcare Life Sciences). The resin was washed with PBS pH 7.4 before the constructs were eluted with elution buffer (50 mM citrate, 90 mM NaCl, pH 2.7).

After capture, eluted proteins were pH neutralized using 1M TRIS pH 10.0 solution and polished using size exclusion chromatography technique (HiPrep Superdex 200 16/60, GE Healthcare Life Sciences).

Finally, engineered immunoglobulins were polished using size exclusion chromatography technique (HiPrep Superdex 200 16/60, GE Healthcare Life Sciences), using PBS pH7.4 as equilibration and elution buffer. Purified proteins were finally formulated in PBS buffer pH 7.4.

Aggregation propensity was measured after capture and pH neutralization step using analytical size exclusion chromatography technique (Superdex 200 Increase 3.2/300 GL, GE Healthcare Life Sciences).

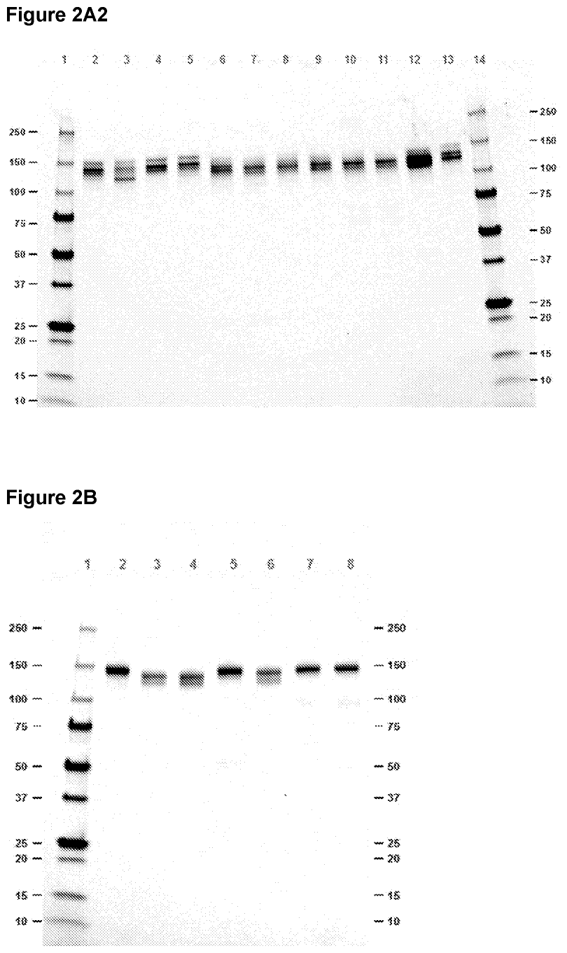

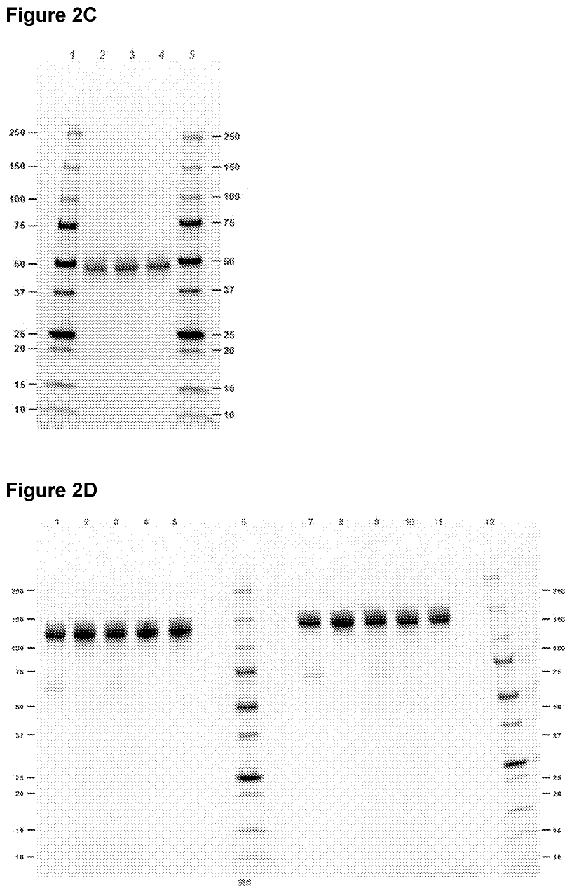

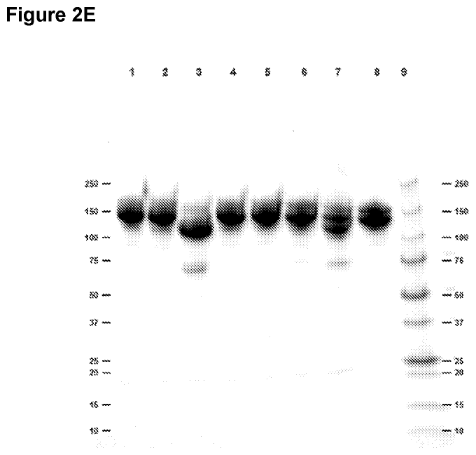

Purified immunoglobulins were further analyzed by SDS-PAGE (Sodium dodecyl sulfate polyacrylamide gel electrophoresis), where proteins are separated based on their molecular weight. Each protein was mixed with Laemmli buffer before loading on polyacrylamide gel (Biorad, 4-20% Mini-PROTEAN TGX Stain free). After 30 min migration at 200V in TRIS-Glycine-SDS running buffer, proteins contained in the gel were revealed in a stain-free enabled imager (Biorad, Gel Doc EZ). Those gels are shown FIG. 2.

FIG. 2A shows some of the produced engineered anti-CD3 monospecific IgG1 under non-reducing conditions (FIG. 2A1 and FIG. 2A2):

FIG. 2A1

| Line 1, 14: | Molecular weight marker (Biorad, Precision plus protein) | |
|---|---|---|
| Line 2: | CD3_WT | ~145 kDa |
| Line 3: | CD3_WT_YTE | ~145 kDa |
| Line 4: | CD3_1 | ~145 kDa |
| Line 5: | CD3_1_YTE | ~145 kDa |
| Line 6: | CD3_2 | ~145 kDa |
| Line 7: | CD3_2_YTE | ~145 kDa |
| Line 8: | CD3_5 | ~145 kDa |
| Line 9: | CD3_5_YTE | ~145 kDa |
| Line 10: | CD3_6 | ~145 kDa |
| Line 11: | CD3_6_YTE | ~145 kDa |
| Line 12: | CD3_7 | ~145 kDa |
| Line 13: | CD3_7_YTE | ~145 kDa |

FIG. 2A2

| Line 1, 14: | Molecular weight marker (Biorad, Precision plus protein) | |
|---|---|---|
| Line 2: | CD3_8 | ~145 kDa |
| Line 3: | CD3_8_YTE | ~145 kDa |
| Line 4: | CD3_9 | ~145 kDa |
| Line 5: | CD3_9_YTE | ~145 kDa |
| Line 6: | CD3_10 | ~145 kDa |
| Line 7: | CD3_10_YTE | ~145 kDa |
| Line 8: | CD3_11 | ~145 kDa |
| Line 9: | CD3_11_YTE | ~145 kDa |
| Line 10: | CD3_12 | ~145 kDa |
| Line 11: | CD3_12_YTE | ~145 kDa |
| Line 12: | CD3_DANAPA | ~145 kDa |
| Line 13: | CD3_DANAPA_YTE | ~145 kDa |

FIG. 2B shows some of the produced engineered anti-CD3xTargetAx TargetB trispecific IgG1 under non-reducing conditions:

FIG. 2B

| Line 1: | Molecular weight marker (Biorad, Precision plus protein) | |
|---|---|---|
| Line 2: | CD3 × TargetA × TargetB | ~140 kDa |
| Line 3: | CD3 × TargetA × TargetB_1 | ~140 kDa |
| Line 4: | CD3 × TargetA × TargetB_2 | ~140 kDa |
| Line 5: | CD3 × TargetA × TargetB_6 | ~140 kDa |
| Line 6: | CD3 × TargetA × TargetB_7 | ~140 kDa |
| Line 7: | CD3 × TargetA × TargetB_8 | ~140 kDa |
| Line 8: | CD3 × TargetA × TargetB_9 | ~140 kDa |

FIG. 2C shows some of the produced engineered IgG1 FC under non-reducing conditions:

FIG. 2C

| Line 1, 5: | Molecular weight marker (Biorad, Precision plus protein) | |
|---|---|---|
| Line 2: | IgG1_FC_6 | ~50 kDa |
| Line 3: | IgG1_FC_8 | ~50 kDa |
| Line 4: | IgG1_FC_9 | ~50 kDa |

FIG. 2D shows some of the produced engineered anti-CD3 monospecific IgG4 under non-reducing conditions:

FIG. 2D

FIG. 2D

| Line 6, 12: | Molecular weight marker (Biorad, Precision plus protein) | |
|---|---|---|
| Line 1: | IgG4_CD3_WT | ~145 kDa |
| Line 2: | IgG4_CD3_S228P | ~145 kDa |
| Line 3: | IgG4_CD3_6 | ~145 kDa |
| Line 4: | IgG4_CD3_8 | ~145 kDa |
| Line 5: | IgG4_CD3_9 | ~145 kDa |
| Line 7: | IgG4_CD3_WT_YTE | ~145 kDa |
| Line 8: | IgG4_CD3_S228P_YTE | ~145 kDa |
| Line 9: | IgG4_CD3_6_YTE | ~145 kDa |
| Line 10: | IgG4_CD3_8_YTE | ~145 kDa |
| Line 11: | IgG4_CD3_9_YTE | ~145 kDa |

FIG. 2E shows some of the produced engineered anti-TargetC monospecific IgG1 under non-reducing conditions:

FIG. 2E

| Line 9: | Molecular weight marker (Biorad, Precision plus protein) | |
|---|---|---|
| Line 1: | TargetC_WT | ~145 kDa |
| Line 2: | TargetC_6 | ~145 kDa |
| Line 3: | TargetC_7 | ~145 kDa |
| Line 4: | TargetC_8 | ~145 kDa |
| Line 5: | TargetC_9 | ~145 kDa |
| Line 6: | TargetC_10 | ~145 kDa |
| Line 7: | TargetC_11 | ~145 kDa |
| Line 8: | TargetC_DANAPA | ~145 kDa |

Results of expression yield following 2-step purification are presented Table 4. Aggregation content after capture step of this immunoglobulin set is as well described Table 4.

TABLE 4
Expression yield after 2-step purification and aggregation content after capture.

| | Relative Expression yield after | |
|---|---|---|
| | 2-step purification (% of WT | Aggregation after capture |
| | final yield) | (%) |
| Anti-CD3 monospecific hIgG1 | | |
| CD3_WT | 100.0 | 14.8 |
| CD3_WT_YTE | 102.7 | 13.7 |
| CD3_1 | 96.1 | 14.8 |
| CD3_1_YTE | 97.6 | 16.0 |
| CD3_2 | 96.1 | 14.0 |
| CD3_2_YTE | 90.0 | 13.9 |
| CD3_3 | 104.7 | 14.4 |
| CD3_3_YTE | 102.7 | 14.2 |
| CD3_4 | 53.7 | 7.9 |
| CD3_4_YTE | 70.1 | 9.8 |
| CD3_5 | 73.8 | 16.7 |
| CD3_5_YTE | 64.5 | 15.8 |
| CD3_6 | 109.1 | 15.7 |
| CD3_6_YTE | 95.1 | 18.2 |
| CD3_7 | 70.5 | 18.0 |
| CD3_7_YTE | 67.1 | 14.4 |
| CD3_8 | 87.1 | 13.2 |
| CD3_8_YTE | 96.8 | 11.8 |
| CD3_9 | 101.6 | 12.5 |
| CD3_9_YTE | 96.8 | 12.9 |
| CD3_10 | 103.2 | 14.9 |
| CD3_10_YTE | 104.8 | 17.6 |
| CD3_11 | 96.8 | 17.1 |
| CD3_11_YTE | 103.2 | 16.6 |
| CD3_12 | 69.1 | Nd |
| CD3_12_YTE | 63.3 | Nd |
| CD3_DANAPA | 80.0 | 9.8 |
| CD3_DANAPA_YTE | 67.6 | 9.1 |
| Anti-TargetC monospecific | | |
| hIgG1 | | |
| TargetC_WT | 100.0 | 1.1 |
| TargetC_6 | 100.0 | 0.7 |
| TargetC_7 | 104.2 | 2.3 |
| TargetC_8 | 95.8 | 0.8 |
| TargetC_9 | 100.0 | 0.5 |
| TargetC_10 | 112.5 | 2.2 |
| TargetC_11 | 108.3 | 6.6 |
| TargetC_DANAPA | 95.8 | 2.8 |
| Anti-CD3 × TargetA × TargetB | | |
| trispecific hIgG1 | | |
| CD3 × TargetA × TargetB_WT | 100.0 | 10.8 |
| CD3 × TargetA × TargetB_DANAPA | 53.5 | 22.7 |
| CD3 × TargetA × TargetB_1 | 86.8 | 15.4 |
| CD3 × TargetA × TargetB_2 | 66.7 | 13.5 |
| CD3 × TargetA × TargetB_6 | 117.5 | 10.0 |
| CD3 × TargetA × TargetB_7 | 82.0 | 12.6 |
| CD3 × TargetA × TargetB_8 | 97.4 | 13.2 |
| CD3 × TargetA × TargetB_9 | 115.4 | 9.7 |
| Anti-CD3 monospecific hIgG4 | | |
| IgG4_CD3_WT | 100.0 | 6.0 |
| IgG4_CD3_WT_YTE | 65.1 | 6.0 |
| IgG4_CD3_S228P | 103.8 | 4.3 |
| IgG4_CD3_S228P_YTE | 79.2 | 5.5 |
| IgG4_CD3_6 | 89.6 | 6.5 |
| IgG4_CD3_6_YTE | 68.9 | 6.8 |
| IgG4_CD3_8 | 94.3 | 4.5 |
| IgG4_CD3_8_YTE | 106.6 | 5.2 |
| IgG4_CD3_9 | 102.8 | 5.0 |
| IgG4_CD3_9_YTE | 88.7 | 5.3 |

Nd: not determined

As shown in the results of Table 4, transferring IgA top CH2 structural element into hIgG1 FC does not affect dramatically expression yield, nor aggregation propensity of these molecules. In fact, such engineered molecules keep expression yield and aggregation propensity being in the same range as observed with parental hIgG1.

Moreover, described mutation sets are compatible with YTE mutation set used for half-life extension. Those engineering molecules wearing additional YTE mutations set have expression yield and aggregation propensity being in the same range as observed with parental hIgG1 wearing same YTE mutations.

In addition, previous results exemplify how transferring of IgA top CH2 structural element into hIgG1 FC can be applied to different immunoglobulin formats. In fact, such engineering is translatable from monospecific to multispecific IgG1 (i.e., bi- and tri-specific) and is compatible with technologies used to direct HC/HC pairing (i.e., “Knob into hole” mutation set), allowing production of such engineered multispecific antibodies.

Finally, data show it is possible to translate such engineering from IgG1 to IgG4 isotype, with or without YTE mutation set. Similar to previous conclusions, such engineered molecules keep expression yield and aggregation propensity being in the same range as observed with parental hIgG4. Moreover, described mutations set constitute an alternative to S228P to prevent IgG4 Fab arm exchange.

### Example 2: Thermo-Stability Assessment of Engineered Immunoglobulins by Differential Scanning Calorimetry (DSC)

The thermal stability of parental and engineered immunoglobulins was measured using calorimetric measurements, as described below.

Calorimetric measurements were carried out on a differential scanning micro calorimeter (Nano DSC, TA Instrument or MicroCal, Malvern). The heating rate was 1° C./min. All proteins were used at a concentration of 1 mg/ml in PBS (pH 7.4). The heat capacity and molar heat capacity of each protein was estimated by comparison with duplicate samples containing identical buffer from which the protein had been omitted. Heat capacities, molar heat capacities and melting curves were analyzed using standard procedure. Thermograms were baseline corrected and concentration normalized.

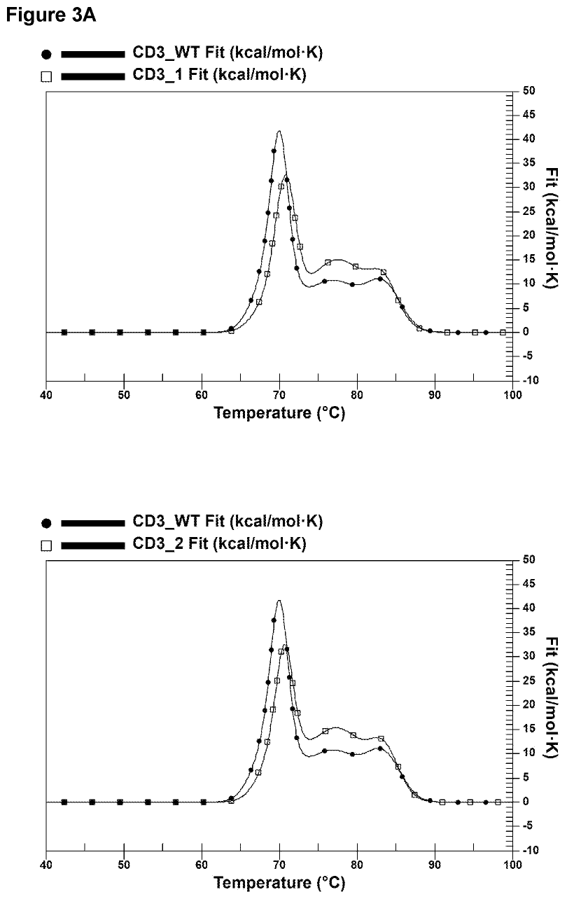

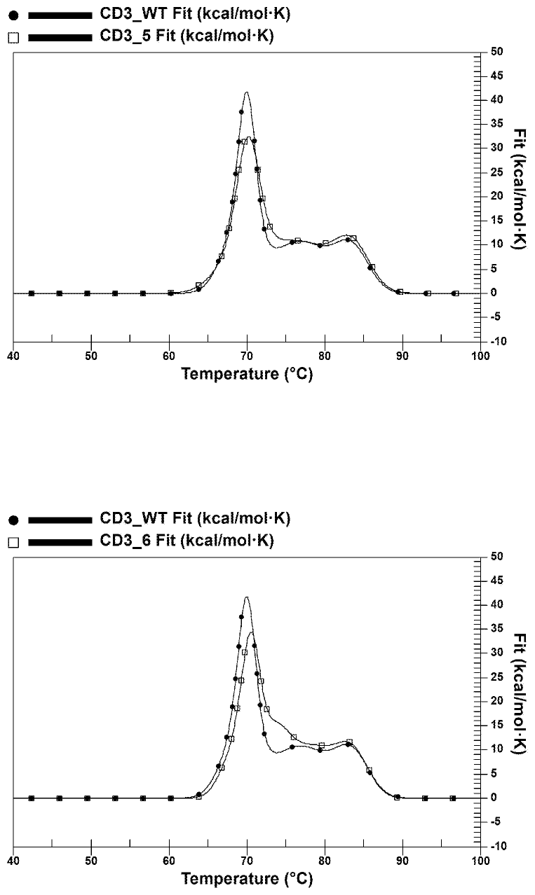

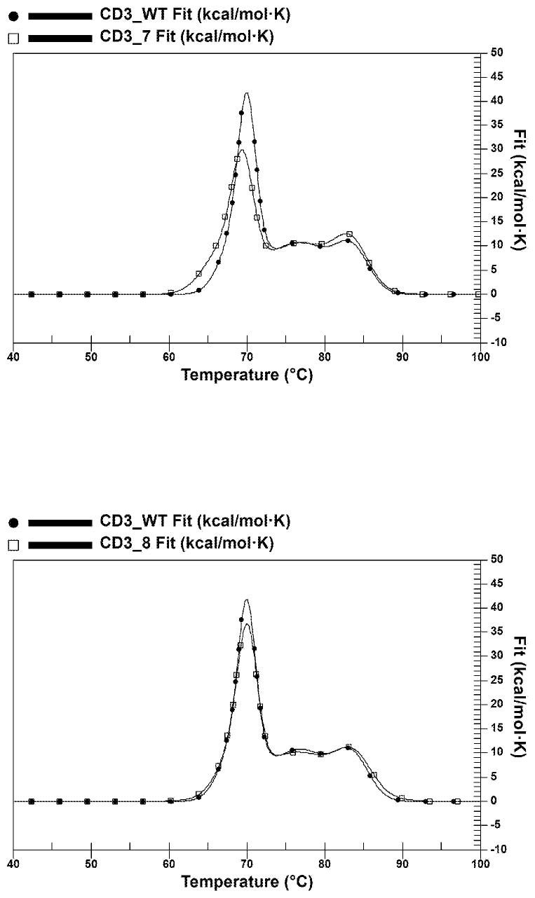

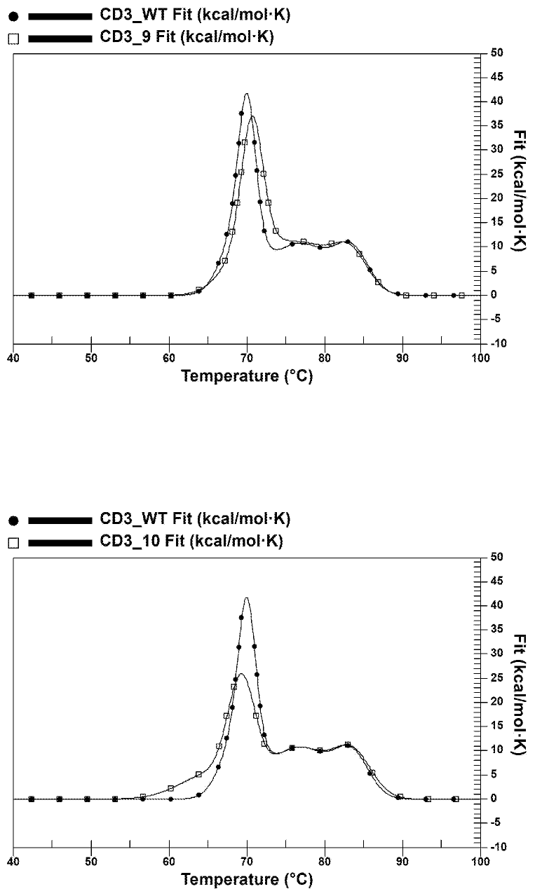

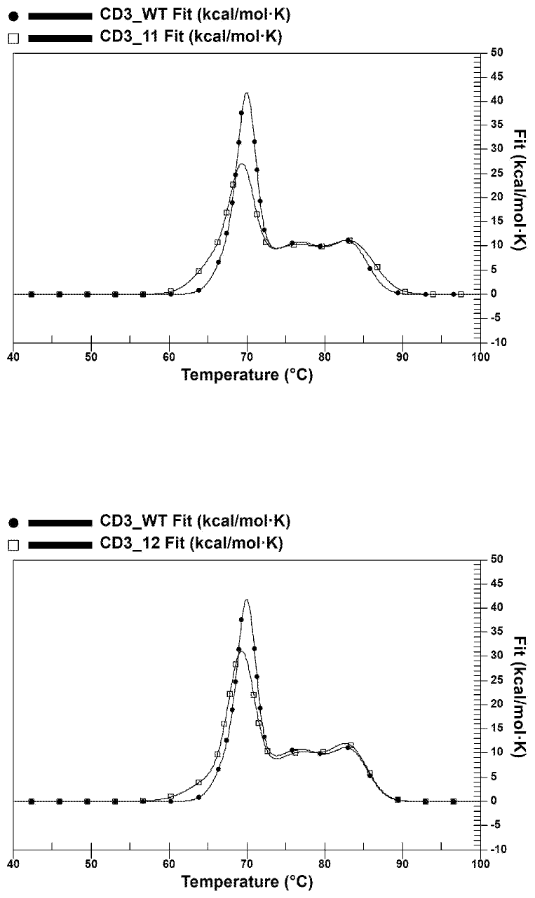

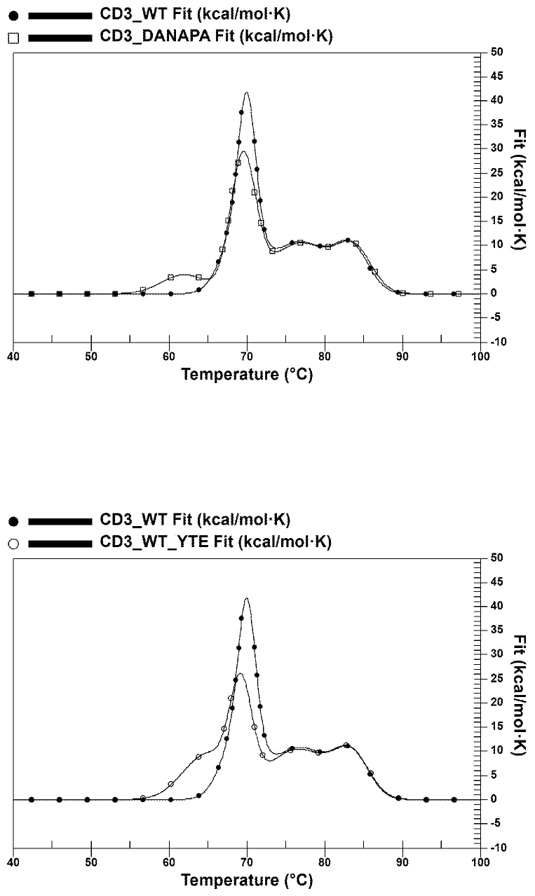

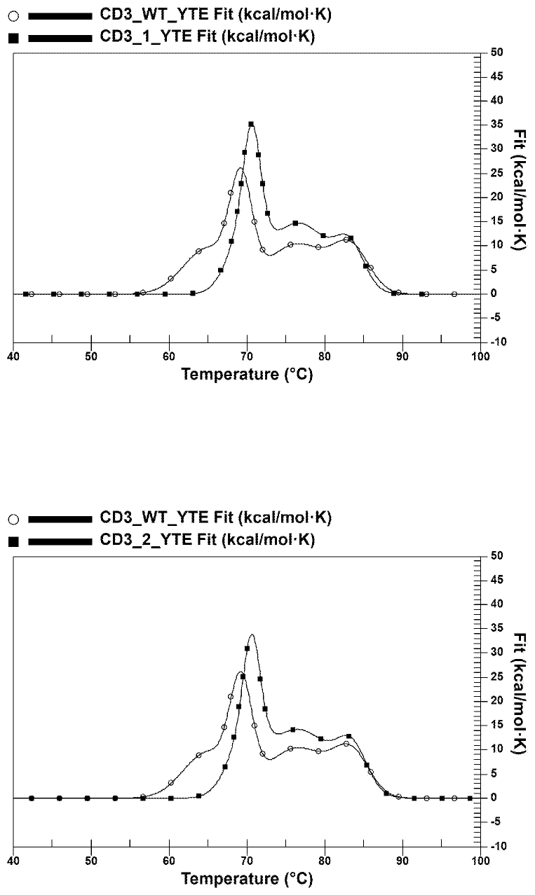

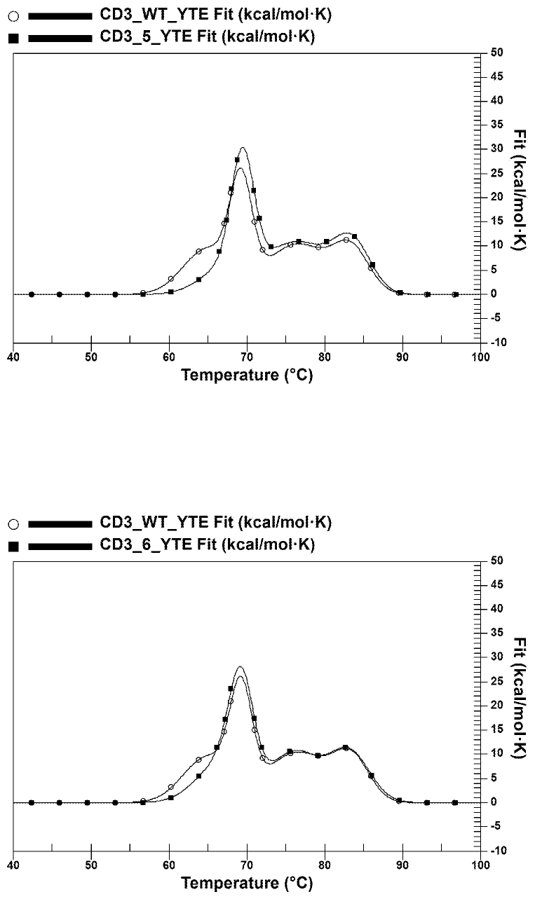

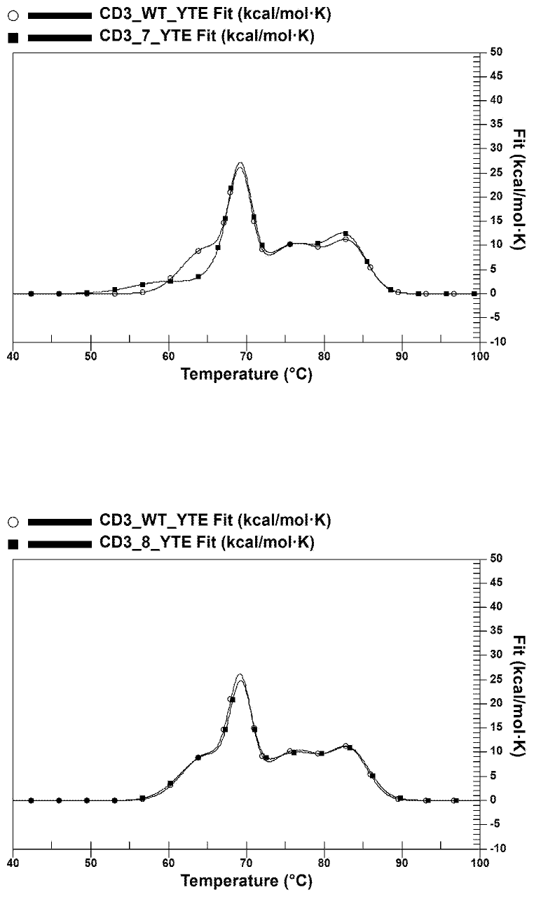

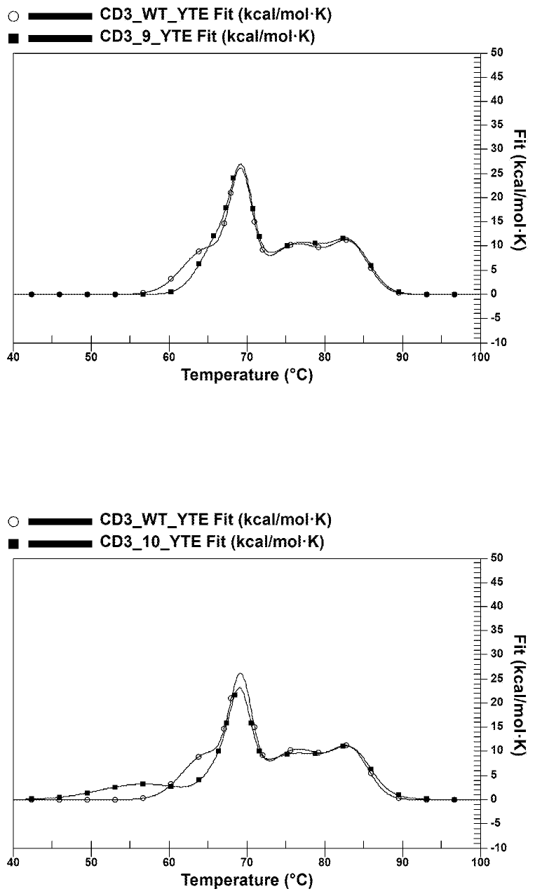

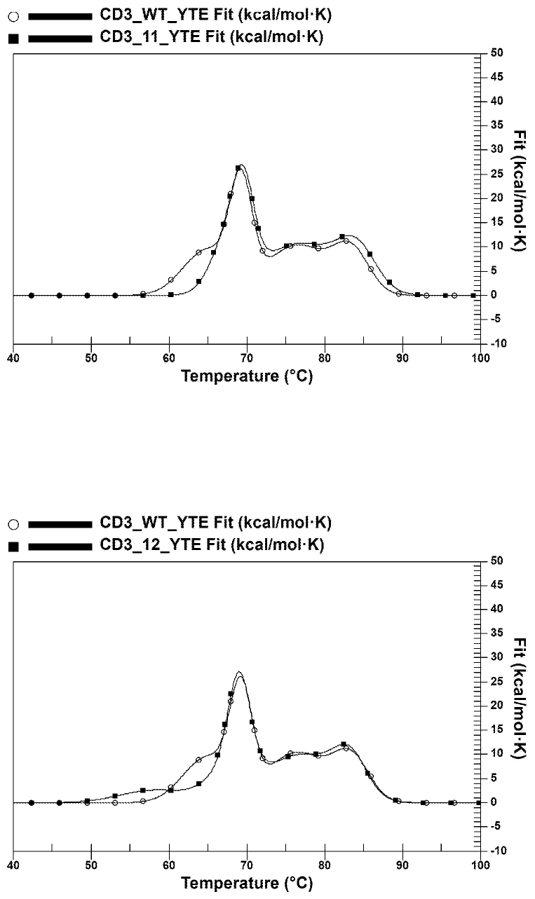

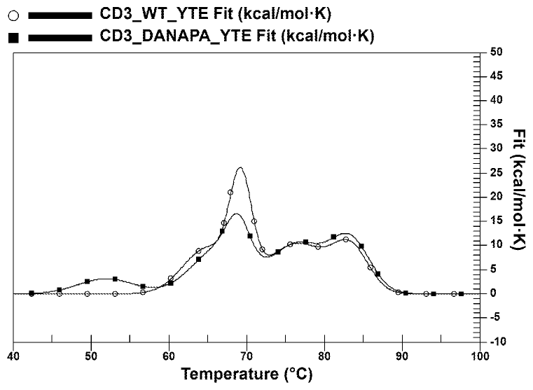

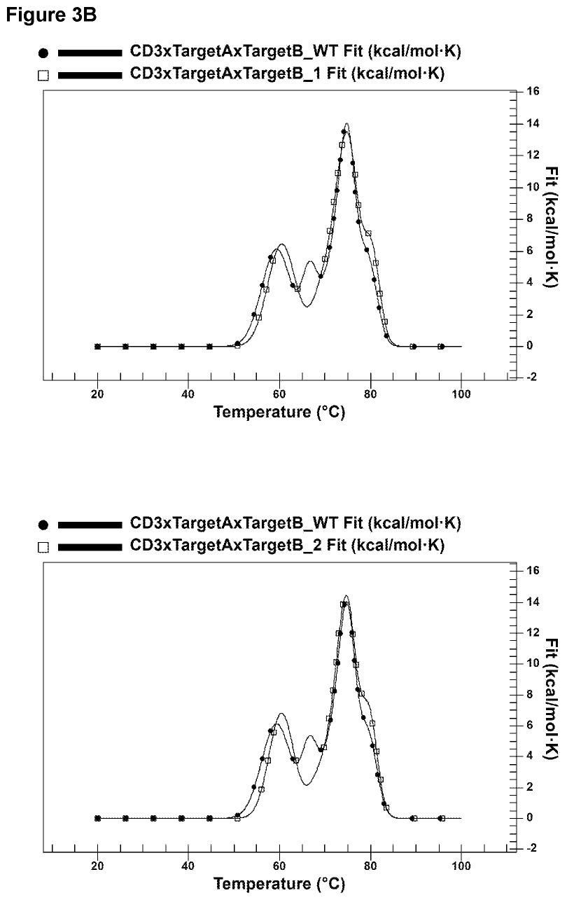

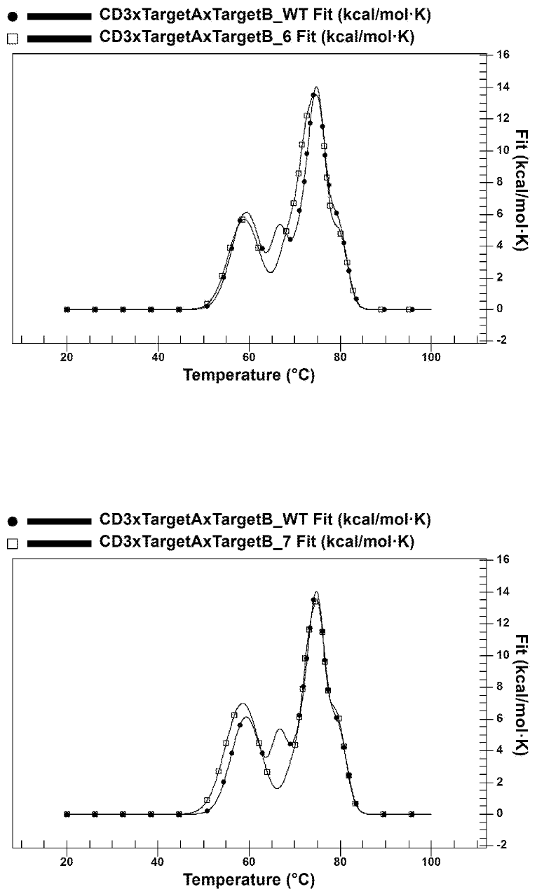

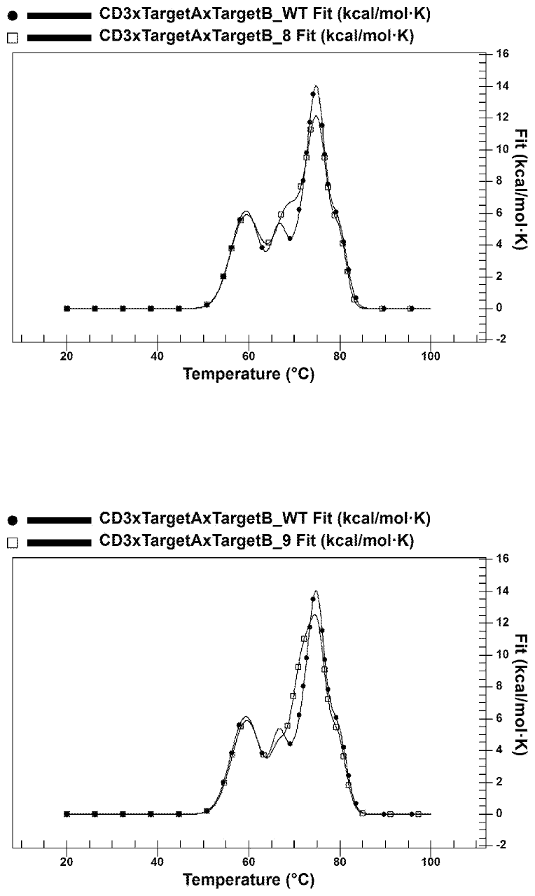

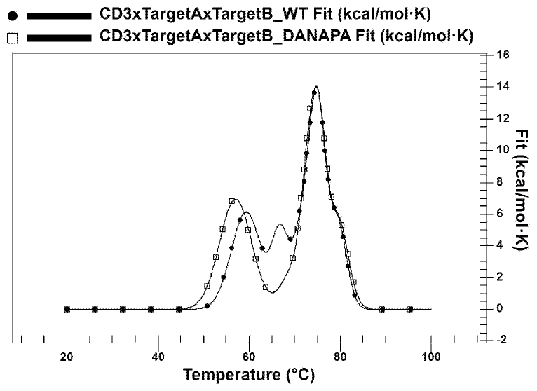

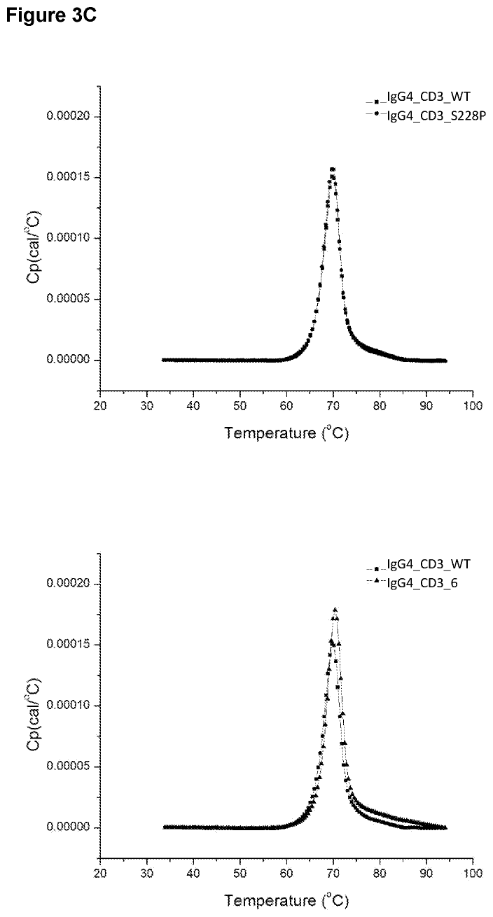

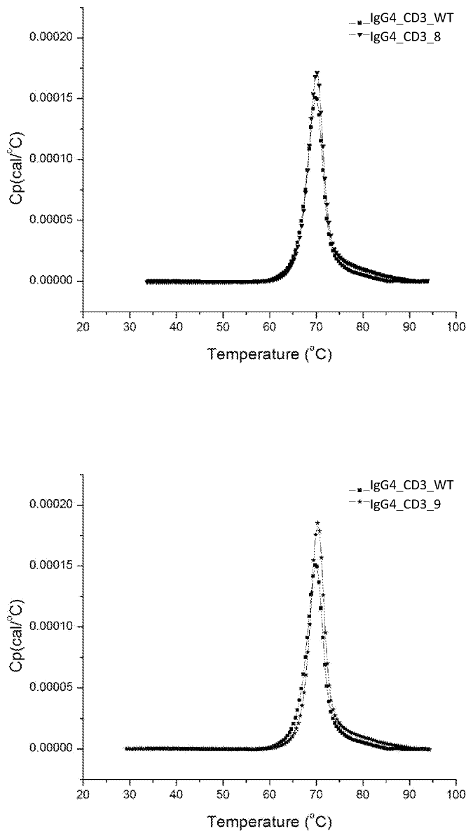

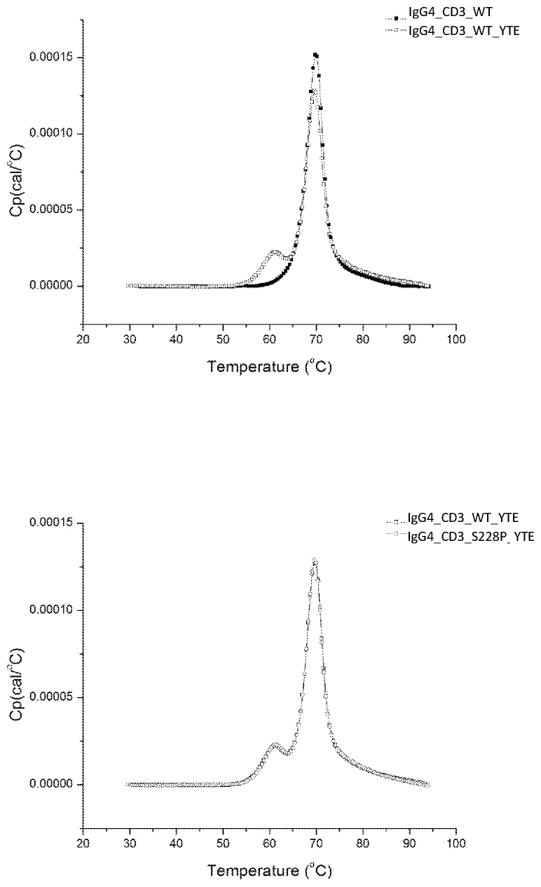

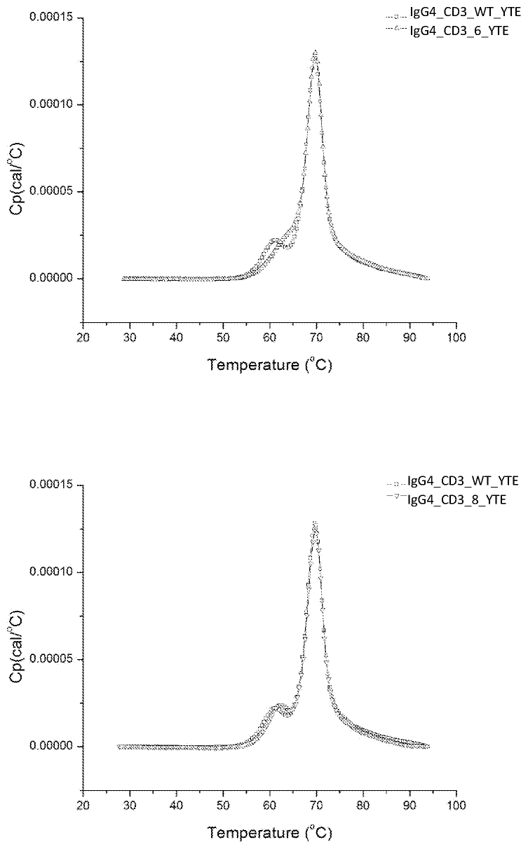

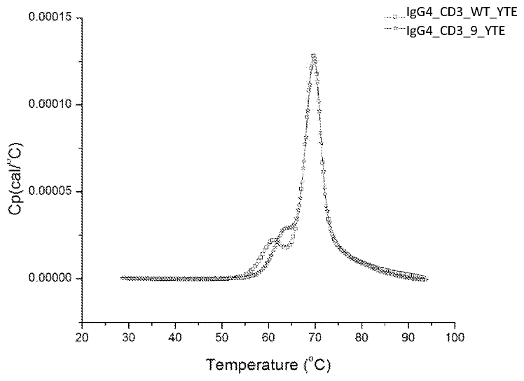

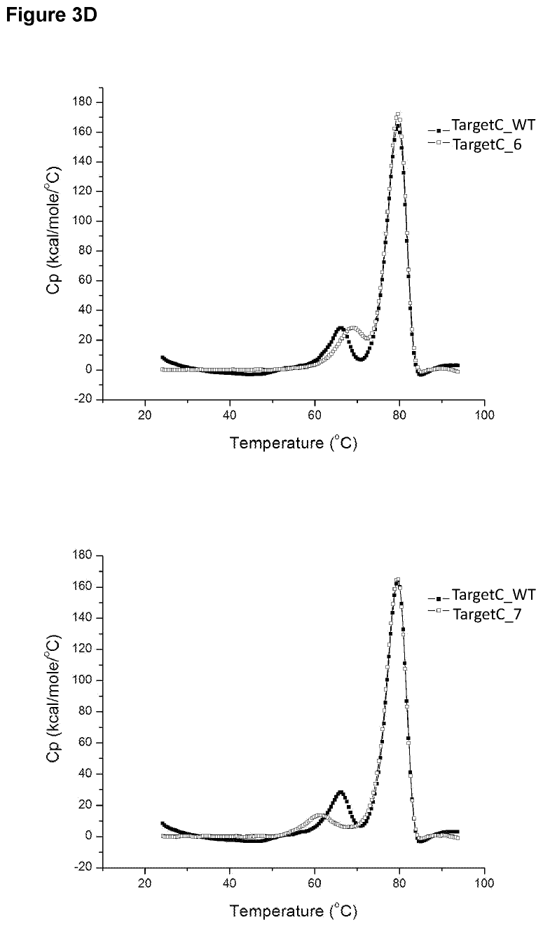

FIG. 3 shows overall thermal stability of the engineered immunoglobulins compare to parental one. Data demonstrates that transferring IgA top CH2 structural element into hIgG1 FC results in a more stable molecule with improved thermal stability over parental immunoglobulins. FIG. 3A and FIG. 3B show data obtained with Nano DSC, TA instrument. FIG. 3C and FIG. 3D present data obtained with MicroCal, Malvern instrument.

FIG. 3A describes overall thermal stability measurement done on engineered anti-CD3 monospecific hIgG1. Such improvement brought by described engineering can be clearly observed when comparing CD3_WT and CD3_1 immunoglobulins for instance. Beneficial effect is even more pronounced when stabilizing engineering is combined with YTE mutation set. Whereas YTE mutation set is destabilizing immunoglobulin, as shown when CD3_WT is compared to CD3_WT_YTE, introduction of IgA top CH2 structural element into IgG1 FC fully compensate this loss in thermo-stability. This is shown when comparing CD3_WT_YTE with CD3_1_YTE for instance. Interestingly, the more extensive engineering, the higher thermal stability improvement. Similar CH2 thermo-stabilization is observed when described engineering is applied to anti-TargetC monospecific hIgG1, as shown FIG. 3D.

As described FIG. 3B, same observations are done when applying such engineering to anti-CD3xTargetAxTargetB trispecific hIgG1. Thermal stability improvement brought by described engineering can be observed when comparing CD3xTargetAxTargetB_WT and CD3xTargetAxTargetB_1 immunoglobulins for instance.

FIG. 3C describes how thermal stability is improved when anti-CD3 monospecific hIgG4 is engineered.

Taken together these data show how CH2 thermostability of an immunoglobulin can be improved by applying the Fc engineering as described the present disclosure. Such stabilizing effect can be observed when protein engineering is applied to monospecific or multispecific IgG1 Fc or other isotype such as IgG4 for instance, wearing or not other mutation sets used for half-life modulation (i.e. YTE mutation set) or Fc heterodimerization (i.e. Knob into hole mutation set). Finally, engineered recombinant antibodies exhibit same CH2 thermo-stabilized profile whether they are produced in HEK293 or CHO expression system.

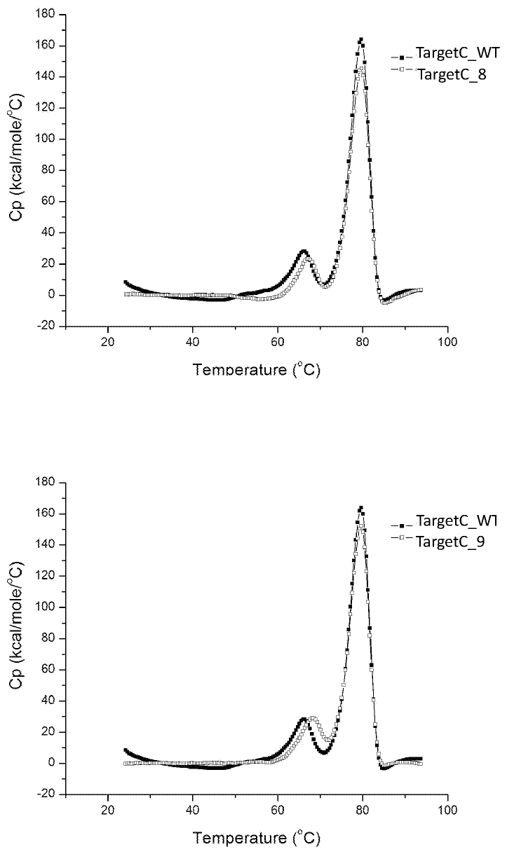

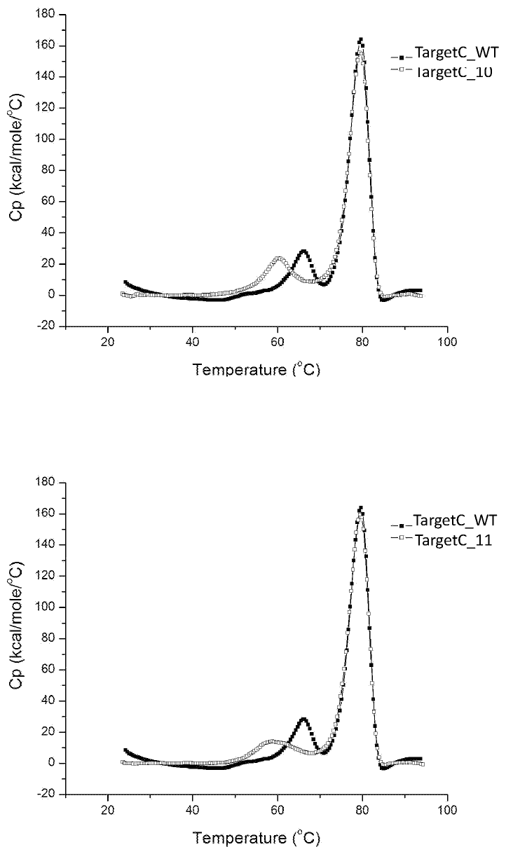

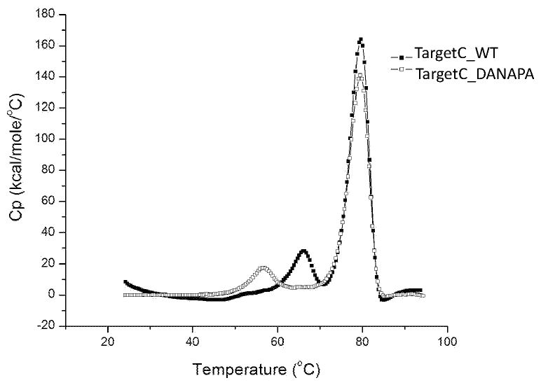

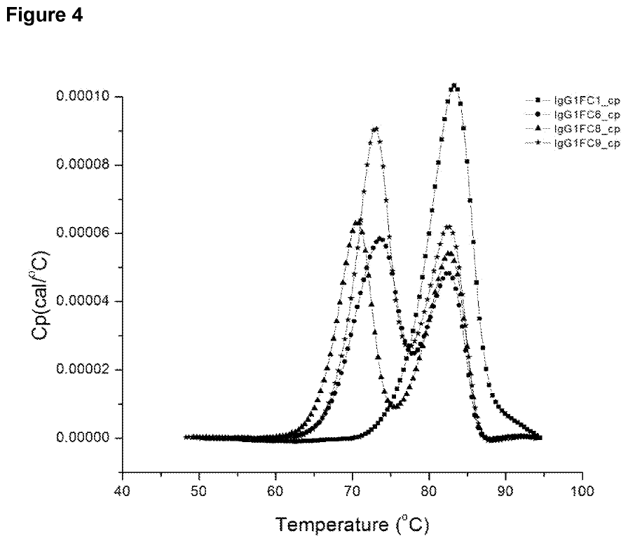

Corresponding recombinant Fcs were generated and some of them were used to characterize their thermal stability (FIG. 4), independently of the Fab. Melting temperature (TM) of CH2 and CH3 domains could be determined (Table 5) using MicroCal, Malvern instrument. Measurement done on IgG1_FC_1 for instance shows how strong the CH2 stabilization by introduction of IgA CH2 structural element into IgG1 is. In fact, CH2 TM is improved by 13° C., passing from 70.0° C. (Ionescu et al, 2007, Contribution of variable domains to the stability of humanized IgG1 monoclonal antibodies) to 83° C. Interestingly, thermal stability improvement is proportional to the number of IgA residues and number of additional disulfide bonds introduced into IgG1 FC. The more extensive engineering, the higher thermal stability improvement.

TABLE 5
Melting temperature (TM) of CH2 and CH3 domains measured
on Fc constructs, free of Fab fragment. Variations of TM
compared to parental IgG1 Fc are shown in parenthesis.

| ID | CH2 TM (° C.) | CH3 TM (° C.) |
|---|---|---|
| IgG1 FC WT (reported from literature) | 70.0 | 82.0 |
| IgG1_Fc_1 | 83.3 (+13.3) | 83.3 |
| IgG1_Fc_6 | 73.6 (+3.6) | 82.7 |
| IgG1_Fc_8 | 70.8 (+0.8) | 82.5 |
| IgG1_Fc_9 | 73.2 (+3.2) | 82.7 |

### Example 3: SPR Measurement Against Human Fc Receptors

The binding affinities of engineered immunoglobulins or fragments thereof to human Fc receptors were determined using surface plasmon resonance (SPR) spectroscopy. SPR is a technology generally applied to affinity and kinetic analysis of protein-protein, protein-peptide, protein-DNA, and protein-small molecule interactions, as it allows the analysis of interactions between analytes in solution and a ligand attached to a sensor chip surface, providing a continuous readout of complex formation and dissociation.

A direct binding assay was performed to characterize the binding of the engineered immunoglobulins against hFcγR1a, hFcγR2a, hFcγR3a (F158V), hC1q or hFcRn.

Kinetics and binding capacity were measured on a BIAcore® T200 instrument (GE Healthcare, Glattbrugg, Switzerland) at room temperature, with proteins diluted in running buffer 10 mM NaP, 150 mM NaCl, 0.05% Tween 20, pH7.6. A CM5 sensor chip (Sensor Chip SA, GE Healthcare Life Sciences) was used to immobilized engineered immunoglobulins by amine coupling. Then, recombinant human hFcγR1a, or recombinant human hFcγR3a (F158V), or recombinant human hFcRn, or hC1q was used as the analyte.

To serve as a reference, one flow cell did not receive any immunoglobulin, and was deactivated using Ethanolamine. Binding data were acquired by subsequent injection of analyte dilution series on the reference and measuring flow cells. Zero concentration samples (running buffer only) were included to allow double referencing during data evaluation. For data evaluation, doubled referenced sensorgrams were analyzed and the maximum response reached during the experiment was monitored. Maximum response describes the binding capacity of the surface in terms of the response at saturation. Finally, measured maximum responses were normalized to the one measured using parental immunoglobulin (not engineered). Concerning anti-TargetC monospecific antibodies, affinity (KD) for hFcRn at pH5.8 was determined. Results are shown Table 6.

TABLE 6
Maximum response of engineered immunoglobulin toward hFcγR1a,
hFcγR2a, hFcγR3a (F158V), hC1q or hFcRn

| | Maximum | | Maximum | | |
|---|---|---|---|---|---|
| | response at | Maximum | response at | Maximum | Maximum |
| | 100 nM | response for | 300 nM | response at | response at |
| Anti-CD3 monospecific | hFcγR1a | hFcγR2a | hFcγR3a | 20 nM hC1q | 1840 nM |
| hIgG1 | (RU) | (RU) | F158V (RU) | (RU) | hFcRn (RU) |
| CD3_WT | 100 | Nd | 100 | 100 | 100 |
| CD3_WT_YTE | 97 | Nd | 71 | Nd | 586 |
| CD3_1 | 2 | Nd | 10 | 5 | Nd |
| CD3_1_YTE | 6 | Nd | 20 | Nd | 290 |
| CD3_2 | 5 | Nd | 20 | 7 | Nd |
| CD3_2_YTE | 3 | Nd | 0 | Nd | 340 |
| CD3_3 | 3 | Nd | 2 | 7 | Nd |
| CD3_3_YTE | 7 | Nd | 10 | Nd | Nd |
| CD3_4 | 4 | Nd | 10 | 9 | Nd |
| CD3_4_YTE | 4 | Nd | 10 | Nd | Nd |
| CD3_5 | 7 | Nd | 22 | 0 | Nd |
| CD3_5_YTE | 7 | Nd | 17 | Nd | 340 |
| CD3_6 | 13 | Nd | 17 | 33 | 136 |
| CD3_6_YTE | 10 | Nd | 15 | Nd | 720 |
| CD3_7 | 21 | Nd | 5 | 2 | 80 |
| CD3_7_YTE | 31 | Nd | 19 | Nd | 284 |
| CD3_8 | 2 | Nd | Nd | 1 | 126 |
| CD3_8_YTE | 2 | Nd | Nd | 0 | 604 |
| CD3_9 | 2 | Nd | Nd | 10 | 122 |
| CD3_9_YTE | 2 | Nd | Nd | 4 | 696 |
| CD3_10 | 18 | Nd | Nd | 3 | 126 |
| CD3_10_YTE | 9 | Nd | Nd | 0 | 280 |
| CD3_11 | 19 | Nd | Nd | 0 | 92 |
| CD3_11_YTE | 12 | Nd | Nd | 1 | 230 |
| CD3_DANAPA | 6 | Nd | 10 | 1 | Nd |
| | Maximum | Maximum | Maximum | | |
| | response at | response at | response at | Maximum | KD for |
| | 20 nM | 4000 nM | 1000 nM | response at | hFcRn |
| Anti-TargetC | hFcγR1a | hFcγR2a | hFcγR3a | 200 nM | (nM) at |
| monospecific hIgG1 | (RU) | (RU) | F158V (RU) | hC1q (RU) | pH 5.8 |
| TargetC_WT | 100 | 100 | 100 | 100 | 1963 |
| TargetC_6 | 0 | 0 | 0 | 0 | 1711 |
| TargetC_7 | 18 | 0 | 0 | 0 | 2239 |
| TargetC_8 | 0 | 0 | 0 | 0 | 1814 |
| TargetC_9 | 0 | 0 | 0 | 4 | 1822 |
| TargetC_10 | 11 | 0 | 0 | 0 | 2200 |
| TargetC_11 | 27 | 0 | 0 | 3 | 2270 |
| TargetC_DANAPA | 0 | 0 | 0 | 0 | Nd |

Nd: not determined

Data demonstrate that introduction of IgA top CH2 structural element into IgG1 FC (mono- or multispecific) results in decrease of binding to Gamma receptors (FcγR1a, FcγR2a, FcγR3a, C1q for instance), while retaining reasonable binding to FcRn.

Use of YTE mutation set for half-life extension remains compatible with described stabilizing engineering. In fact, increase of binding level to FcRn could be shown with immunoglobulin having IgA top CH2 structural element combined with YTE mutation set.

### Example 4: SPR Measurement Against Human CD3epsilon

A direct binding assay was performed to determine the impact of described engineering on the binding of the engineered anti-CD3 immunoglobulin to hCD3epsilon antigen.

Kinetic binding affinity constants (KD) were measured measured on a BIAcore® T200 instrument (GE Healthcare, Glattbrugg, Switzerland) at room temperature, with proteins diluted in running buffer 10 mM NaP, 150 mM NaCl, 0.05% Tween 20, pH7.6. A CM5 sensor chip (Sensor Chip SA, GE Healthcare Life Sciences) was used to immobilize hCD3epsilon-FC antigen by amine coupling. Then, engineered anti-CD3 hIgG1 were used as the analyte.

To serve as a reference, one flow cell did not receive any antigen, and was deactivated using Ethanolamine. Binding data were acquired by subsequent injection of analyte dilution series on the reference and measuring flow cells. Zero concentration samples (running buffer only) were included to allow double referencing during data evaluation. For data evaluation, doubled referenced sensorgrams were analyzed by applying a 1:1 binding model analysis to generate the equilibrium dissociation constant (KD). Binding constant described Table 7 are considered as apparent KD since immunoglobulins used as analyte are bivalent for the immobilized antigen. In addition, maximum response reached during the experiment was monitored. Maximum response describes the binding capacity of the surface in terms of the response at saturation. Finally, measured maximum responses were normalized to the one measured using parental immunoglobulin (not engineered). Results presented Table 7 indicates that all engineered immunoglobulins bind the hCD3epsilon antigen similarly to their parental anti-CD3 IgG1 (CD3_WT).

TABLE 7
Affinity and maximum response of engineered anti-CD3 IgG1,
based on parental CD3_WT, toward hCD3epsilon antigen.

| Anti-CD3 monospecific hIgG1 | Apparent KD (M) | Maximum response at 125 nM hIgG1 |
|---|---|---|
| CD3_WT | 2.32E−09 | 392 |
| CD3_WT_YTE | 2.34E−09 | 342 |
| CD3_1 | 2.06E−09 | 371 |
| CD3_1_YTE | 2.09E−09 | 371 |
| CD3_2 | 2.24E−09 | 386 |
| CD3_2_YTE | 2.30E−09 | 371 |
| CD3_3 | 2.04E−09 | 375 |
| CD3_3_YTE | 2.10E−09 | 371 |
| CD3_4 | 3.38E−09 | 470 |
| CD3_4_YTE | 2.74E−09 | 427 |
| CD3_5 | 3.76E−09 | 365 |
| CD3_5_YTE | 3.74E−09 | 322 |
| CD3_6 | 2.38E−09 | 338 |
| CD3_6_YTE | 2.42E−09 | 340 |
| CD3_7 | 2.27E−09 | 367 |
| CD3_7_YTE | 2.31E−09 | 367 |
| CD3_DANAPA | 3.27E−09 | 392 |

Those results show introduction of IgA top CH2 structural element into an IgG1 Fc does not impact its antigen recognition by Fab.

### Example 5: Anti-CD3 NFAT Signaling Assay

Jurkat reporter gene assay (RGA) for the nuclear factor of activated T-cells (NFAT) pathway was performed using Jurkat NFAT luciferized (JNL) cells and THP-1 cells (ATCC, TIB202). THP-1 cells express Gamma receptors FcγRI, FcγRII, and FcγRIII and were pre-treated with 100 u/mL IFNg for 48 hours at 37° C., 5% CO₂ before co-culture. Cells were co-incubated for 6 hours at 37° C., 5% CO₂ at a 10:1 target to effector cell ratio with each sample at the various concentrations depicted. An equal volume of ONE-Glo™ reagent (Promega, E6120) was added to the culture volume. Plate was shaken for 2 minutes, then incubated for an additional 8 minutes protected from light. Luciferase activity was quantitated on the Biotek Synergy HT plate reader. Data were analyzed and fit to a 4 parameter-logistic curve using GraphPad Prism. NFAT activity directly translate the ability of the tested immunoglobulin to cross-link Jurkat and THP-1 cells. Finally, such activity correlates with the capacity of tested immunoglobulin to bind Gamma receptors exposed on THP-1 cell membrane. The stronger the activity, the higher the affinity.

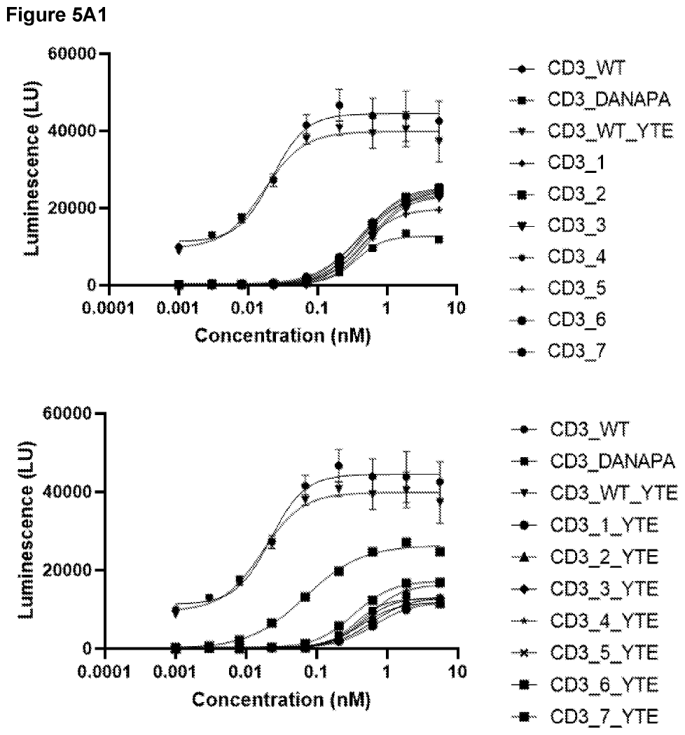

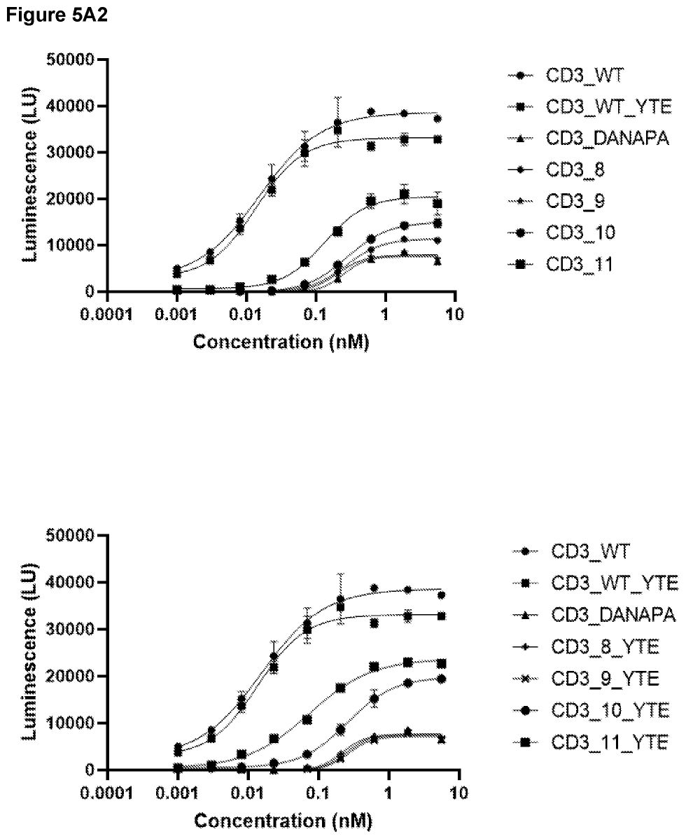

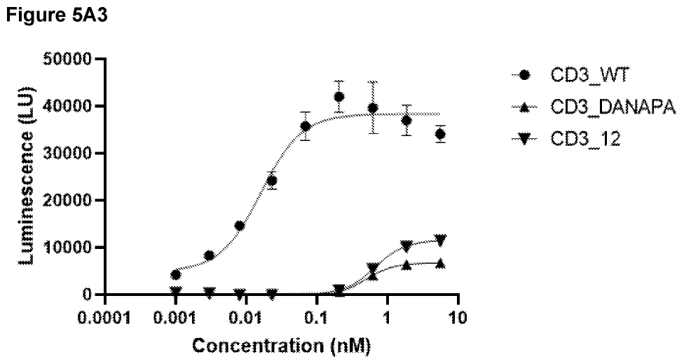

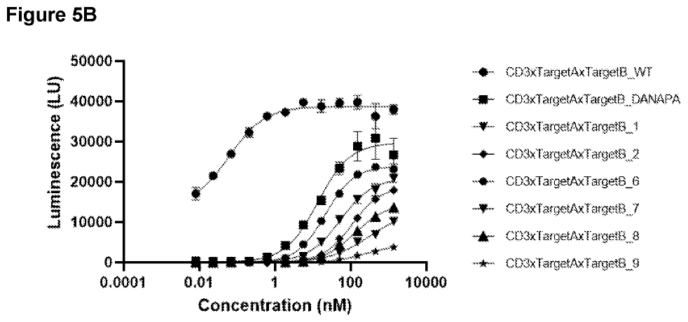

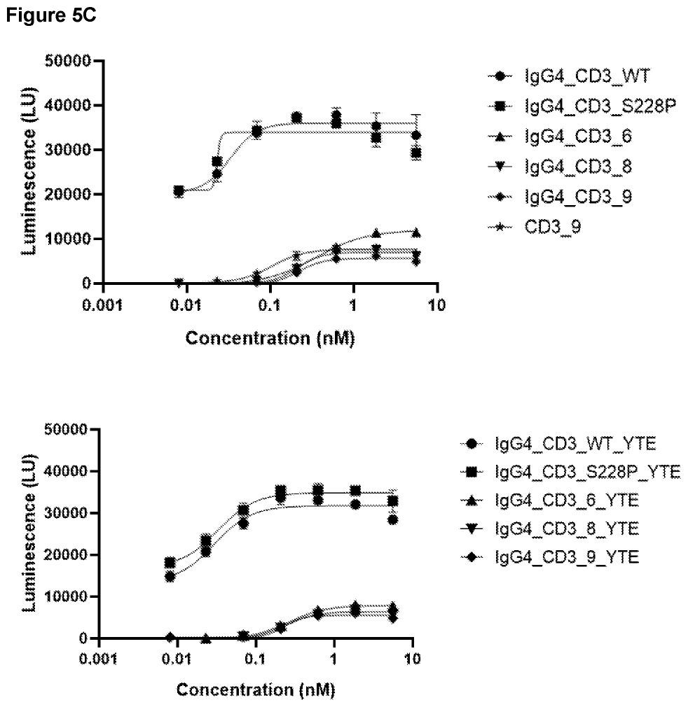

FIG. 5A1, FIG. 5A2 and FIG. 5A3 present results obtained using engineered anti-CD3 monospecific hIgG1 in separated assays (first assay: FIG. 5A1, second assay: FIG. 5A2, third assay: FIG. 5A3). In summary, parental (CD3_WT) and corresponding half-life extended variant (CD3_WT_YTE) show the greatest NFAT activity while all engineered immunoglobulins showed significantly dampened NFAT activation.

FIG. 5B presents results obtained using engineered anti-CD3xTargetAxTargetB trispecific hIgG1. In summary, parental (CD3xTargetAxTargetB_WT) shows the greatest NFAT activity while all engineered immunoglobulins showed significantly dampened NFAT activation.

FIG. 5C presents results obtained using engineered anti-CD3 monospecific hIgG4. In summary, parental (IgG4_WT or IgG4_CD3_S228P) and corresponding half-life extended variant (IgG4_CD3_WT_YTE or IgG4_CD3_S228P_YTE) show the greatest NFAT activity while all engineered immunoglobulins showed significantly dampened NFAT activation.

Those results taken together demonstrate that stabilization of IgG1 immunoglobulin by introduction of IgA top CH2 structural element results in strong decrease of interaction with Gamma receptors. In line with previous SPR measurements (vs FcγRI, FcγRII, and FcγRIII) presented Example 4, this cell-based assay confirms the surprising silencing effect of such stabilizing engineering. Interestingly, some variants exhibit measured silencing effect at least as strong as silencing effect observed when introduction of DANAPA mutation set, used as benchmark.

TABLE 8
Exemplary protein and nucleotide sequences

| | Internal | | |
|---|---|---|---|
| SEQ ID | reference | Name | Sequence |
| 1 | 2110 | Protein sequence NO 1 | SVKDRFTISRDDSKSTLYLQMNSLKTEDTAVYYCVRHGNFGNSYVSWFAYWGQGTLVTVSSASTK |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLMISRT |
| | | | PEVTCVVVDVSHEDPEVKFNWYVDGVEVHNAKTKPREEQYNSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 2 | | Nucleotide sequence of SEQ ID NO 1 | gaagtgcagctggtggaatctggcggcggactggtgcagcctggcggatctctgaagctgagctgtgccgccagcggcttca |
| | | | ccttcaacacctacgccatgaactgggtgcgccaggcctctggcaagggcctggaatggggggacggatcagaagcaagt |
| | | | acaacaattacgccacctactacgccgacagcgtgaaggaccggttcaccatcagccgggacgacagcaagagcaccctgt |
| | | | acctgcagatgaacagcctgaaaaccgaggacaccgccgtgtactactgcgtgcggcacggcaacttcggcaacagctatg |
| | | | tgtcttggtttgcctactggggccagggcaccctcgtgacagtgagctcagctagcaccaagggccccagcgtgttccccctg |
| | | | gcgcccagcagcaagagcaccagcggcggcacagccgccctgggctgcctggtgaaggactacttccccgagccagtgac |
| | | | cgtgtcctggaacagcggagccctgacctccggcgtgcacaccttccccgccgtgctgcagagcagcggcctgtacagcctg |
| | | | agcagcgtggtgaccgtgcccagcagcagcctgggcacccagacctacatctgcaacgtgaaccacaagcccagcaacac |
| | | | caaggtggacaagagagtggagcccaagagctgcgacaagacccacacctgccccccctgtcctgcccctgaactgctggg |
| | | | cggaccctccgtgttcctgttccccccaaagcccaaggacaccctgatgatcagccggacccccgaagtgacctgcgtggtg |
| | | | gtggacgtgtcccacgaggaccctgaagtgaagttcaattggtacgtggacggcgtggaagtgcacaacgccaagaccaag |
| | | | cccagagaggaacagtacaacagcacctaccgggtggtgtccgtgctgaccgtgctgcaccaggactggctgaacggcaaa |
| | | | gagtacaagtgcaaagtctccaacaaggccctgcctgcccccatcgagaaaaccatcagcaaggccaagggccagccccg |
| | | | cgagccccaggtgtacacactgccccccagccgggacgagctgaccaagaaccaggtgtccctgacctgcctggtcaaggg |
| | | | cttctaccccagcgatatcgccgtggaatgggagagcaacggccagcccgagaacaactacaagaccaccccccctgtgct |
| | | | ggacagcgacggctcattcttcctgtacagcaagctgaccgtggacaagtcccggtggcagcagggcaacgtgttcagctgc |
| | | | agcgtgatgcacgaggccctgcacaaccactacacccagaagtccctgagcctgagccccggcaaa |
| 3 | 2112 | Protein sequence NO 3 | QAVVTQEPSLTVSPGGTVTLTCRSSTGAVTTSNYANWVQQKPGQAPRGLIGGTNKRAPWTPARF |
| | | | SGSLLGDKAALTLSGAQPEDEAEYFCALWYSNLWVFGGGTKLTVLGQPKAAPSVTLFPPSSEELQA |
| | | | NKATLVCLISDFYPGAVTVAWKADSSPVKAGVETTTPSKQSNNKYAASSYLSLTPEQWKSHRSYSC |
| | | | QVTHEGSTVEKTVAPTECS |
| 4 | | Nucleotide sequence of SEQ ID NO 3 | caggctgtcgtgacccaggaacctagcctgaccgtgtctcctggcggaaccgtgaccctgacctgtagatctagcacaggcg |
| | | | ccgtgaccaccagcaactacgccaattgggtgcagcagaagcccggccaggctcctagaggactgatcggggcaccaac |
| | | | aagagagccccttggacccctgccagattcagcggctctctgctgggagataaggccgccctgacactgtctggcgcccagc |
| | | | ctgaggatgaggccgagtacttttgcgccctgtggtacagcaacctgtgggtgttcggcggaggcaccaagctgaccgtgct |
| | | | gggccagcctaaggccgctccctccgtgaccctgttcccccccagctccgaggaactgcaggccaacaaggccaccctggtg |
| | | | tgcctgatcagcgacttctaccctggcgccgtgaccgtggcctggaaggccgacagcagccccgtgaaggccggcgtggag |
| | | | acaaccacccccagcaagcagagcaacaacaagtacgccgccagcagctacctgagcctgacccccgagcagtggaaga |
| | | | gccacagaagctacagctgccaggtcacccacgagggcagcaccgtggagaaaaccgtggcccccaccgagtgcagc |
| 5 | 3447 | Proteinsequence NO 5 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLYITREP |
| | | | EVTCVVVDVSHEDPEVKFNWYVDGVEVHNAKTKPREEQYNSTYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 6 | | Nucleotide sequence of SEQ ID NO 5 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacacactgtacatcacccgcgagcctgaagtgacctgtgtggtggtggatgtgtcc |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 7 | 3448 | Protein sequence NO 7 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPRVFLFPPKPKDTLMISRT |
| | | | PEVTCVVTGLRDEDPEVKFNWYVDGVEVHNAKTKPREEQYCGCYSVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 8 | | Nucleotide sequence of SEQ ID NO 7 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgctgtcaccccaga |
| | | | gtgtttctgttccctccaaagcctaaggacaccctgatgatcagcagaacccctgaagtgacctgtgtcgtgaccggcctgag |
| | | | agatgaggaccccgaagtgaagttcaattggtacgtggacggcgtggaagtgcacaacgccaagaccaagcctagagagg |
| | | | aacagtactgcggctgctacagcgtggtgtctgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacatgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 9 | 3449 | Protein sequence NO 9 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPRVFLFPPKPKDTLYITRE |
| | | | PEVTCVVTGLRDEDPEVKFNWYVDGVEVHNAKTKPREEQYCGCYSVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 10 | | Nucleotide sequence of SEQ ID NO 9 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgctgtcaccccaga |
| | | | gtgtttctgttccctccaaagcctaaggacacactgtacatcacccgcgagcctgaagtgacctgtgtcgtgacaggactgag |
| | | | agatgaggaccccgaagtgaagttcaattggtacgtggacggcgtggaagtgcacaacgccaagaccaagcctagagagg |
| | | | aacagtactgcggctgctacagcgtggtgtctgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacatgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 11 | 3450 | Protein sequence NO 11 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLMISRT |
| | | | PEVTCVVVGLRHEDPEVKFNWYVDGVEVHNAKTKPREEQYCGCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 12 | | Nucleotide sequence of SEQ ID NO 11 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacaccctgatgatcagcagaacccctgaagtgacctgtgtggtcgtgggactgaga |
| | | | cacgaggaccccgaagtgaagttcaattggtacgtggacggcgtggaagtgcacaacgccaagaccaagcctagagagga |
| | | | acagtactgcggctgctacagagtggtgtctgtgctgacagtgctgcaccaggactggctgaacggcaaagagtacaagtgc |
| | | | aaggtgtccaacaaggccctgcctgctcctatcgagaaaaccatcagcaaggccaagggccagccaagagaaccccaggt |
| | | | ttacacactgcctccaagcagggacgagctgaccaagaatcaggtgtccctgacctgcctcgtgaagggcttctacccttccg |
| | | | atatcgccgtggaatgggagagcaatggccagcctgagaacaactacaagacaacccctcctgtgctggacagcgacggct |
| | | | cattcttcctacagcaagctgaccgtggacaagagcatggcagcagggcaacgtgttcagctgttctgtgatgcacga |
| | | | ggccctgcacaaccactacacccagaagtctctgtctctgagccccggcaag |
| 13 | 3451 | Protein sequence NO 13 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLYITREP |
| | | | EVTCVVVGLRHEDPEVKFNWYVDGVEVHNAKTKPREEQYCGCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 14 | | Nucleotide sequence of SEQ ID NO 13 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacacactgtacatcacccgcgagcctgaagtgacatgtgtggttgtgggactgaga |
| | | | cacgaggaccccgaagtgaagttcaattggtacgtggacggcgtggaagtgcacaacgccaagaccaagcctagagagga |
| | | | acagtactgcggctgctacagagtggtgtctgtgctgacagtgctgcaccaggactggctgaacggcaaagagtacaagtgc |
| | | | aaggtgtccaacaaggccctgcctgctcctatcgagaaaaccatcagcaaggccaagggccagccaagagaaccccaggt |
| | | | ttacacactgcctccaagcagggacgagctgaccaagaatcaggtgtccctgacctgcctcgtgaagggcttctacccttccg |
| | | | atatcgccgtggaatgggagagcaatggccagcctgagaacaactacaagacaacccctcctgtgctggacagcgacggct |
| | | | cattcttcctgtacagcaagctgaccgtggacaagagcagatggcagcagggcaacgtgttcagctgttctgtgatgcacga |
| | | | ggccctgcacaaccactacacccagaagtctctgtctctgagccccggcaag |
| 15 | 3452 | Protein sequence NO 15 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLMISRT |
| | | | PEVTCVVTGLRDEDPEVKFNWYVDGVEVHNAKTKPREEQYCGCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 16 | | Nucleotide sequence of SEQ ID NO 15 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacaccctgatgatcagcagaacccctgaagtgacctgtgtcgtgaccggcctgaga |
| | | | gatgaggaccccgaagtgaagttcaattggtacgtggacggcgtggaagtgcacaacgccaagaccaagcctagagagga |
| | | | acagtactgcggctgctacagagtggtgtctgtgctgacagtgctgcaccaggactggctgaacggcaaagagtacaagtgc |
| | | | aaggtgtccaacaaggccctgcctgctcctatcgagaaaaccatcagcaaggccaagggccagccaagagaaccccaggt |
| | | | ttacacactgcctccaagcagggacgagctgaccaagaatcaggtgtccctgacatgcctcgtgaagggcttctacccttccg |
| | | | atatcgccgtggaatgggagagcaatggccagcctgagaacaactacaagacaacccctcctgtgctggacagcgacggct |
| | | | cattcttcctgtacagcaagctgaccgtggacaagagcagatggcagcagggcaacgtgttcagctgttctgtgatgcacga |
| | | | ggccctgcacaaccactacacccagaagtctctgtctctgagccccggcaag |
| 17 | 3453 | Protein sequence NO 17 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLYITREP |
| | | | EVTCVVTGLRDEDPEVKFNWYVDGVEVHNAKTKPREEQYCGCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 18 | | Nucleotide sequence of SEQ ID NO 17 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacacactgtacatcacccgcgagcctgaagtgacctgtgtcgtgacaggactgaga |
| | | | gatgaggaccccgaagtgaagttcaattggtacgtggacggcgtggaagtgcacaacgccaagaccaagcctagagagga |
| | | | acagtactgcggctgctacagagtggtgtctgtgctgacagtgctgcaccaggactggctgaacggcaaagagtacaagtgc |
| | | | aaggtgtccaacaaggccctgcctgctcctatcgagaaaaccatcagcaaggccaagggccagccaagagaaccccaggt |
| | | | ttacacactgcctccaagcagggacgagctgaccaagaatcaggtgtccctgacatgcctcgtgaagggcttctacccttccg |
| | | | atatcgccgtggaatgggagagcaatggccagcctgagaacaactacaagacaacccctcctgtgctggacagcgacggct |
| | | | cattcttcctgtacagcaagctgaccgtggacaagagcagatggcagcagggcaacgtgttcagctgttctgtgatgcacga |
| | | | ggccctgcacaaccactacacccagaagtctctgtctctgagccccggcaag |
| 19 | 3454 | Protein sequence NO 19 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLMISRT |
| | | | PEVTCVVVDVSHEDPEVKFNWYVDGVEVHNAKTKPREEQYCGCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 20 | | Nucleotide sequence of SEQ ID NO 19 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacaccctgatgatcagcagaacccctgaagtgacctgcgtggtggtggatgtgtctc |
| | | | acgaggaccccgaagtgaagttcaattggtacgtggacggcgtggaagtgcacaacgccaagaccaagcctagagaggaa |
| | | | cagtactgcggctgctacagagtggtgtctgtgctgacagtgctgcaccaggactggctgaacggcaaagagtacaagtgca |
| | | | aggtgtccaacaaggccctgcctgctcctatcgagaaaaccatcagcaaggccaagggccagccaagagaaccccaggttt |
| | | | acacactgcctccaagcagggacgagctgaccaagaatcaggtgtccctgacctgcctcgtgaagggcttctacccttccga |
| | | | tatcgccgtggaatgggagagcaatggccagcctgagaacaactacaagacaacccctcctgtgctggacagcgacggctc |
| | | | attcttcctgtacagcaagctgaccgtggacaagagcagatggcagcagggcaacgtgttcagctgttctgtgatgcacgag |
| | | | gccctgcacaaccactacacccagaagtctctgtctctgagccccggcaag |
| 21 | 3455 | Protein sequence NO 21 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCHPSVFLFPPKPKDTLYITREP |
| | | | EVTCVVVDVSHEDPEVKFNWYVDGVEVHNAKTKPREEQYCGCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 22 | | Nucleotide sequence of SEQ ID NO 21 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgtcacccttccg |
| | | | tgtttctgttccctccaaagcctaaggacacactgtacatcacccgcgagcctgaagtgacctgtgtggtggtggatgtgtccc |
| | | | acgaggaccccgaagtgaagttcaattggtacgtggacggcgtggaagtgcacaacgccaagaccaagcctagagaggaa |
| | | | cagtactgcggctgctacagagtggtgtctgtgctgacagtgctgcaccaggactggctgaacggcaaagagtacaagtgca |
| | | | aggtgtccaacaaggccctgcctgctcctatcgagaaaaccatcagcaaggccaagggccagccaagagaaccccaggttt |
| | | | acacactgcctccaagcagggacgagctgaccaagaatcaggtgtccctgacctgcctcgtgaagggcttctacccttccga |
| | | | tatcgccgtggaatgggagagcaatggccagcctgagaacaactacaagacaacccctcctgtgctggacagcgacggctc |
| | | | attcttcctgtacagcaagctgaccgtggacaagagcagatggcagcagggcaacgtgttcagctgttctgtgatgcacgag |
| | | | gccctgcacaaccactacacccagaagtctctgtctctgagccccggcaag |
| 23 | 3456 | Protein sequence NO 23 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCGPSVFLFPPKPKDTLMISRT |
| | | | PEVTCVVVDVSHEDPEVKFNWYVDGVEVHNAKTKPREEQYCSCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 24 | | Nucleotide sequence of SEQ ID NO 23 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgcggccctagc |
| | | | gttttcctgtttcctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtattgcagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 25 | 3457 | Protein sequence NO 25 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCGPSVFLFPPKPKDTLYITREP |
| | | | EVTCVVVDVSHEDPEVKFNWYVDGVEVHNAKTKPREEQYCSCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 26 | | Nucleotide sequence of SEQ ID NO 25 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgcggccctagc |
| | | | gttttcctgtttcctccaaagcctaaggacaccctctacatcacccgcgagcctgaagtgacatgtgtggtggtggatgtgtcc |
| | | | cacgaggaccccgaagtgaagttcaattggtacgtggacggcgtggaagtgcacaacgccaagaccaagcctagagagga |
| | | | acagtattgcagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 27 | 3458 | Protein sequence NO 27 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCGPSVFLFPPKPKDTLMISRT |
| | | | PEVTCVVVDVSHEDPEVKFNWYVDGVEVHNAKTKPREEQYNSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 28 | | Nucleotide sequence of SEQ ID NO 27 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgcggccctagc |
| | | | gttttcctgtttcctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 29 | 3459 | Protein sequence NO 29 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCCGPSVFLFPPKPKDTLYITREP |
| | | | EVTCVVVDVSHEDPEVKFNWYVDGVEVHNAKTKPREEQYNSTYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 30 | | Nucleotide sequence of SEQ ID NO 29 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgttgcggccctagc |
| | | | gttttcctgtttcctccaaagcctaaggacaccctctacatcacccgcgagcctgaagtgacatgtgtggtggtggatgtgtcc |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 31 | 3460 | Protein sequence NO 31 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLMISRT |
| | | | PEVTCVVVDVSHEDPEVKFNWYVDGVEVHNAKTKPREEQYCSCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 32 | | Nucleotide sequence of SEQ ID NO 31 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtattgcagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 33 | 3461 | Protein sequence NO 33 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLYITREP |
| | | | EVTCVVVDVSHEDPEVKFNWYVDGVEVHNAKTKPREEQYCSCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 34 | | Nucleotide sequence of SEQ ID NO 33 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacacactgtacatcacccgcgagcctgaagtgacctgtgtggtggtggatgtgtcc |
| | | | cacgaggaccccgaagttcaattggtacgtggacggcgtggaagtgcacaacgccaagaccaagcctagagagga |
| | | | acagtattgcagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 35 | 3498 | Protein sequence NO 35 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLCGPSVFLFPPKPKDTLMISRT |
| | | | PEVTCVVVDVSHEDPEVKFNWYVDGVEVHNAKTKPREEQYNSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 36 | | Nucleotide sequence of SEQ ID NO 35 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgctctgcggccctagc |
| | | | gttttcctgtttcctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 37 | 3499 | Protein sequence NO 37 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCGGPSVFLFPPKPKDTLMISRT |
| | | | PEVTCVVVDVSHEDPEVKFNWYVDGVEVHNAKTKPREEQYNSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 38 | | Nucleotide sequence of SEQ ID NO 37 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgtggcggccctagc |
| | | | gttttcctgtttcctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 39 | 3500 | Protein sequence NO 39 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLCGPSVFLFPPKPKDTLYITREP |
| | | | EVTCVVVDVSHEDPEVKFNWYVDGVEVHNAKTKPREEQYNSTYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 40 | | Nucleotide sequence of SEQ ID NO 39 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgctgtgcggccctagc |
| | | | gttttcctgtttcctccaaagcctaaggacaccctctacatcacccgcgagcctgaagtgacatgtgtggtggtggatgtgtcc |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 41 | 3501 | Protein sequence NO 41 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCGGPSVFLFPPKPKDTLYITRE |
| | | | PEVTCVVVDVSHEDPEVKFNWYVDGVEVHNAKTKPREEQYNSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 42 | | Nucleotide sequence of SEQ ID NO 41 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgtggcggccctagc |
| | | | gttttcctgtttcctccaaagcctaaggacaccctctacatcacccgcgagcctgaagtgacatgtgtggtggtggatgtgtcc |
| | | | cacgaggaccccgaagtgaagttcaattggtacgtggacggcgtggaagtgcacaacgccaagaccaagcctagagagga |
| | | | acagtacaacagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 43 | 3502 | Protein sequence NO 43 | EVLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLMISRT |
| | | | PEVTCVVVDVSHEDPEVKFNWYVDGVEVHNAKTKPREEQYNSCYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 44 | | Nucleotide sequence of SEQ ID NO 43 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtataacagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 45 | 3503 | Protein sequence NO 45 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLMISRT |
| | | | PEVTCVVVDVSHEDPEVKFNWYVDGVEVHNAKTKPREEQYCSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 46 | | Nucleotide sequence of SEQ ID NO 45 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtattgcagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 47 | 3504 | Protein sequence NO 47 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLYITREP |
| | | | EVTCVVVDVSHEDPEVKFNWYVDGVEVHNAKTKPREEQYNSCYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 48 | | Nucleotide sequence of SEQ ID NO 47 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacacactgtacatcacccgcgagcctgaagtgacctgtgtggtggtggatgtgtcc |
| | | | cacgaggaccccgaagtgaagttcaattggtacgtggacggcgtggaagtgcacaacgccaagaccaagcctagagagga |
| | | | acagtataacagctgctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 49 | 3505 | Protein sequence NO 49 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLYITREP |
| | | | EVTCVVVDVSHEDPEVKFNWYVDGVEVHNAKTKPREEQYCSTYRVVSVLTVLHQDWLNGKEYKC |
| | | | KVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 50 | | Nucleotide sequence of SEQ ID NO 49 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagaactgctcggcggaccctcc |
| | | | gtgtttctgttccctccaaagcctaaggacacactgtacatcacccgcgagcctgaagtgacctgtgtggtggtggatgtgtcc |
| | | | cacgaggaccccgaagtgaagttcaattggtacgtggacggcgtggaagtgcacaacgccaagaccaagcctagagagga |
| | | | acagtattgcagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 51 | 3509 | Protein sequence NO 51 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCGGPSVFLFPPKPKDTLMISRT |
| | | | PEVTCVVVDVSHEDPEVKFNWYVDGVEVHNAKTKPREEQYCSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 52 | | Nucleotide sequence of SEQ ID NO 51 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgtggcggccctagc |
| | | | gttttcctgtttcctccaaagcctaaggacaccctgatgatcagcagaacccctgaagtgacctgcgtggtggtggatgtgtct |
| | | | cacgaggaccccgaagtgaagttcaattggtacgtggacggcgtggaagtgcacaacgccaagaccaagcctagagagga |
| | | | acagtattgcagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 53 | 3510 | Protein sequence NO 53 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELCGGPSVFLFPPKPKDTLYITRE |
| | | | PEVTCVVVDVSHEDPEVKFNWYVDGVEVHNAKTKPREEQYCSTYRVVSVLTVLHQDWLNGKEYK |
| | | | CKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 54 | | Nucleotide sequence of SEQ ID NO 53 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttccactggct |
| | | | cctagcagcaagtctacctctggtggaacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagctctagcctgggcacccagacctacatctgcaacgtgaaccacaagcctagcaacaccaaggtcga |
| | | | caagagagtggaacccaagagctgcgacaagacccacacctgtcctccatgtcctgctccagagctgtgtggcggccctagc |
| | | | gttttcctgtttcctccaaagcctaaggacaccctctacatcacccgcgagcctgaagtgacatgtgtggtggtggatgtgtcc |
| | | | cacgaggaccccgaagtgaagttcaattggtacgtggacggcgtggaagtgcacaacgccaagaccaagcctagagagga |
| | | | acagtattgcagcacctacagagtggtgtccgtgctgacagtgctgcaccaggactggctgaacggcaaagagtacaagtg |
| | | | caaggtgtccaacaaggccctgcctgctcctatcgagaaaaccatcagcaaggccaagggccagccaagagaaccccagg |
| | | | tttacacactgcctccaagcagggacgagctgaccaagaatcaggtgtccctgacctgcctcgtgaagggcttctacccttcc |
| | | | gatatcgccgtggaatgggagagcaatggccagcctgagaacaactacaagacaacccctcctgtgctggacagcgacgg |
| | | | ctcattcttcctgtacagcaagctgaccgtggacaagagcagatggcagcagggcaacgtgttcagctgttctgtgatgcacg |
| | | | aggccctgcacaaccactacacccagaagtctctgtctctgagccccggcaag |
| 55 | 3423 | Protein sequence NO 55 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLMISRT |
| | | | PEVTCVVVAVSHEDPEVKFNWYVDGVEVHNAKTKPREEQYASTYRVVSVLTVLHQDWLNGKEYK |
| | | | CKVSNKALAAPIEKTISKAKGQPREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEALHNHYTQKSLSLSPGK |
| 56 | | Nucleotide sequence of SEQ ID NO 55 | gaagtgcagctggtggaatctggcggcggactggtgcagcctggcggatctctgaagctgagctgtgccgccagcggcttca |
| | | | ccttcaacacctacgccatgaactgggtgcgccaggcctctggcaagggcctggaatgggtgggacggatcagaagcaagt |
| | | | acaacaattacgccacctactacgccgacagcgtgaaggaccggttcaccatcagccgggacgacagcaagagcaccctgt |
| | | | acctgcagatgaacagcctgaaaaccgaggacaccgccgtgtactactgcgtgcggcacggcaacttcggcaacagctatg |
| | | | tgtcttggtttgcctactggggccagggcaccctcgtgacagtgagctcagctagcaccaagggccccagcgtgttccccctg |
| | | | gcgccctctagcaagagcaccagcggaggaacagccgccctgggctgcctggtcaaggactactttcccgagcccgtgacc |
| | | | gtgtcctggaacagcggagcactgaccagcggcgtccacacctttccagccgtgctccagagcagcggcctgtactctctga |
| | | | gcagcgtggtgaccgtgcctagcagcagcctgggcacccagacctacatctgtaacgtgaaccacaagcccagcaacacca |
| | | | aggtggacaagagagtggaacccaagtcttgcgacaagacccacacctgccctccctgtccagcccctgaactgctgggag |
| | | | gccctagcgtgttcctgttccccccaaagcccaaggacaccctgatgatcagccggacccccgaagtgacctgtgtggtggtg |
| | | | gccgtgtctcacgaggaccctgaagtgaagtttaattggtacgtggacggcgtggaagtgcacaacgccaagaccaagccc |
| | | | agagaggaacagtacgccagcacctaccgggtggtgtccgtgctgacagtgctgcaccaggactggctgaacggcaaaga |
| | | | gtacaagtgcaaggtgtccaacaaggccctggccgctcccatcgagaaaaccatcagcaaggccaagggccagccccgcg |
| | | | aaccccaggtgtacacactgccccctagcagggacgagctgaccaagaaccaggtgtccctgacctgcctcgtgaagggctt |
| | | | ctacccctccgatatcgccgtggaatgggagagcaacggccagcccgagaacaactacaagaccaccccccctgtgctgga |
| | | | ctccgacggctcattcttcctgtacagcaagctgaccgtggacaagtcccggtggcagcagggcaacgtgttcagctgctccg |
| | | | tgatgcacgaggccctgcacaaccactacacccagaagtccctgagcctgagccccggcaaa |
| 57 | 3506 | Protein sequence NO 57 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPSSKSTSGGTAALGCLVKDYFPEPVTVSWNSGALTSGVHTFPAVLQSSGLYSLSSVVTV |
| | | | PSSSLGTQTYICNVNHKPSNTKVDKRVEPKSCDKTHTCPPCPAPELLGGPSVFLFPPKPKDTLYITREP |
| | | | EVTCVVVAVSHEDPEVKFNWYVDGVEVHNAKTKPREEQYASTYRVVSVLTVLHQDWLNGKEYKC |
| | | | KVSNKALAAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGK |
| 58 | | Nucleotide sequence of SEQ ID NO 57 | gaagtgcagctggtggaatctggcggcggactggtgcagcctggcggatctctgaagctgagctgtgccgccagcggcttca |
| | | | ccttcaacacctacgccatgaactgggtgcgccaggcctctggcaagggcctggaatggggggacggatcagaagcaagt |
| | | | acaacaattacgccacctactacgccgacagcgtgaaggaccggttcaccatcagccgggacgacagcaagagcaccctgt |
| | | | acctgcagatgaacagcctgaaaaccgaggacaccgccgtgtactactgcgtgcggcacggcaacttcggcaacagctatg |
| | | | tgtcttggtttgcctactggggccagggcaccctcgtgacagtgagctcagctagcaccaagggccccagcgtgttccccctg |
| | | | gcgccctctagcaagagcaccagcggaggaacagccgccctgggctgcctggtcaaggactactttcccgagcccgtgacc |
| | | | gtgtcctggaacagcggagcactgaccagcggcgtccacacctttccagccgtgctccagagcagcggcctgtactctctga |
| | | | gcagcgtggtgaccgtgcctagcagcagcctgggcacccagacctacatctgtaacgtgaaccacaagcccagcaacacca |
| | | | aggtggacaagagagtggaacccaagtcttgcgacaagacccacacctgccctccctgtccagcccctgaactgctgggag |
| | | | gccctagcgtgttcctgttccccccaaagcccaaggacaccctgtacatcacccgggagcccgaagtgacctgtgtggtggtg |
| | | | gccgtgtctcacgaggaccctgaagtgaagtttaattggtacgtggacggcgtggaagtgcacaacgccaagaccaagccc |
| | | | agagaggaacagtacgccagcacctaccgggtggtgtccgtgctgacagtgctgcaccaggactggctgaacggcaaaga |
| | | | gtacaagtgcaaggtgtccaacaaggccctggccgctcccatcgagaaaaccatcagcaaggccaagggccagccccgcg |
| | | | aaccccaggtgtacacactgccccctagcagggacgagctgaccaagaaccaggtgtccctgacctgcctcgtgaagggctt |
| | | | ctacccctccgatatcgccgtggaatgggagagcaacggccagcccgagaacaactacaagaccaccccccctgtgctgga |
| | | | ctccgacggctcattcttcctgtacagcaagctgaccgtggacaagtcccggtggcagcagggcaacgtgttcagctgctccg |
| | | | tgatgcacgaggccctgcacaaccactacacccagaagtccctgagcctgagccccggcaaa |
| 59 | 3511 | Protein sequence NO 59 | Protein_sequence_of_anti_TargetA_VH_binder_EVQLVESGGGLVQPGGSLKLSCAASGFTF |
| | | | NTYAMNWVRQASGKGLEWVGRIRSKYNNYATYYADSVKDRFTISRDDSKSTLYLQMNSLKTEDTA |
| | | | VYYCVRHGNFGNSYVSWFAYWGQGTLVTVSSGGGGSGGGGSGGGGSGGGGSQAVVTQEPSLT |
| | | | VSPGGTVTLTCRSSTGAVTTSNYANWVQQKPGQAPRGLIGGTNKRAPWTPARFSGSLLGDKAALT |
| | | | LSGAQPEDEAEYFCALWYSNLWVFGGGTKLTVLGGGGSDKTHTCPPCPAPELLGGPSVFLFPPKPK |
| | | | DTLMISRTPEVTCVVVDVSHEDPEVKFNWYVDGVEVHNAKTKPREEQYNSTYRVVSVLTVLHQD |
| | | | WLNGKEYKCKVSNKALPAPIEKTISKAKGQPREPQVCTLPPSREEMTKNQVSLSCAVKGFYPSDIAV |
| | | | EWESNGQPENNYKTTPPVLDSDGSFFLVSKLTVDKSRWQQGNVFSCSVMHEALHNHYTQKSLSLS |
| | | | PGK |
| 60 | | Nucleotide sequence of SEQ ID NO 59 | gaagttcagcttgttgaatctggcggcggactggtgcaacctggcggatctctgaaactgtcttgtgccgcctccggcttcacc |
| | | (Fc region) | ttcaatacctacgccatgaactgggttcgacaagcctccggcaaaggactggaatgggtcggacggatcagaagcaagtac |
| | | | aacaactacgccacctactacgccgactccgtgaaggacagattcaccatcagccgggacgactccaagagcaccctgtac |
| | | | ctccagatgaactccctgaaaaccgaggacacagccgtctattattgcgtgcggcacggcaacttcggcaacagctatgtgt |
| | | | cttggtttgcctactggggacagggaaccctcgtgaccgtttcttcaggcggcggtggtagtggcggtggtggtagcggaggc |
| | | | ggtggatcaggtggcggcggttctcaagctgtggtcacacaagagcccagcctgacagtttctcctggcggaaccgtgacac |
| | | | tgacctgtagatctagcaccggcgcagtgaccaccagcaattacgctaactgggtgcagcagaagcccggccaagctccta |
| | | | gaggactgatcggaggcacaaacaagagagccccttggacaccagccagattttctggctctctgctgggcgataaggccg |
| | | | ctcttacactgtctggcgcacagcctgaagatgaggccgagtacttttgcgccctgtggtacagcaacctgtgggtgttcggc |
| | | | ggaggaacaaagctgacagttcttggaggcggcggaagcgacaagacccacacatgtcctccatgtcctgctccagaactg |
| | | | ctcggcggaccctccgtgtttctgttccctccaaagccaaaggacaccctgatgatcagcagaacccctgaagtgacctgcgt |
| | | | ggtggtggatgtgtctcacgaggacccagaagtgaagttcaattggtacgtggacggcgtggaagtgcacaacgccaagac |
| | | | caagcctagagaggaacagtacaacagcacctacagagtggtgtccgtgctgacagtgctgcaccaggactggctgaacgg |
| | | | caaagagtacaagtgcaaggtgtccaacaaggccctgcctgctcctatcgagaaaaccatcagcaaggccaagggccagc |
| | | | ctagggaacctcaagtgtgcactctgcctccaagccgggaagagatgaccaagaatcaggtgtccctgagctgcgccgtga |
| | | | agggcttttacccttccgatatcgccgtggaatgggagagcaatggccagccagagaacaactacaagaccacacctcctgt |
| | | | gctggacagcgacggctcattcttcctggtgtctaagctgaccgtggacaagagcagatggcagcagggcaacgtgttcagc |
| | | | tgttctgtgatgcacgaggccctgcacaaccactacacacagaagtccctgtctctgagccccggcaaa |
| 61 | | Protein sequence NO 61 | EVQLVESGGGLVQPGGSLKLSCAASGFTFNTYAMNWVRQASGKGLEWVGRIRSKYNNYATYYAD |
| | | (Fc region) | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELLGGPSVFLFPPKPKDTLMISRTPEVTCVVVAVSHEDPEVKFNWYVDGVEV |
| | | | HNAKTKPREEQYASTYRVVSVLTVLHQDWLNGKEYKCKVSNKALAAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 62 | | Nucleotide sequence of SEQ ID NO 61 | gaagttcagcttgttgaatctggcggcggactggtgcaacctggcggatctctgaaactgtcttgtgccgcctccggcttcacc |
| | | (Fc region) | ttcaatacctacgccatgaactgggttcgacaagcctccggcaaaggactggaatgggtcggacggatcagaagcaagtac |
| | | | aacaactacgccacctactacgccgactccgtgaaggacagattcaccatcagccgggacgactccaagagcaccctgtac |
| | | | ctccagatgaactccctgaaaaccgaggacacagccgtctattattgcgtgcggcacggcaacttcggcaacagctatgtgt |
| | | | cttggtttgcctactggggacagggaaccctcgtgaccgtttcttcaggcggcggtggtagtggcggtggtggtagcggaggc |
| | | | ggtggatcaggtggcggcggttctcaagctgtggtcacacaagagcccagcctgacagtttctcctggcggaaccgtgacac |
| | | | tgacctgtagatctagcaccggcgcagtgaccaccagcaattacgctaactgggtgcagcagaagcccggccaagctccta |
| | | | gaggactgatcggaggcacaaacaagagagccccttggacaccagccagattttctggctctctgctgggcgataaggccg |
| | | | ctcttacactgtctggcgcacagcctgaagatgaggccgagtacttttgcgccctgtggtacagcaacctgtgggtgttcggc |
| | | | ggaggaacaaagctgacagttcttggaggcggcggaagcgacaagacccacacatgtcctccatgtcctgctccagaactg |
| | | | ctcggcggaccctccgtgtttctgttccctccaaagccaaaggacaccctgatgatcagcagaacccctgaagtgacctgtgt |
| | | | ggtggtggccgtgtctcacgaggacccagaagtgaagttcaattggtacgtggacggcgtggaagtgcacaacgccaagac |
| | | | caagcctagagaggaacagtacgccagcacctacagagtggtgtctgtgctgacagtgctgcaccaggactggctgaacgg |
| | | | caaagagtacaagtgcaaggtgtccaacaaggccctggccgctcctatcgagaaaaccatctctaaggccaagggccagcc |
| | | | tcgggaacctcaagtgtgcactcttccacctagccgggaagagatgaccaagaaccaggtgtcactgagctgcgccgtgaa |
| | | | gggcttctacccttctgatatcgccgtggaatgggagagcaacggccagccagagaacaactacaagaccacacctcctgtg |
| | | | ctggacagcgacggctcattcttcctggtgtctaagctgaccgtggacaagagcagatggcagcagggcaacgtgttcagct |
| | | | gttctgtgatgcacgaggccctgcacaaccactacacacagaagtccctgtctctgagccccggcaaa |
| 63 | 3513 | Protein sequence NO 63 | EVQLVESGGGLVQPGGSLKLSCAASGFTFNTYAMNWVRQASGKGLEWVGRIRSKYNNYATYYAD |
| | | (Fc region) | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELCCHPRVFLFPPKPKDTLMISRTPEVTCVVTGLRDEDPEVKFNWYVDGVEV |
| | | | HNAKTKPREEQYCGCYSVVSVLTVLHQDWLNGKEYKCKVSNKALPAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 64 | | Nucleotide sequence of SEQ ID NO 63 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgtgttgtcatccg |
| | | | cgtgtttttctgtttccgccgaaaccgaaagataccctgatgattagtcgtaccccggaagttacctgtgttgttacaggtctgc |
| | | | gtgatgaagacccggaagtgaaatttaattggtatgtggatggcgtggaagtgcataatgccaaaaccaaaccgcgcgaag |
| | | | aacagtattgtggctgctatagcgttgttagcgttctgaccgttctgcatcaggattggctgaatggcaaagaatataaatgca |
| | | | aagtgagcaataaagccctgccggcaccgattgaaaaaaccattagcaaagcaaaaggccagccgcgtgaaccgcaggtt |
| | | | tgtacactgccgccgagtcgtgaagaaatgaccaaaaatcaggttagcctgagctgcgccgtgaaaggtttttatccgagcg |
| | | | atattgccgtggaatgggaaagtaatggccagccggaaaataattataaaaccacaccgccggtgctggatagtgatggta |
| | | | gcttttttctggtgagcaaactgaccgtggataaaagccgttggcagcagggtaatgtgtttagctgtagcgttatgcatgaa |
| | | | gccctgcataatcattatacccagaaaagcctgagcctgagtccgggtaaa |
| 65 | 3514 | Protein sequence NO 65 | EVQLVESGGGLVQPGGSLKLSCAASGFTFNTYAMNWVRQASGKGLEWVGRIRSKYNNYATYYAD |
| | | (Fc region) | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELCCHPSVFLFPPKPKDTLMISRTPEVTCVVVGLRHEDPEVKFNWYVDGVEV |
| | | | HNAKTKPREEQYCGCYRVVSVLTVLHQDWLNGKEYKCKVSNKALPAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 66 | | Nucleotide sequence of SEQ ID NO 65 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgtgttgtcatccg |
| | | | agcgtttttctgtttccgccgaaaccgaaagataccctgatgattagtcgtaccccggaagttacctgcgttgttgttggtctgc |
| | | | gtcatgaagacccggaagtgaaatttaattggtatgtggatggcgtggaagtgcataatgccaaaaccaaaccgcgcgaag |
| | | | aacagtattgtggttgctatcgtgttgtgagcgttctgaccgttctgcatcaggattggctgaatggcaaagaatataaatgca |
| | | | aagtgagcaataaagccctgccggcaccgattgaaaaaaccattagcaaagcaaaaggccagccgcgtgaaccgcaggtt |
| | | | tgtacactgccgccgagtcgtgaagaaatgaccaaaaatcaggttagcctgagctgcgccgtgaaaggtttttatccgagcg |
| | | | atattgccgtggaatgggaaagtaatggccagccggaaaataattataaaaccacaccgccggtgctggatagtgatggta |
| | | | gcttttttctggtgagcaaactgaccgtggataaaagccgttggcagcagggtaatgtgtttagctgtagcgttatgcatgaa |
| | | | gccctgcataatcattatacccagaaaagcctgagcctgagtccgggtaaa |
| 67 | 3516 | Protein sequence NO 67 | EVQLVESGGGLVQPGGSLKLSCAASGFTFNTYAMNWVRQASGKGLEWVGRIRSKYNNYATYYAD |
| | | (Fc region) | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELCCGPSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 68 | | Nucleotide sequence of SEQ ID NO 67 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgtgttgtggtccg |
| | | | agtgtttttctgtttccgccgaaaccgaaagataccctgatgattagtcgtaccccggaagttacctgcgttgttgttgatgtga |
| | | | gccatgaagacccggaagtgaaatttaattggtatgtggatggcgtggaagtgcataatgccaaaaccaaaccgcgcgaag |
| | | | aacagtataatagcacctatcgtgttgtgagcgttctgaccgttctgcatcaggattggctgaatggcaaagaatataaatgc |
| | | | aaagtgagcaataaagccctgccggcaccgattgaaaaaaccattagcaaagcaaaaggccagccgcgtgaaccgcaggt |
| | | | ttgtacactgccgccgagtcgtgaagaaatgaccaaaaatcaggttagcctgagctgcgccgtgaaaggtttttatccgagc |
| | | | gatattgccgtggaatgggaaagtaatggccagccggaaaataattataaaaccacaccgccggtgctggatagtgatggt |
| | | | agcttttttctggtgagcaaactgaccgtggataaaagccgttggcagcagggtaatgtgtttagctgtagcgttatgcatga |
| | | | agccctgcataatcattatacccagaaaagcctgagcctgagtccgggtaaa |
| 69 | 3517 | Protein sequence NO 69 | EVQLVESGGGLVQPGGSLKLSCAASGFTFNTYAMNWVRQASGKGLEWVGRIRSKYNNYATYYAD |
| | | (Fc region) | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELLGGPSVFLFPPKPKDTLMISRTPEVTCVVVDVSHEDPEVKFNWYVDGVEV |
| | | | HNAKTKPREEQYCSCYRVVSVLTVLHQDWLNGKEYKCKVSNKALPAPIEKTISKAKGQPREPQVCTL |
| | | | PPSREEMTKNQVSLSCAVKGFYPSDIAVEWESNGQPENNYKTTPPVLDSDGSFFLVSKLTVDKSRW |
| | | | QQGNVFSCSVMHEALHNHYTQKSLSLSPGK |
| 70 | | Nucleotide sequence of SEQ ID NO 69 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgctgggtggtcc |
| | | | gagtgtttttctgtttccgccgaaaccgaaagataccctgatgattagtcgtaccccggaagttacctgcgttgttgttgatgtg |
| | | | agccatgaagacccggaagtgaaatttaattggtatgtggatggcgtggaagtgcataatgccaaaaccaaaccgcgcgaa |
| | | | gaacagtattgtagctgctatcgtgttgtgagcgttctgaccgttctgcatcaggattggctgaatggcaaagaatataaatgc |
| | | | aaagtgagcaataaagccctgccggcaccgattgaaaaaaccattagcaaagcaaaaggccagccgcgtgaaccgcaggt |
| | | | ttgtacactgccgccgagtcgtgaagaaatgaccaaaaatcaggttagcctgagctgcgccgtgaaaggtttttatccgagc |
| | | | gatattgccgtggaatgggaaagtaatggccagccggaaaataattataaaaccacaccgccggtgctggatagtgatggt |
| | | | agcttttttctggtgagcaaactgaccgtggataaaagccgttggcagcagggtaatgtgtttagctgtagcgttatgcatga |
| | | | agccctgcataatcattatacccagaaaagcctgagcctgagtccgggtaaa |
| 71 | 3518 | Protein sequence NO 71 (Fc region) | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELLCGPSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 72 | | Nucleotide sequence of SEQ ID NO 71 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgctgtgtggtccg |
| | | | agtgtttttctgtttccgccgaaaccgaaagataccctgatgattagtcgtaccccggaagttacctgcgttgttgttgatgtga |
| | | | gccatgaagacccggaagtgaaatttaattggtatgtggatggcgtggaagtgcataatgccaaaaccaaaccgcgcgaag |
| | | | aacagtataatagcacctatcgtgttgtgagcgttctgaccgttctgcatcaggattggctgaatggcaaagaatataaatgc |
| | | | aaagtgagcaataaagccctgccggcaccgattgaaaaaaccattagcaaagcaaaaggccagccgcgtgaaccgcaggt |
| | | | ttgtacactgccgccgagtcgtgaagaaatgaccaaaaatcaggttagcctgagctgcgccgtgaaaggtttttatccgagc |
| | | | gatattgccgtggaatgggaaagtaatggccagccggaaaataattataaaaccacaccgccggtgctggatagtgatggt |
| | | | agcttttttctggtgagcaaactgaccgtggataaaagccgttggcagcagggtaatgtgtttagctgtagcgttatgcatga |
| | | | agccctgcataatcattatacccagaaaagcctgagcctgagtccgggtaaa |
| 73 | 3519 | Protein sequence NO 73 (Fc region) | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSGGGG |
| | | | SGGGGSGGGGSGGGGSQAVVTQEPSLTVSPGGTVTLTCRSSTGAVTTSNYANWVQQKPGQAPR |
| | | | GLIGGTNKRAPWTPARFSGSLLGDKAALTLSGAQPEDEAEYFCALWYSNLWVFGGGTKLTVLGGG |
| | | | GSDKTHTCPPCPAPELCGGPSVFLFPPKPKDTLMISRTPEVTCVVVDVSHEDPEVKFNWYVDGVEV |
| | | | HNAKTKPREEQYNSTYRVVSVLTVLHQDWLNGKEYKCKVSNKALPAPIEKTISKAKGQPREPQVCT |
| | | | LPPSREEMTKNQVSLSCAVKGFYPSDIAVEWESNGQPENNYKTTPPVLDSDGSFFLVSKLTVDKSR |
| | | | WQQGNVFSCSVMHEALHNHYTQKSLSLSPGK |
| 74 | | Nucleotide sequence of SEQ ID NO 73 | gaagttcagctggttgaaagtggtggtggtctggttcagccgggtggtagtctgaaactgagttgtgcagcaagcggctttac |
| | | (Fc region) | ctttaatacctatgccatgaattgggtgcgtcaggcaagtggtaaaggtctggaatgggttggtcgtattcgcagcaaatata |
| | | | ataattatgccacctattatgccgatagcgtgaaagatcgctttaccattagccgtgatgatagcaaaagcaccctgtatttac |
| | | | agatgaatagcctgaaaaccgaagataccgccgtgtattattgcgtgcgtcatggcaattttggcaatagctatgtgagctgg |
| | | | tttgcctattggggtcagggtacactggttacagttagcagtggtggtggtggttcaggtggtggtggtagtggtggtggtggt |
| | | | tcaggtggtggtggtagtcaggcagttgttacacaggaaccgagtctgacagttagtccgggtggtacagttaccctgacctg |
| | | | tcgtagtagtacaggtgcagttaccaccagcaattatgccaattgggttcagcagaaaccgggtcaggcaccgcgtggtctg |
| | | | attggtggtacaaataaacgtgcaccgtggacaccggcacgttttagtggtagtctgctgggtgataaagcagcactgacac |
| | | | tgagtggtgcacagccggaagatgaagccgaatatttttgtgccctgtggtatagcaatctgtgggtttttggtggtggcacca |
| | | | aactgacagttctgggtggtggtggtagcgataaaacccatacatgtccgccgtgtccggcaccggaactgtgtggtggtccg |
| | | | agtgtttttctgtttccgccgaaaccgaaagataccctgatgattagtcgtaccccggaagttacctgcgttgttgttgatgtga |
| | | | gccatgaagacccggaagtgaaatttaattggtatgtggatggcgtggaagtgcataatgccaaaaccaaaccgcgcgaag |
| | | | aacagtataatagcacctatcgtgttgtgagcgttctgaccgttctgcatcaggattggctgaatggcaaagaatataaatgc |
| | | | aaagtgagcaataaagccctgccggcaccgattgaaaaaaccattagcaaagcaaaaggccagccgcgtgaaccgcaggt |
| | | | ttgtacactgccgccgagtcgtgaagaaatgaccaaaaatcaggttagcctgagctgcgccgtgaaaggtttttatccgagc |
| | | | gatattgccgtggaatgggaaagtaatggccagccggaaaataattataaaaccacaccgccggtgctggatagtgatggt |
| | | | agcttttttctggtgagcaaactgaccgtggataaaagccgttggcagcagggtaatgtgtttagctgtagcgttatgcatga |
| | | | agccctgcataatcattatacccagaaaagcctgagcctgagtccgggtaaa |
| 75 | 3520 | Protein sequence NO 75 (Fc region) | DKTHTCPPCPAPELLGGPSVFLFPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 76 | | Nucleotide sequence of SEQ ID NO 75 | gataagacacacacatgtcctccatgtcctgctccagagctgctcggcggaccttctgttttcctgtttccacctaagccaaag |
| | | (Fc region) | gacaccctgatgatcagcagaacccctgaagtgacctgcgtggtggtggatgtgtctcacgaggaccccgaagtgaagttca |
| | | | attggtacgtggacggcgtggaagtgcacaacgccaagaccaagcctagagaggaacagtacaacagcacctacagagtg |
| | | | gtgtccgtgctgaccgtgctgcaccaggattggctgaacggcaaagagtacaagtgcaaggtgtccaacaaggccctgcctg |
| | | | ctcctatcgagaaaaccatcagcaaggccaagggccagccaagagaaccccaggtttacaccctgcctccatgccgggaag |
| | | | agatgaccaagaatcaggtgtccctgtggtgcctggtcaagggcttctacccttccgatatcgccgtggaatgggagagcaa |
| | | | tggccagcctgagaacaactacaagaccacacctcctgtgctggacagcgacggctcattcttcctgtacagcaagctgaca |
| | | | gtggacaagagcagatggcagcagggcaacgtgttcagctgttctgtgatgcacgaggccctgcacaaccactacacccag |
| | | | aagtctctgtctctgagccccggcaaa |
| 77 | | Protein sequence NO 77 (Fc region) | DKTHTCPPCPAPELLGGPSVFLFPPKPKDTLMISRTPEVTCVVVAVSHEDPEVKFNWYVDGVEVHN |
| | | | AKTKPREEQYASTYRVVSVLTVLHQDWLNGKEYKCKVSNKALAAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 78 | | Nucleotide sequence of SEQ ID NO 77 | gataagacacacacatgtcctccatgtcctgctccagagctgctcggcggaccttctgttttcctgtttccacctaagccaaag |
| | | (Fc region) | gacaccctgatgatcagcagaacccctgaagtgacctgtgtggtggtggccgtgtctcacgaagatcccgaagtgaagttca |
| | | | attggtacgtggacggcgtggaagtgcacaacgccaagaccaagcctagagaggaacagtacgccagcacctatagagtg |
| | | | gtgtccgtgctgacagtgctgcaccaggactggctgaacggcaaagagtacaagtgcaaggtgtccaacaaggccctggcc |
| | | | gctcctatcgagaaaaccatctctaaggccaagggccagcctcgggaaccacaggtttacaccctgcctccatgccgggaag |
| | | | agatgaccaagaatcaggtgtccctgtggtgcctggtcaagggcttctacccttccgatatcgccgtggaatgggagagcaa |
| | | | tggccagcctgagaacaactacaagaccacacctcctgtgctggacagcgacggctcattcttcctgtacagcaagctgacc |
| | | | gtggacaagagcagatggcagcagggcaacgtgttcagctgttctgtgatgcacgaggccctgcacaaccactacacccag |
| | | | aagtctctgtctctgagccccggcaaa |
| 79 | 3522 | Protein sequence NO 79 | DKTHTCPPCPAPELCCHPRVFLFPPKPKDTLMISRTPEVTCVVTGLRDEDPEVKFNWYVDGVEVHN |
| | | (Fc region) | AKTKPREEQYCGCYSVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 80 | | Nucleotide sequence of SEQ ID NO 79 | gataaaacacatacatgtccgccgtgtccggcaccggaactgtgttgtcatccgcgtgtttttctgtttccgccgaaaccgaaa |
| | | (Fc region) | gataccctgatgattagtcgtaccccggaagttacctgtgttgttacaggtctgcgtgatgaagacccggaagtgaaatttaa |
| | | | ttggtatgtggatggcgtggaagtgcataatgccaaaaccaaaccgcgcgaagaacagtattgtggctgctatagcgttgtt |
| | | | agcgttctgaccgttctgcatcaggattggctgaatggcaaagaatataaatgcaaagtgagcaataaagccctgccggcac |
| | | | cgattgaaaaaaccattagcaaagcaaaaggccagccgcgtgaaccgcaggtttatacactgccgccgtgtcgtgaagaaa |
| | | | tgaccaaaaatcaggttagcctgtggtgcctggtgaaaggtttttatccgagcgatattgccgtggaatgggaaagtaatggc |
| | | | cagccggaaaataattataaaaccacaccgccggtgctggatagtgatggtagcttttttctgtatagcaaactgaccgtgga |
| | | | taaaagccgttggcagcagggtaatgtgtttagctgtagcgttatgcatgaagccctgcataatcattatacccagaaaagcc |
| | | | tgagcctgagtccgggtaaa |
| 81 | 3523 | Protein sequence NO 81 | DKTHTCPPCPAPELCCHPSVFLFPPKPKDTLMISRTPEVTCVVVGLRHEDPEVKFNWYVDGVEVHN |
| | | (Fc region) | AKTKPREEQYCGCYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 82 | | Nucleotide sequence of SEQ ID NO 81 | gataaaacacatacatgtccgccgtgtccggcaccggaactgtgttgtcatccgagcgtttttctgtttccgccgaaaccgaa |
| | | (Fc region) | agataccctgatgattagtcgtaccccggaagttacctgcgttgttgttggtctgcgtcatgaagacccggaagtgaaatttaa |
| | | | ttggtatgtggatggcgtggaagtgcataatgccaaaaccaaaccgcgcgaagaacagtattgtggttgctatcgtgttgtga |
| | | | gcgttctgaccgttctgcatcaggattggctgaatggcaaagaatataaatgcaaagtgagcaataaagccctgccggcacc |
| | | | gattgaaaaaaccattagcaaagcaaaaggccagccgcgtgaaccgcaggtttatacactgccgccgtgtcgtgaagaaat |
| | | | gaccaaaaatcaggttagcctgtggtgcctggtgaaaggtttttatccgagcgatattgccgtggaatgggaaagtaatggc |
| | | | cagccggaaaataattataaaaccacaccgccggtgctggatagtgatggtagcttttttctgtatagcaaactgaccgtgga |
| | | | taaaagccgttggcagcagggtaatgtgtttagctgtagcgttatgcatgaagccctgcataatcattatacccagaaaagcc |
| | | | tgagcctgagtccgggtaaa |
| 83 | 3525 | Protein sequence NO 83 | DKTHTCPPCPAPELCCGPSVFLFPPKPKDTLMISRTPEVTCVVVDVSHEDPEVKFNWYVDGVEVHN |
| | | (Fc region) | AKTKPREEQYNSTYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 84 | | Nucleotide sequence of SEQ ID NO 83 | gataaaacacatacatgtccgccgtgtccggcaccggaactgtgttgtggtccgagtgtttttctgtttccgccgaaaccgaaa |
| | | (Fc region) | gataccctgatgattagtcgtaccccggaagttacctgcgttgttgttgatgtgagccatgaagacccggaagtgaaatttaa |
| | | | ttggtatgtggatggcgtggaagtgcataatgccaaaaccaaaccgcgcgaagaacagtataatagcacctatcgtgttgtg |
| | | | agcgttctgaccgttctgcatcaggattggctgaatggcaaagaatataaatgcaaagtgagcaataaagccctgccggcac |
| | | | cgattgaaaaaaccattagcaaagcaaaaggccagccgcgtgaaccgcaggtttatacactgccgccgtgtcgtgaagaaa |
| | | | tgaccaaaaatcaggttagcctgtggtgcctggtgaaaggtttttatccgagcgatattgccgtggaatgggaaagtaatggc |
| | | | cagccggaaaataattataaaaccacaccgccggtgctggatagtgatggtagcttttttctgtatagcaaactgaccgtgga |
| | | | taaaagccgttggcagcagggtaatgtgtttagctgtagcgttatgcatgaagccctgcataatcattatacccagaaaagcc |
| | | | tgagcctgagtccgggtaaa |
| 85 | 3526 | Protein sequence NO 85 | DKTHTCPPCPAPELLGGPSVFLFPPKPKDTLMISRTPEVTCVVVDVSHEDPEVKFNWYVDGVEVHN |
| | | (Fc region) | AKTKPREEQYCSCYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 86 | | Nucleotide sequence of SEQ ID NO 85 | gataaaacacatacatgtccgccgtgtccggcaccggaactgctgggtggtccgagtgtttttctgtttccgccgaaaccgaa |
| | | (Fc region) | agataccctgatgattagtcgtaccccggaagttacctgcgttgttgttgatgtgagccatgaagacccggaagtgaaattta |
| | | | attggtatgtggatggcgtggaagtgcataatgccaaaaccaaaccgcgcgaagaacagtattgtagctgctatcgtgttgt |
| | | | gagcgttctgaccgttctgcatcaggattggctgaatggcaaagaatataaatgcaaagtgagcaataaagccctgccggc |
| | | | accgattgaaaaaaccattagcaaagcaaaaggccagccgcgtgaaccgcaggtttatacactgccgccgtgtcgtgaaga |
| | | | aatgaccaaaaatcaggttagcctgtggtgcctggtgaaaggtttttatccgagcgatattgccgtggaatgggaaagtaat |
| | | | ggccagccggaaaataattataaaaccacaccgccggtgctggatagtgatggtagcttttttctgtatagcaaactgaccgt |
| | | | ggataaaagccgttggcagcagggtaatgtgtttagctgtagcgttatgcatgaagccctgcataatcattatacccagaaa |
| | | | agcctgagcctgagtccgggtaaa |
| 87 | 3527 | Protein sequence NO 87 | DKTHTCPPCPAPELLCGPSVFLFPPKPKDTLMISRTPEVTCVVVDVSHEDPEVKFNWYVDGVEVHN |
| | | (Fc region) | AKTKPREEQYNSTYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 88 | | Nucleotide sequence of SEQ ID NO 87 | gataaaacacatacatgtccgccgtgtccggcaccggaactgctgtgtggtccgagtgtttttctgtttccgccgaaaccgaa |
| | | (Fc region) | agataccctgatgattagtcgtaccccggaagttacctgcgttgttgttgatgtgagccatgaagacccggaagtgaaattta |
| | | | attggtatgtggatggcgtggaagtgcataatgccaaaaccaaaccgcgcgaagaacagtataatagcacctatcgtgttgt |
| | | | gagcgttctgaccgttctgcatcaggattggctgaatggcaaagaatataaatgcaaagtgagcaataaagccctgccggc |
| | | | accgattgaaaaaaccattagcaaagcaaaaggccagccgcgtgaaccgcaggtttatacactgccgccgtgtcgtgaaga |
| | | | aatgaccaaaaatcaggttagcctgtggtgcctggtgaaaggtttttatccgagcgatattgccgtggaatgggaaagtaat |
| | | | ggccagccggaaaataattataaaaccacaccgccggtgctggatagtgatggtagcttttttctgtatagcaaactgaccgt |
| | | | ggataaaagccgttggcagcagggtaatgtgtttagctgtagcgttatgcatgaagccctgcataatcattatacccagaaa |
| | | | agcctgagcctgagtccgggtaaa |
| 89 | 3528 | Protein sequence NO 89 (Fc region) | DKTHTCPPCPAPELCGGPSVFLFPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIEKTISKAKGQPREPQVYTLPP |
| | | | CREEMTKNQVSLWCLVKGFYPSDIAVEWESNGQPENNYKTTPPVLDSDGSFFLYSKLTVDKSRWQ |
| | | | QGNVFSCSVMHEALHNHYTQKSLSLSPGK |
| 90 | | Nucleotide sequence of SEQ ID NO 89 | gataaaacacatacatgtccgccgtgtccggcaccggaactgtgtggtggtccgagtgtttttctgtttccgccgaaaccgaa |
| | | (Fc region) | agataccctgatgattagtcgtaccccggaagttacctgcgttgttgttgatgtgagccatgaagacccggaagtgaaattta |
| | | | attggtatgtggatggcgtggaagtgcataatgccaaaaccaaaccgcgcgaagaacagtataatagcacctatcgtgttgt |
| | | | gagcgttctgaccgttctgcatcaggattggctgaatggcaaagaatataaatgcaaagtgagcaataaagccctgccggc |
| | | | accgattgaaaaaaccattagcaaagcaaaaggccagccgcgtgaaccgcaggtttatacactgccgccgtgtcgtgaaga |
| | | | aatgaccaaaaatcaggttagcctgtggtgcctggtgaaaggtttttatccgagcgatattgccgtggaatgggaaagtaat |
| | | | ggccagccggaaaataattataaaaccacaccgccggtgctggatagtgatggtagcttttttctgtatagcaaactgaccgt |
| | | | ggataaaagccgttggcagcagggtaatgtgtttagctgtagcgttatgcatgaagccctgcataatcattatacccagaaa |
| | | | agcctgagcctgagtccgggtaaa |
| 91 | 3529 | Protein sequence NO 91 | RTVAAPSVFIFPPSDEQLKSGTASVVCLLNNFYPREAKVQWKVDNALQSGNSQESVTEQDSKDSTY |
| | | (Fc region) | SLSSTLTLSKADYEKHKVYACEVTHQGLSSPVTKSFNRGEC |
| 92 | | Nucleotide sequence of SEQ ID NO 91 | agaacagtggccgctccgagcgtgttcatctttccaccaagcgacgagcagctgaaaagcggcacagcctctgtcgtgtgcc |
| | | (Fc region) | tgctgaacaacttctaccccagagaagccaaggtgcagtggaaggtggacaatgccctccagtccggcaatagccaagaga |
| | | | gcgtgaccgagcaggacagcaaggatagcacatacagcctgagcagcacactgaccctgagcaaggccgactacgagaa |
| | | | gcacaaagtgtacgcctgcgaagtgacacaccagggcctgtctagccctgtgaccaagagcttcaacagaggcgagtgc |
| 93 | 3548 | Protein sequence NO 93 | DKTHTCPPCPAPELCCHPRVFLFPPKPKDTLMISRTPEVTCVVTGLRDEDPEVKFNWYVDGVEVHN |
| | | | AKTKPREEQYCGCYSVVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLTVDKSRWQ |
| | | | QGNVFSCSVMHEALHNHYTQKSLSLSPGK |
| 94 | | Nucleotide sequence of SEQ ID NO 93 | gataagacccacacctgtcctccatgtcctgctccagagctgtgctgtcaccccagagtgtttctgttccctccaaagcctaag |
| | | | gacaccctgatgatcagcagaacccctgaagtgacctgtgtcgtgaccggcctgagagatgaggaccccgaagtgaagttc |
| | | | aattggtacgtggacggcgtggaagtgcacaacgccaagaccaagcctagagaggaacagtactgcggctgctacagcgt |
| | | | ggtgtctgtgctgacagtgctgcaccaggactggctgaacggcaaagagtacaagtgcaaggtgtccaacaaggccctgcc |
| | | | tgctcctatcgagaaaaccatcagcaaggccaagggccagccaagagaaccccaggtttacaccctgcctccaagccggga |
| | | | agagatgaccaagaatcaggtgtccctgacctgcctggtcaagggcttctacccttccgatatcgccgtggaatgggagagc |
| | | | aatggccagcctgagaacaactacaagaccacacctcctgtgctggacagcgacggctcattcttcctgtacagcaagctga |
| | | | ccgtggacaagagcagatggcagcagggcaacgtgttcagctgttctgtgatgcacgaggccctgcacaaccactacaccc |
| | | | agaagtctctgtctctgagccccggcaaa |
| 95 | 3551 | Protein sequence NO 95 | DKTHTCPPCPAPELCCGPSVFLFPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLTVDKSRWQ |
| | | | QGNVFSCSVMHEALHNHYTQKSLSLSPGK |
| 96 | | Nucleotide sequence of SEQ ID NO 95 | gataagacccacacctgtcctccatgtcctgctccagagctgtgttgcggcccctccgttttcctgtttccacctaagcctaagg |
| | | | acaccctgatgatcagcagaacccctgaagtgacctgcgtggtggtggatgtgtctcacgaggaccccgaagtgaagttcaa |
| | | | ttggtacgtggacggcgtggaagtgcacaacgccaagaccaagcctagagaggaacagtacaacagcacctacagagtgg |
| | | | tgtccgtgctgaccgtgctgcaccaggattggctgaacggcaaagagtacaagtgcaaggtgtccaacaaggccctgcctgc |
| | | | tcctatcgagaaaaccatcagcaaggccaagggccagccaagagaaccccaggtttacaccctgcctccaagccgggaag |
| | | | agatgaccaagaatcaggtgtccctgacctgcctggtcaagggcttctacccttccgatatcgccgtggaatgggagagcaa |
| | | | tggccagcctgagaacaactacaagaccacacctcctgtgctggacagcgacggctcattcttcctgtacagcaagctgaca |
| | | | gtggacaagagcagatggcagcagggcaacgtgttcagctgttctgtgatgcacgaggccctgcacaaccactacacccag |
| | | | aagtctctgtctctgagccccggcaaa |
| 97 | 3553 | Protein sequence NO 97 | DKTHTCPPCPAPELLCGPSVFLFPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLTVDKSRWQ |
| | | | QGNVFSCSVMHEALHNHYTQKSLSLSPGK |
| 98 | | Nucleotide sequence of SEQ ID NO 97 | gataagacccacacctgtcctccatgtcctgctccagaactgctgtgcggcccctccgttttcctgtttccacctaagcctaagg |
| | | | acaccctgatgatcagcagaacccctgaagtgacctgcgtggtggtggatgtgtctcacgaggaccccgaagtgaagttcaa |
| | | | ttggtacgtggacggcgtggaagtgcacaacgccaagaccaagcctagagaggaacagtacaacagcacctacagagtgg |
| | | | tgtccgtgctgaccgtgctgcaccaggattggctgaacggcaaagagtacaagtgcaaggtgtccaacaaggccctgcctgc |
| | | | tcctatcgagaaaaccatcagcaaggccaagggccagccaagagaaccccaggtttacaccctgcctccaagccgggaag |
| | | | agatgaccagaatcaggtgtccctgacctgcctggtcaagggcttctacccttccgatatcgccgtggaatgggagagcaa |
| | | | tggccagcctgagaacaactacaagaccacacctcctgtgctggacagcgacggctcattcttcctgtacagcaagctgaca |
| | | | gtggacaagagcagatggcagcagggcaacgtgttcagctgttctgtgatgcacgaggccctgcacaaccactacacccag |
| | | | aagtctctgtctctgagccccggcaaa |
| 99 | 3554 | Protein sequence NO 99 | DKTHTCPPCPAPELCGGPSVFLFPPKPKDTLMISRTPEVTCVVVDVSHEDPEVKFNWYVDGVEVHN |
| | | | AKTKPREEQYNSTYRVVSVLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDGSFFLYSKLTVDKSRWQ |
| | | | QGNVFSCSVMHEALHNHYTQKSLSLSPGK |
| 100 | | Nucleotide sequence of SEQ ID NO 99 | gataagacccacacctgtcctccatgtcctgctccagaactgtgtggcggccctagcgttttcctgtttcctccaaagcctaag |
| | | | gacaccctgatgatcagcagaacccctgaagtgacctgcgtggtggtggatgtgtctcacgaggaccccgaagtgaagttca |
| | | | attggtacgtggacggcgtggaagtgcacaacgccaagaccaagcctagagaggaacagtacaacagcacctacagagtg |
| | | | gtgtccgtgctgaccgtgctgcaccaggattggctgaacggcaaagagtacaagtgcaaggtgtccaacaaggccctgcctg |
| | | | ctcctatcgagaaaaccatcagcaaggccaagggccagccaagagaaccccaggtttacaccctgcctccaagccgggaa |
| | | | gagatgaccaagaatcaggtgtccctgacctgcctggtcaagggcttctacccttccgatatcgccgtggaatgggagagca |
| | | | atggccagcctgagaacaactacaagaccacacctcctgtgctggacagcgacggctcattcttcctgtacagcaagctgac |
| | | | agtggacaagagcagatggcagcagggcaacgtgttcagctgttctgtgatgcacgaggccctgcacaaccactacaccca |
| | | | gaagtctctgtctctgagccccggcaaa |
| 101 | 3557 | Protein sequence NO 101 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLMISRTPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 102 | | Nucleotide sequence of SEQ ID NO 101 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 103 | 3558 | Protein sequence NO 103 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPPCPAPEFLGGPSVFLFPPKPKDTLMISRTPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 104 | | Nucleotide sequence of SEQ ID NO 103 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctccatgtcctgctccagaatttctcggcggacccagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 105 | 3559 | Protein sequence NO 105 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCHPRVFLFPPKPKDTLMISRTPEVT |
| | | | CVVTGLRDEDPEVQFNWYVDGVEVHNAKTKPREEQFCGCYSVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 106 | | Nucleotide sequence of SEQ ID NO 105 | gaagtgcaactggttgaatctggcggaggactggttcagcctggoggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttctgctgtcaccccagagtgtttctgtt |
| | | | ccctccaaagcctaaggacaccctgatgatcagcagaacccctgaagtgacctgtgtcgtgaccggcctgagagatgaaga |
| | | | tcccgaggtgcagttcaattggtacgtggacggcgtggaagtgcacaacgccaagaccaagcctagagaggaacagttctg |
| | | | cggctgctacagcgtggtgtctgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 107 | 3560 | Protein sequence NO 107 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCHPSVFLFPPKPKDTLMISRTPEVT |
| | | | CVVVGLRQEDPEVQFNWYVDGVEVHNAKTKPREEQFCGCYRVVSVLTVLHQDWLNGKEYKCKV |
| | | | SNKGLPSSIEKTISKAKGQPREPQVYTLPPSQEEMTKNQVSLTCLVKGFYPSDIAVEWESNGQPENN |
| | | | YKTTPPVLDSDGSFFLYSRLTVDKSRWQEGNVFSCSVMHEALHNHYTQKSLSLSL |
| 108 | | Nucleotide sequence of SEQ ID NO 107 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttctgttgtcacccttccgtgtttctgttc |
| | | | cctccaaagcctaaggacaccctgatgatcagcagaacccctgaagtgacctgtgtggtcgtgggccttagacaagaggac |
| | | | cccgaggtgcagttcaattggtacgtggacggcgtggaagtgcacaacgccaagaccaagcctagagaggaacagttctgc |
| | | | ggctgctacagagtggtgtctgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 109 | 3561 | Protein sequence NO 109 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCGPSVFLFPPKPKDTLMISRTPEVT |
| | | | CVVVDVSQEDPEVQFNWYVDGVEVHNAKTKPREEQFCSCYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 110 | | Nucleotide sequence of SEQ ID NO 109 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttttgttgcggccctagcgtgttcctgttt |
| | | | cctccaaagcctaaggacaccctgatgatcagcagaacccctgaagtgacctgcgtggtggtggatgtgtctcaagaggacc |
| | | | ccgaggtgcagttcaattggtacgtggacggcgtggaagtgcacaacgccaagaccaagcctagaggaacagttctgct |
| | | | cctgctacagagtggtgtccgtgctgacagtgctgcaccaggactggctgaacggcaaagagtacaagtgcaaggtgtcca |
| | | | acaagggcctgccaagcagcatcgagaaaaccatcagcaaggccaagggccagcctagggaacctcaggtttacaccctg |
| | | | cctccaagccaagaggaaatgaccaagaaccaggtgtccctgacctgcctcgtgaagggcttctacccttccgatatcgccg |
| | | | tggaatgggagagcaatggccagcctgagaacaactacaagacaacccctcctgtgctggacagcgacggcagcttttttct |
| | | | gtattctcggctgaccgtggacaagagcagatggcaagagggcaacgtgttcagctgctctgtgatgcacgaggccctgcac |
| | | | aaccactacacccagaagtctctgtctctgagcctg |
| 111 | 3562 | Protein sequence NO 111 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCGPSVFLFPPKPKDTLMISRTPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 112 | | Nucleotide sequence of SEQ ID NO 111 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttttgttgcggccctagcgtgttcctgttt |
| | | | cctccaaagcctaaggacaccctgatgatcagcagaacccctgaagtgacctgcgtggtggtggatgtgtctcaagaggacc |
| | | | ccgaggtgcagttcaattggtacgtggacggcgtggaagtgcacaacgccaagaccaagcctagagaggaacagttcaac |
| | | | agcacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 113 | 3563 | Protein sequence NO 113 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLMISRTPEVT |
| | | | CVVVDVSQEDPEVQFNWYVDGVEVHNAKTKPREEQFCSCYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 114 | | Nucleotide sequence of SEQ ID NO 113 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttctgc |
| | | | tcctgctacagagtggtgtccgtgctgacagtgctgcaccaggactggctgaacggcaaagagtacaagtgcaaggtgtcca |
| | | | acaagggcctgccaagcagcatcgagaaaaccatcagcaaggccaagggccagcctagggaacctcaggtttacaccctg |
| | | | cctccaagccaagaggaaatgaccaagaaccaggtgtccctgacctgcctcgtgaagggcttctacccttccgatatcgccg |
| | | | tggaatgggagagcaatggccagcctgagaacaactacaagacaacccctcctgtgctggacagcgacggcagcttttttct |
| | | | gtattctcggctgaccgtggacaagagcagatggcaagagggcaacgtgttcagctgctctgtgatgcacgaggccctgcac |
| | | | aaccactacacccagaagtctctgtctctgagcctg |
| 115 | 3564 | Protein sequence NO 115 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLCGPSVFLFPPKPKDTLMISRTPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 116 | | Nucleotide sequence of SEQ ID NO 115 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctgtgcggccctagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 117 | 3565 | Protein sequence NO 117 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCGGPSVFLFPPKPKDTLMISRTPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 118 | | Nucleotide sequence of SEQ ID NO 117 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaattttgtggcggccctagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 119 | 3566 | Protein sequence NO 119 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLMISRTPEVT |
| | | | CVVVDVSQEDPEVQFNWYVDGVEVHNAKTKPREEQFNSCYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 120 | | Nucleotide sequence of SEQ ID NO 119 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttcaa |
| | | | cagctgctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 121 | 3567 | Protein sequence NO 121 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLMISRTPEVT |
| | | | CVVVDVSQEDPEVQFNWYVDGVEVHNAKTKPREEQFCSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 122 | | Nucleotide sequence of SEQ ID NO 121 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttctgc |
| | | | tccacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 123 | 3568 | Protein sequence NO 123 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCGGPSVFLFPPKPKDTLMISRTPEVT |
| | | | CVVVDVSQEDPEVQFNWYVDGVEVHNAKTKPREEQFCSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 124 | | Nucleotide sequence of SEQ ID NO 123 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaattttgtggcggccctagcgtgttcctgtt |
| | | | tcctccaaagcctaaggacaccctgatgatcagcagaacccctgaagtgacctgcgtggtggtggatgtgtctcaagaggac |
| | | | cccgaggtgcagttcaattggtacgtggacggcgtggaagtgcacaacgccaagaccaagcctagagaggaacagttctgc |
| | | | tccacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 125 | 3569 | Protein sequence NO 125 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLYITREPEVTC |
| | | | VVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 126 | | Nucleotide sequence of SEQ ID NO 125 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 127 | 3570 | Protein sequence NO 127 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPPCPAPEFLGGPSVFLFPPKPKDTLYITREPEVTC |
| | | | VVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 128 | | Nucleotide sequence of SEQ ID NO 127 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctccatgtcctgctccagaatttctcggcggacccagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 129 | 3571 | Protein sequence NO 129 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCHPRVFLFPPKPKDTLYITREPEVTC |
| | | | VVTGLRDEDPEVQFNWYVDGVEVHNAKTKPREEQFCGCYSVVSVLTVLHQDWLNGKEYKCKVSN |
| | | | KGLPSSIEKTISKAKGQPREPQVYTLPPSQEEMTKNQVSLTCLVKGFYPSDIAVEWESNGQPENNYK |
| | | | TTPPVLDSDGSFFLYSRLTVDKSRWQEGNVFSCSVMHEALHNHYTQKSLSLSL |
| 130 | | Nucleotide sequence of SEQ ID NO 129 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttctgctgtcaccccagagtgtttctgtt |
| | | | ccctccaaagcctaaggacacactgtacatcacccgcgagcctgaagtgacctgtgtcgtgacaggactgagagatgagga |
| | | | ccccgaggtgcagttcaattggtacgtggacggcgtggaagtgcacaacgccaagaccaagcctagagaggaacagttctg |
| | | | cggctgctacagcgtggtgtctgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 131 | 3572 | Protein sequence NO 131 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCHPSVFLFPPKPKDTLYITREPEVTC |
| | | | VVVGLRQEDPEVQFNWYVDGVEVHNAKTKPREEQFCGCYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 132 | | Nucleotide sequence of SEQ ID NO 131 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttctgttgtcacccttccgtgtttctgttc |
| | | | cctccaaagcctaaggacacactgtacatcacccgcgagcctgaagtgacatgtgtggtcgtgggactgagacaagaggac |
| | | | cccgaggtgcagttcaattggtacgtggacggcgtggaagtgcacaacgccaagaccaagcctagagaggaacagttctgc |
| | | | ggctgctacagagtggtgtctgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 133 | 3573 | Protein sequence NO 133 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCGPSVFLFPPKPKDTLYITREPEVTC |
| | | | VVVDVSQEDPEVQFNWYVDGVEVHNAKTKPREEQFCSCYRVVSVLTVLHQDWLNGKEYKCKVSN |
| | | | KGLPSSIEKTISKAKGQPREPQVYTLPPSQEEMTKNQVSLTCLVKGFYPSDIAVEWESNGQPENNYK |
| | | | TTPPVLDSDGSFFLYSRLTVDKSRWQEGNVFSCSVMHEALHNHYTQKSLSLSL |
| 134 | | Nucleotide sequence of SEQ ID NO 133 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttttgttgcggccctagcgtgttcctgttt |
| | | | cctccaaagcctaaggacacactgtacatcacccgcgagcctgaagtgacctgtgtggtggtggatgtgtcccaagaggacc |
| | | | ccgaggtgcagttcaattggtacgtggacggcgtggaagtgcacaacgccaagaccaagcctagagaggaacagttctgct |
| | | | cctgctacagagtggtgtccgtgctgacagtgctgcaccaggactggctgaacggcaaagagtacaagtgcaaggtgtcca |
| | | | acaagggcctgccaagcagcatcgagaaaaccatcagcaaggccaagggccagcctagggaacctcaggtttacaccctg |
| | | | cctccaagccaagaggaaatgaccaagaaccaggtgtccctgacctgcctcgtgaagggcttctacccttccgatatcgccg |
| | | | tggaatgggagagcaatggccagcctgagaacaactacaagacaacccctcctgtgctggacagcgacggcagcttttttct |
| | | | gtattctcggctgaccgtggacaagagcagatggcaagagggcaacgtgttcagctgctctgtgatgcacgaggccctgcac |
| | | | aaccactacacccagaagtctctgtctctgagcctg |
| 135 | 3574 | Protein sequence NO 135 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCCGPSVFLFPPKPKDTLYITREPEVTC |
| | | | VVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 136 | | Nucleotide sequence of SEQ ID NO 135 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgagttttgttgcggccctagcgtgttcctgttt |
| | | | cctccaaagcctaaggacacactgtacatcacccgcgagcctgaagtgacctgtgtggtggtggatgtgtcccaagaggacc |
| | | | ccgaggtgcagttcaattggtacgtggacggcgtggaagtgcacaacgccaagaccaagcctagagaggaacagttcaac |
| | | | agcacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 137 | 3575 | Protein sequence NO 137 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLYITREPEVTC |
| | | | VVVDVSQEDPEVQFNWYVDGVEVHNAKTKPREEQFCSCYRVVSVLTVLHQDWLNGKEYKCKVSN |
| | | | KGLPSSIEKTISKAKGQPREPQVYTLPPSQEEMTKNQVSLTCLVKGFYPSDIAVEWESNGQPENNYK |
| | | | TTPPVLDSDGSFFLYSRLTVDKSRWQEGNVFSCSVMHEALHNHYTQKSLSLSL |
| 138 | | Nucleotide sequence of SEQ ID NO 137 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttctgc |
| | | | tcctgctacagagtggtgtccgtgctgacagtgctgcaccaggactggctgaacggcaaagagtacaagtgcaaggtgtcca |
| | | | acaagggcctgccaagcagcatcgagaaaaccatcagcaaggccaagggccagcctagggaacctcaggtttacaccctg |
| | | | cctccaagccaagaggaaatgaccaagaaccaggtgtccctgacctgcctcgtgaagggcttctacccttccgatatcgccg |
| | | | tggaatgggagagcaatggccagcctgagaacaactacaagacaacccctcctgtgctggacagcgacggcagcttttttct |
| | | | gtattctcggctgaccgtggacaagagcagatggcaagagggcaacgtgttcagctgctctgtgatgcacgaggccctgcac |
| | | | aaccactacacccagaagtctctgtctctgagcctg |
| 139 | 3576 | Protein sequence NO 139 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLCGPSVFLFPPKPKDTLYITREPEVTC |
| | | | VVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 140 | | Nucleotide sequence of SEQ ID NO 139 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctgtgcggccctagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 141 | 3577 | Protein sequence NO 141 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCGGPSVFLFPPKPKDTLYITREPEVT |
| | | | CVVVDVSQEDPEVQFNWYVDGVEVHNAKTKPREEQFNSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 142 | | Nucleotide sequence of SEQ ID NO 141 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaattttgtggcggccctagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttcaa |
| | | | cagcacctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctcaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 143 | 3578 | Protein sequence NO 143 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLYITREPEVTC |
| | | | VVVDVSQEDPEVQFNWYVDGVEVHNAKTKPREEQFNSCYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 144 | | Nucleotide sequence of SEQ ID NO 143 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttcaa |
| | | | cagctgctacagagtggtgtccgtgctgacagtgctgcaccaggactggctgaacggcaaagagtacaagtgcaaggtgtc |
| | | | caacaagggcctgccaagcagcatcgagaaaaccatcagcaaggccaagggccagcctagggaacctcaggtttacaccc |
| | | | tgcctccaagccaagaggaaatgaccaagaaccaggtgtccctgacctgcctcgtgaagggcttctacccttccgatatcgc |
| | | | cgtggaatgggagagcaatggccagcctgagaacaactacaagacaacccctcctgtgctggacagcgacggcagcttttt |
| | | | tctgtattctcggctgaccgtggacaagagcagatggcaagagggcaacgtgttcagctgctctgtgatgcacgaggccctg |
| | | | cacaaccactacacccagaagtctctgtctctgagcctg |
| 145 | 3579 | Protein sequence NO 145 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFLGGPSVFLFPPKPKDTLYITREPEVTC |
| | | | VVVDVSQEDPEVQFNWYVDGVEVHNAKTKPREEQFCSTYRVVSVLTVLHQDWLNGKEYKCKVSN |
| | | | KGLPSSIEKTISKAKGQPREPQVYTLPPSQEEMTKNQVSLTCLVKGFYPSDIAVEWESNGQPENNYK |
| | | | TTPPVLDSDGSFFLYSRLTVDKSRWQEGNVFSCSVMHEALHNHYTQKSLSLSL |
| 146 | | Nucleotide sequence of SEQ ID NO 145 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaatttctcggcggacccagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttctgc |
| | | | tccacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 147 | 3580 | Protein sequence NO 147 | EVQLVESGGGLVQPGGSLKLSCAASGFTFNTYAMNWVRQASGKGLEWVGRIRSKYNNYATYYAD |
| | | | SVKDRFTISRDDSKSTLYLQMNSLKTEDTAVYYCVRHGNFGNSYVSWFAYWGQGTLVTVSSASTK |
| | | | GPSVFPLAPCSRSTSESTAALGCLVKDYFPEPVTVSWNSGALTSGVHTFPAVLQSSGLYSLSSVVTVP |
| | | | SSSLGTKTYTCNVDHKPSNTKVDKRVESKYGPPCPSCPAPEFCGGPSVFLFPPKPKDTLYITREPEVT |
| | | | CVVVDVSQEDPEVQFNWYVDGVEVHNAKTKPREEQFCSTYRVVSVLTVLHQDWLNGKEYKCKVS |
| | | | NKGLPSSIEKTISKAKGQPREPQVYTLPPSQEEMTKNQVSLTCLVKGFYPSDIAVEWESNGQPENNY |
| | | | KTTPPVLDSDGSFFLYSRLTVDKSRWQEGNVFSCSVMHEALHNHYTQKSLSLSL |
| 148 | | Nucleotide sequence of SEQ ID NO 147 | gaagtgcaactggttgaatctggcggaggactggttcagcctggcggatctctgaaactgtcttgtgccgccagcggcttcac |
| | | | cttcaacacctacgccatgaactgggtccgacaggcctctggcaaaggcctggaatgggtcggacggatcagaagcaagta |
| | | | caacaattacgccacctactacgccgacagcgtgaaggacagattcaccatcagccgggacgacagcaagagcaccctgt |
| | | | acctccagatgaacagcctgaaaaccgaggataccgccgtgtactattgcgtgcggcacggcaacttcggcaacagctatgt |
| | | | gtcttggtttgcctactggggccagggcacactggttacagtgtctagcgcctctacaaagggcccctccgtttttcctctggct |
| | | | ccttgttccagaagcaccagcgaatctacagccgctctgggctgcctggtcaaggattactttcctgagcctgtgaccgtgtcc |
| | | | tggaatagcggagcactgacaagcggcgtgcacacatttccagccgtgctgcaaagcagcggcctgtactctctgtctagcg |
| | | | tggtcacagtgcctagcagcagcctgggcaccaagacctacacctgtaacgtggaccacaagcctagcaataccaaggtgg |
| | | | acaagcgcgtggaatctaagtacggccctccttgtcctagctgccctgctcctgaattttgtggcggccctagcgtgttcctgtt |
| | | | tcctccaaagcctaaggacacactgtacatcacccgcgagcctgaagtgacctgtgtggtggtggatgtgtcccaagaggac |
| | | | cccgaggtgcagttcaattggtacgtggacggcgtggaagtgcacaacgccaagaccaagcctagagaggaacagttctgc |
| | | | tccacctacagagtggtgtccgtgctgacagtgctgcaccaggactggctgaacggcaaagagtacaagtgcaaggtgtcc |
| | | | aacaagggcctgccaagcagcatcgagaaaaccatcagcaaggccaagggccagcctagggaacctcaggtttacaccct |
| | | | gcctccaagccaagaggaaatgaccaagaaccaggtgtccctgacctgcctcgtgaagggcttctacccttccgatatcgcc |
| | | | gtggaatgggagagcaatggccagcctgagaacaactacaagacaacccctcctgtgctggacagcgacggcagctttttt |
| | | | ctgtattctcggctgaccgtggacaagagcagatggcaagagggcaacgtgttcagctgctctgtgatgcacgaggccctgc |
| | | | acaaccactacacccagaagtctctgtctctgagcctg |
| 149 | | Protein sequence NO 149 | RTVAAPSVFIFPPSDEQLKSGTASVVCLLNNFYPREAKVQWKVDNALQSGNSQESVTEQDSKDSTY |
| | | (Fc region) | SLSSTLTLSKADYEKHKVYACEVTHQGLSSPVTKSFNRGEC |
| 150 | | Nucleotide sequence of SEQ ID NO 149 | cggaccgtggccgcccccagcgtgttcatcttcccccccagcgacgagcagctgaagagcggcaccgccagcgtggtgtgcc |
| | | (Fc region) | tgctgaacaacttctacccccgggaggccaaggtgcagtggaaggtggacaacgccctgcagagcggcaacagccaggag |
| | | | agcgtgaccgagcaggacagcaaggacagcacctacagcctgagcagcaccctgaccctgagcaaggccgactacgaga |
| | | | agcacaaggtgtacgcctgcgaggtgacccaccagggcctgagcagccccgtgaccaagagcttcaaccggggcgagtgc |
| 151 | | Protein sequence NO 151 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELCCGPSVFLFPPKPKDTLMI |
| | | | SRTPEVTCVVVDVSHEDPEVKFNWYVDGVEVHNAKTKPREEQYNSTYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 152 | | Nucleotide sequence of SEQ ID NO 151 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgtgctgcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtacaacagcacctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 153 | | Protein sequence NO 153 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQYCSCYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 154 | | Nucleotide SEQ ID NO sequence of 153 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgggcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtactgcagctgctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 155 | | Protein sequence NO 155 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELLCGPSVFLFPPKPKDTLMI |
| | | | SRTPEVTCVVVDVSHEDPEVKFNWYVDGVEVHNAKTKPREEQYNSTYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 156 | | Nucleotide sequence of SEQ ID NO 155 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgtgcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtacaacagcacctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 157 | | Protein sequence NO 157 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELCGGPSVFLFPPKPKDTLM |
| | | | ISRTPEVTCVVVDVSHEDPEVKFNWYVDGVEVHNAKTKPREEQYNSTYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 158 | | Nucleotide sequence of SEQ ID NO 157 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgtgcggcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtacaacagcacctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 159 | | Protein sequence NO 159 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQYNSCYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 160 | | Nucleotide sequence of SEQ ID NO 159 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgggcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtacaacagctgctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 161 | | Protein sequence NO 161 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQYCSTYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 162 | | Nucleotide sequence of SEQ ID NO 161 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgggcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtactgcagcacctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 163 | | Protein sequence NO 163 | ASTKGPSVFPLAPSSKSTSGGTAALGCLVKDYFPEPVTVSWNGALTSGVHTFPAVLQSSGLYSLSSV |
| | | (Fc region) | VTVPSSSLGTQTYICNVNHKPSNTKVDKRVEPKSCDKTHTCPPCPAPELLGGPSVFLFPPKPKDTLMI |
| | | | SRTPEVTCVVVAVSHEDPEVKFNWYVDGVEVHNAKTKPREEQYASTYRVVSVLTVLHQDWLNGK |
| | | | EYKCKVSNKALAAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 164 | | Nucleotide sequence of SEQ ID NO 163 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgggcggccccagcgtgttcctgttcccccccaagcccaaggacaccctgatga |
| | | | tcagccggacccccgaggtgacctgcgtggtggtggccgtgagccacgaggaccccgaggtgaagttcaactggtacgtgg |
| | | | acggcgtggaggtgcacaacgccaagaccaagccccgggaggagcagtacgccagcacctaccgggtggtgagcgtgctg |
| | | | accgtgctgcaccaggactggctgaacggcaaggagtacaagtgcaaggtgagcaacaaggccctggccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |
| 165 | | Protein sequence NO 165 | ASTKGPSVFPLAPSSKSTSGGTAALGCLVKDYFPEPVTVSWNSGALTSGVHTFPAVLQSSGLYSLSSV |
| | | (Fc region) | VTVPSSSLGTQTYICNVNHKPSNTKVDKRVEPKSCDKTHTCPPCPAPELLGGPSVFLFPPKPKDTLMI |
| | | | SRTPEVTCVVVDVSHEDPEVKFNWYVDGVEVHNAKTKPREEQYNSTYRVVSVLTVLHQDWLNGK |
| | | | EYKCKVSNKALPAPIEKTISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALHNHYTQKSLSLSPGK |
| 166 | | Nucleotide sequence of SEQ ID NO 165 | gccagcaccaagggccccagcgtgttccccctggcccccagcagcaagagcaccagcggcggcaccgccgccctgggctgc |
| | | (Fc region) | ctggtgaaggactacttccccgagcccgtgaccgtgagctggaacagcggcgccctgaccagcggcgtgcacaccttccccg |
| | | | ccgtgctgcagagcagcggcctgtacagcctgagcagcgtggtgaccgtgcccagcagcagcctgggcacccagacctaca |
| | | | tctgcaacgtgaaccacaagcccagcaacaccaaggtggacaagcgggtggagcccaagagctgcgacaagacccacac |
| | | | ctgccccccctgccccgcccccgagctgctgggcggccccagcgtgttcctgttcccccccaagcccaaggacaccctgatga |
| | | | tcagccggacccccgaggtgacctgcgtggtggtggacgtgagccacgaggaccccgaggtgaagttcaactggtacgtgg |
| | | | acggcgtggaggtgcacaacgccaagaccaagccccgggaggagcagtacaacagcacctaccgggtggtgagcgtgctg |
| | | | accgtgctgcaccaggactggctgaacggcaaggagtacaagtgcaaggtgagcaacaaggccctgcccgcccccatcga |
| | | | gaagaccatcagcaaggccaagggccagccccgggagccccaggtgtacaccctgccccccagccgggaggagatgacc |
| | | | aagaaccaggtgagcctgacctgcctggtgaagggcttctaccccagcgacatcgccgtggagtgggagagcaacggcca |
| | | | gcccgagaacaactacaagaccaccccccccgtgctggacagcgacggcagcttcttcctgtacagcaagctgaccgtgga |
| | | | caagagccggtggcagcagggcaacgtgttcagctgcagcgtgatgcacgaggccctgcacaaccactacacccagaaga |
| | | | gcctgagcctgagccccggcaag |

TABLE 9
Biophysical properties of the engineered immunoglobulins and their binding affinities
to human Fc receptors as well as the Fc effector functions

| | | | Relative | | | | | | | | | |
|---|---|---|---|---|---|---|---|---|---|---|---|---|
| | | | Expression | | | | | | | | | |
| | | | yield after | | | | Maximum | | | | | |
| | | | 2-step | Aggre- | Maximum | Maximum | response at | | Maximum | EC50 | | |
| | | Mutations | purification | gation | response at | response | 300 nM | Maximum | response at | in | Maximum | CH2 |
| Anti-CD3 | | (according | (% of | after | 100 nM | for | hFcγR3a | response at | 1840 nM | RGA | response in | domain |
| monospecific | SEQ | to EU | WT final | capture | hFcγR1a | hFcγR2a | F158V | 20 nM | hFcRn | assay | RGA assay | TM |
| hIgG1 | ID | numbering) | yield) | (%) | (RU) | (RU) | (RU) | hC1q (RU) | (RU) | (M) | (LU) | (° C.) |
| CD3_WT | 1 | — | 100.0 | 14.8 | 100 | Nd | 100 | 100 | 100 | 22.0 | 44534 | 70.0 |
| (HEK-293T- | 3 | — | | | | | | | | | | |
| 17SF) | | | | | | | | | | | | |
| CD3_WT_YTE | 5 | M252Y, | 102.7 | 13.7 | 97 | Nd | 71 | Nd | 586 | 16.9 | 39880 | 64.0 |
| (HEK-293T- | | S254T, | | | | | | | | | | |
| 17SF) | | T256E | | | | | | | | | | |
| | 3 | — | | | | | | | | | | |
| CD3_1 | 7 | L235C, | 96.1 | 14.8 | 2 | Nd | 10 | 5 | Nd | 556.1 | 24741 | 71.0 |
| (HEK-293T- | | G236C, | | | | | | | | | | |
| 17SF) | | G237H, | | | | | | | | | | |
| | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C | | | | | | | | | | |
| | | R301S | | | | | | | | | | |
| | 3 | — | | | | | | | | | | |
| CD3_1_YTE | 9 | L235C | 97.6 | 16.0 | 6 | Nd | 20 | Nd | 290 | 656.8 | 12014 | 71.0 |
| (HEK-293T- | | G236C, | | | | | | | | | | |
| 17SF) | | G237H | | | | | | | | | | |
| | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C | | | | | | | | | | |
| | | R301S, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| CD3_2 | 11 | L235C | 96.1 | 14.0 | 5 | Nd | 20 | 7 | Nd | 448.2 | 25456 | 71.0 |
| (HEK-293T- | | G236C, | | | | | | | | | | |
| 17SF) | | G237H, | | | | | | | | | | |
| | | D265G, | | | | | | | | | | |
| | | V266L, | | | | | | | | | | |
| | | S267R, | | | | | | | | | | |
| | | N297C, | | | | | | | | | | |
| | | S298G, | | | | | | | | | | |
| | | T299C | | | | | | | | | | |
| | 3 | — | | | | | | | | | | |
| CD3_2_YTE | 13 | L235C, | 90.0 | 13.9 | 3 | Nd | 0 | Nd | 340 | 417.9 | 11900 | 71.0 |
| (HEK-293T- | | G236C, | | | | | | | | | | |
| 17SF) | | G237H, | | | | | | | | | | |
| | | D265G, | | | | | | | | | | |
| | | V266L, | | | | | | | | | | |
| | | S267R, | | | | | | | | | | |
| | | N297C, | | | | | | | | | | |
| | | S298G, | | | | | | | | | | |
| | | T299C, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| CD3_3 | 15 | L235C, | 104.7 | 14.4 | 3 | Nd | 2 | 7 | Nd | 560,3 | 23248 | Nd |
| (HEK-293T- | | G236C. | | | | | | | | | | |
| 17SF) | | G237H, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C | | | | | | | | | | |
| | 3 | — | | | | | | | | | | |
| CD3_3_YTE | 17 | L235C | 102.7 | 14.2 | 7 | Nd | 10 | Nd | Nd | 591.8 | 13100 | Nd |
| (HEK-293T- | | G236C | | | | | | | | | | |
| 17SF) | | G237H, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C, | | | | | | | | | | |
| | | M252Y, | | | | | | | | | | |
| | | S254T | | | | | | | | | | |
| | | T256E | | | | | | | | | | |
| | 3 | — | | | | | | | | | | |
| CD3_4 | 19 | L235C, | 53.7 | 7.9 | 4 | Nd | 10 | 9 | Nd | 471.5 | 24908 | Nd |
| (HEK-293T- | | G236C, | | | | | | | | | | |
| 17SF) | | G237H, | | | | | | | | | | |
| | | N297C, | | | | | | | | | | |
| | | S298G, | | | | | | | | | | |
| | | T299C | | | | | | | | | | |
| | 3 | — | | | | | | | | | | |
| CD3_4_YTE | 21 | L235C, | 70.1 | 9.8 | 4 | Nd | 10 | Nd | Nd | 520.0 | 16496 | Nd |
| (HEK-293T- | | G236C, | | | | | | | | | | |
| 17SF) | | G237H, | | | | | | | | | | |
| | | N297C, | | | | | | | | | | |
| | | S298G, | | | | | | | | | | |
| | | T299C, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| CD3_5 | 23 | L235C, | 73.8 | 16.7 | 7 | Nd | 22 | 0 | Nd | 403.3 | 19851 | 70.5 |
| (HEK-293T- | | G236C, | | | | | | | | | | |
| 17SF) | | N297C, | | | | | | | | | | |
| | | T299C | | | | | | | | | | |
| | 3 | — | | | | | | | | | | |
| CD3_5_YTE | 25 | L235C, | 64.5 | 15.8 | 7 | Nd | 17 | Nd | 340 | 432.2 | 13116 | 69.5 |
| (HEK-293T- | | G236C, | | | | | | | | | | |
| 17SF) | | N297C, | | | | | | | | | | |
| | | T299C, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| CD3_6 | 27 | L235C, | 109.1 | 15.7 | 13 | Nd | 17 | 33 | 136 | 374.0 | 23600 | 70.5 |
| (HEK-293T- | | G236C | | | | | | | | | | |
| 17SF) | 3 | — | | | | | | | | | | |
| CD3_6_YTE | 29 | L235C, | 95.1 | 18.2 | 10 | Nd | 15 | Nd | 720 | 333.7 | 17394 | 69.5 |
| (HEK-293T- | | G236C, | | | | | | | | | | |
| 17SF) | | M252Y, | | | | | | | | | | |
| | | S254T, | | | | | | | | | | |
| | | T256E | | | | | | | | | | |
| | 3 | — | | | | | | | | | | |
| CD3_7 | 31 | N297C, | 70.5 | 18.0 | 21 | Nd | 5 | 2 | 80 | 411.6 | 25335 | 69.5 |
| (HEK-293T- | | T299C | | | | | | | | | | |
| 17SF) | 3 | — | | | | | | | | | | |
| CD3_7_YTE | 33 | N297C, | 67.1 | 14.4 | 31 | Nd | 19 | Nd | 284 | 68.3 | 26433 | 60.0 |
| (HEK-293T- | | T299C, | | | | | | | | | | |
| 17SF) | | M252Y, | | | | | | | | | | |
| | | S254T, | | | | | | | | | | |
| | | T256E | | | | | | | | | | |
| | 3 | — | | | | | | | | | | |
| CD3_8 | 35 | G236C | 87.1 | 13.2 | 2 | Nd | Nd | 1 | 126 | 269.9 | 11421 | 70.0 |
| (HEK-293T- | | | | | | | | | | | | |
| 17SF) | 3 | — | | | | | | | | | | |
| CD3_8_YTE | 39 | G236C, | 96.8 | 11.8 | 2 | Nd | Nd | 0 | 604 | 215.2 | 7593 | 64.0 |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| CD3_9 | 37 | L235C | 101.6 | 12.5 | 2 | Nd | Nd | 10 | 122 | 202.1 | 7892 | 71.0 |
| (HEK-293T- | | | | | | | | | | | | |
| 17SF) | 3 | — | | | | | | | | | | |
| CD3_9_YTE | 41 | L235C, | 96.8 | 12,9 | 2 | Nd | Nd | 4 | 696 | 264.9 | 7211 | 69.0 |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| CD3_10 | 43 | T299C | 103.2 | 14.9 | 18 | Nd | Nd | 3 | 126 | 279.8 | 15051 | 65.0 |
| (HEK-293T- | 3 | — | | | | | | | | | | |
| 17SF) | | | | | | | | | | | | |
| CD3_10_YTE | 47 | T299C, | 104.8 | 17.6 | 9 | Nd | Nd | 0 | 280 | 258.5 | 19876 | 57.0 |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| CD3_11 | 45 | N297C | 96.8 | 17.1 | 19 | Nd | Nd | 0 | 92 | 131.7 | 20526 | 66.0 |
| (HEK-293T- | | | | | | | | | | | | |
| 17SF) | 3 | — | | | | | | | | | | |
| CD3_11_YTE | 49 | N297C, | 103.2 | 16.6 | 12 | Nd | Nd | 1 | 230 | 73.8 | 23777 | 69.0 |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| CD3_12 | 51 | L235C, | 69.1 | Nd | Nd | Nd | Nd | Nd | Nd | 663.3 | 11553 | 69.5 |
| (HEK-293T- | | N297C | | | | | | | | | | |
| 17SF) | 3 | — | | | | | | | | | | |
| CD3_12_YTE | 53 | L235C, | 63.3 | Nd | Nd | Nd | Nd | Nd | Nd | 563.3 | 8959 | 57.0 |
| 17SF) | | N297C, | | | | | | | | | | |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| CD3_DANAPA | 55 | D265A, | 80.0 | 9.8 | 6 | Nd | 10 | 1 | Nd | 353.4 | 12761 | Nd |
| (HEK-293T- | | N297A, | | | | | | | | | | |
| 17SF) | | P329A | | | | | | | | | | |
| | 3 | — | | | | | | | | | | |
| CD3_DANAPA_ | 57 | D265A, | 67.6 | 9.1 | Nd | Nd | Nd | Nd | Nd | 600.2 | 12315 | 52.0 |
| YTE | | N297A, | | | | | | | | | | |
| (HEK-293T- | | P329A, | | | | | | | | | | |
| 17SF) | | M252Y, | | | | | | | | | | |
| | | S254T, | | | | | | | | | | |
| | | T256E | | | | | | | | | | |
| | 3 | — | | | | | | | | | | |
| | | | Relative | | | | | | | | | |
| | | | Expression | | | | | | | | | |
| | | | yield after | | | Maximum | Maximum | | | | | |
| | | | 2-step | Aggre- | Maximum | response | response at | | | | | |
| | | Mutations | purification | gation | response at | at | 1000 nM | Maximum | KD for | | | CH2 |
| Anti-TargetC | | (according | (% of | after | 20 nM | 4000 nM | hFcγR3a | response at | hFcRn | | | domain |
| monospecific | SEQ | to EU | WT final | capture | hFcγR1a | hFcγR2a | F158V | 200 nM | (nM) at pH | | | TM |
| hIgG1 | ID | numbering | yield) | (%) | (RU) | (RU) | (RU) | hC1q (RU) | 5.8 | | | (° C.) |
| TargetC_WT | 165 | — | 100.0 | 1.1 | 100 | 100 | 100 | 100 | 1963 | | | 66.0 |
| (CHO- | 149 | — | | | | | | | | | | |
| C8TD) | | | | | | | | | | | | |
| TargetC_6 | 151 | L235C | 100.0 | 0.7 | 0 | 0 | 0 | 0 | 1711 | | | 69.0 |
| (CHO- | | G236C | | | | | | | | | | |
| C8TD) | 149 | — | | | | | | | | | | |
| TargetC_7 | 153 | N297C, | 104.2 | 2.3 | 18 | 0 | 0 | 0 | 2239 | | | 62.0 |
| (CHO- | | T299C | | | | | | | | | | |
| C8TD) | 149 | — | | | | | | | | | | |
| TargetC_8 | 155 | G236C | 95.8 | 0.8 | 0 | 0 | 0 | 0 | 1814 | | | 67.0 |
| (CHO- | | | | | | | | | | | | |
| C8TD) | 149 | — | | | | | | | | | | |
| TargetC_9 | 157 | L235C | 100.0 | 0.5 | 0 | 0 | 0 | 4.4 | 1822 | | | 68.0 |
| (CHO- | 149 | — | | | | | | | | | | |
| C8TD) | | | | | | | | | | | | |
| TargetC_10 | 159 | T299C | 112.5 | 2.2 | 11 | 0 | 0 | 0 | 2200 | | | 60.0 |
| (CHO- | 149 | — | | | | | | | | | | |
| C8TD) | | | | | | | | | | | | |
| TargetC_11 | 161 | N297C | 108.3 | 6.6 | 27 | 0 | 0 | 2.6 | 2270 | | | 58.0 |
| (CHO- | | | | | | | | | | | | |
| C8TD) | 149 | — | | | | | | | | | | |
| TargetC_DA | 163 | D265A, | 95.8 | 2.8 | 0 | 0 | 0 | 0 | Nd | | | 57.0 |
| NAPA | | N297A, | | | | | | | | | | |
| (CHO- | | P329A | | | | | | | | | | |
| C8TD) | 149 | — | | | | | | | | | | |
| | | | Relative | | | | | | | | | |
| | | | Expression | | | | | | | | | |
| | | | yield after | | | | Maximum | | | | | |
| Anti- | | | 2-step | Aggre- | Maximum | Maximum | response at | | Maximum | EC50 | | |
| CD3xTargetA: | | Mutations | purification | gation | response at | response | 300 nM | Maximum | response at | in | Maximum | CH2 |
| Target B | | (according | (% of | after | 100 nM | for | hFcγR3a | response at | 1840 nM | RGA | response in | domain |
| trispecific | SEQ | to EU | WT final | capture | hFcγR1a | hFcγR2a | F158V | 200 nM | hFcRn | assay | RGA assay | TM |
| hIgG1 | ID | numbering) | yield) | (%) | (RU) | (RU) | (RU) | hC1q (RU) | (RU) | (M) | (LU) | (° C.) |
| CD3xTarget | 59 | Y349C, | 100.0 | 10.8 | Nd | Nd | Nd | Nd | Nd | 0.1 | 38754 | 59.5 |
| AxTargetB | | T366S, | | | | | | | | | | |
| WT | | L368A, | | | | | | | | | | |
| (HEK-293T- | | Y407V | | | | | | | | | | |
| 17SF) | 75 | S354C | | | | | | | | | | |
| | | T366W | | | | | | | | | | |
| | 91 | — | | | | | | | | | | |
| CD3xTarget | 61 | D265A, | 53.5 | 22.7 | Nd | Nd | Nd | Nd | Nd | 13.4 | 29755 | 56.0 |
| AxTargetB_ | | N297A, | | | | | | | | | | |
| DANAPA | | P329A, | | | | | | | | | | |
| (HEK-293T- | | Y349C, | | | | | | | | | | |
| 17SF) | | T366S, | | | | | | | | | | |
| | | L368A, | | | | | | | | | | |
| | | Y407V | | | | | | | | | | |
| | 77 | D265A, | | | | | | | | | | |
| | | N297A, | | | | | | | | | | |
| | | P329A, | | | | | | | | | | |
| | | S354C, | | | | | | | | | | |
| | | T366W | | | | | | | | | | |
| | 91 | — | | | | | | | | | | |
| CD3xTarget | 63 | L235C | 86.8 | 15.4 | Nd | Nd | Nd | Nd | Nd | 52.7 | 20996 | 61.0 |
| AxTargetB_1 | | G236C, | | | | | | | | | | |
| (HEK-293T- | | G237H, | | | | | | | | | | |
| | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C, | | | | | | | | | | |
| | | R301S, | | | | | | | | | | |
| | | Y349C, | | | | | | | | | | |
| | | T366S, | | | | | | | | | | |
| | | L368A, | | | | | | | | | | |
| | | Y407V | | | | | | | | | | |
| | 79 | L235C | | | | | | | | | | |
| | | G236C | | | | | | | | | | |
| | | G237H, | | | | | | | | | | |
| | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C, | | | | | | | | | | |
| | | R301S, | | | | | | | | | | |
| | | S354C, | | | | | | | | | | |
| | | T366W | | | | | | | | | | |
| | 91 | — | | | | | | | | | | |
| CD3xTarget | 65 | L235C, | 66.7 | 13.5 | Nd | Nd | Nd | Nd | Nd | 108.6 | 18935 | 61.0 |
| AxTargetB_2 | | G236C, | | | | | | | | | | |
| (HEK-293T- | | G237H, | | | | | | | | | | |
| 17SF) | | D265G, | | | | | | | | | | |
| | | V266L, | | | | | | | | | | |
| | | S267R, | | | | | | | | | | |
| | | N297C, | | | | | | | | | | |
| | | S298G, | | | | | | | | | | |
| | | T299C, | | | | | | | | | | |
| | | Y349C, | | | | | | | | | | |
| | | T366S, | | | | | | | | | | |
| | | L368A | | | | | | | | | | |
| | | Y407V | | | | | | | | | | |
| | 81 | L235C, | | | | | | | | | | |
| | | G236C | | | | | | | | | | |
| | | G237H, | | | | | | | | | | |
| | | D265G, | | | | | | | | | | |
| | | V266L, | | | | | | | | | | |
| | | S267R, | | | | | | | | | | |
| | | N297C, | | | | | | | | | | |
| | | S298G, | | | | | | | | | | |
| | | T299C, | | | | | | | | | | |
| | | S354C, | | | | | | | | | | |
| | | T366W | | | | | | | | | | |
| | 91 | — | | | | | | | | | | |
| CD3xTarget | 67 | L235C | 117.5 | 10.0 | Nd | Nd | Nd | Nd | Nd | 21.2 | 23942 | 59.0 |
| AxTargetB_6 | | G236C, | | | | | | | | | | |
| (HEK-293T- | | Y349C, | | | | | | | | | | |
| 17SF) | | T366S, | | | | | | | | | | |
| | | L368A, | | | | | | | | | | |
| | | Y407V | | | | | | | | | | |
| | 83 | L235C, | | | | | | | | | | |
| | | G236C, | | | | | | | | | | |
| | | S354C, | | | | | | | | | | |
| | | T366W | | | | | | | | | | |
| | 91 | — | | | | | | | | | | |
| CD3xTarget | 69 | N297C, | 82.0 | 12.6 | Nd | Nd | Nd | Nd | Nd | 309.7 | 13041 | 59.0 |
| AxTargetB_7 | | T299C, | | | | | | | | | | |
| (HEK-293T- | | Y349C | | | | | | | | | | |
| 17SF) | | T366S, | | | | | | | | | | |
| | | L368A, | | | | | | | | | | |
| | | Y407V | | | | | | | | | | |
| | 85 | N297C, | | | | | | | | | | |
| | | T299C, | | | | | | | | | | |
| | | S354C, | | | | | | | | | | |
| | | T366W | | | | | | | | | | |
| | 183 | — | | | | | | | | | | |
| CD3xTarget | 71 | G236C, | 97.4 | 13.2 | Nd | Nd | Nd | Nd | Nd | 129.4 | 14807 | 59.0 |
| AxTargetB_8 | | Y349C, | | | | | | | | | | |
| (HEK-293T- | | T366S, | | | | | | | | | | |
| 17SF) | | L368A, | | | | | | | | | | |
| | | Y407V | | | | | | | | | | |
| | 87 | G236C, | | | | | | | | | | |
| | | S354C, | | | | | | | | | | |
| | | T366W | | | | | | | | | | |
| | 91 | — | | | | | | | | | | |
| CD3xTarget | 73 | L235C, | 115.4 | 9.7 | Nd | Nd | Nd | Nd | Nd | 246.0 | 4501 | 59.0 |
| AxTargetB_9 | | Y349C, | | | | | | | | | | |
| (HEK-293T- | | T366S, | | | | | | | | | | |
| 17SF) | | L368A, | | | | | | | | | | |
| | | Y407V | | | | | | | | | | |
| | 89 | L235C, | | | | | | | | | | |
| | | S354C, | | | | | | | | | | |
| | | T366W | | | | | | | | | | |
| | 91 | — | | | | | | | | | | |
| | | | Relative | | | | | | | | | |
| | | | Expression | | | | | | | | | |
| | | | yield after | | | | Maximum | | | | | |
| | | | 2-step | Aggre- | Maximum | Maximum | response at | | Maximum | EC50 | | |
| | | Mutations | purification | gation | response at | response | 300 nM | Maximum | response at | in | Maximum | CH2 |
| Anti-CD3 | | (according | (% of | after | 100 nM | for | hFcγR3a | response at | 1840 nM | RGA | response in | domain |
| monospecific | SEQ | to EU | WT final | capture | hFcγR1a | hFcγR2a | F158V | 20 nM | hFcRn | assay | RGA assay | TM |
| hIgG4 | ID | numbering) | yield) | (%) | (RU) | (RU) | (RU) | hC1q (RU) | (RU) | (M) | (LU) | (° C.) |
| IgG4 CD3 | 101 | — | 100.0 | 6.0 | Nd | Nd | Nd | Nd | Nd | 33.2 | 36059 | 70.0 |
| WT | | | | | | | | | | | | |
| (HEK-293T- | 3 | — | | | | | | | | | | |
| 17SF) | | | | | | | | | | | | |
| IgG4 CD3 | 125 | M252Y, | 65.1 | 6.0 | Nd | Nd | Nd | Nd | Nd | 29.8 | 31791 | 61.0 |
| WT_YTE | | S254T, | | | | | | | | | | |
| (HEK-293T- | | T256E | | | | | | | | | | |
| 17SF) | 3 | — | | | | | | | | | | |
| IgG4_CD3_S228P | 103 | S228P | 103.8 | 4.3 | Nd | No | Nd | Nd | Nd | 23.0 | 33960 | 70.0 |
| (HEK-293T- | 3 | — | | | | | | | | | | |
| 17SF) | | | | | | | | | | | | |
| IgG4_CD3_S | 127 | S228P, | 79.2 | 5.5 | Nd | Nd | Nd | Nd | Nd | 33.0 | 34895 | 61.0 |
| 228P YTE | | M252Y, | | | | | | | | | | |
| (HEK-293T- | | S254T, | | | | | | | | | | |
| 17SF) | | T256E | | | | | | | | | | |
| | 3 | — | | | | | | | | | | |
| IgG4_CD3_1 | 105 | L235C, | Nd | Nd | Nd | Nd | Nd | Nd | Nd | Nd | Nd | Nd |
| (HEK-293T- | | G236C. | | | | | | | | | | |
| 17SF) | | G237H, | | | | | | | | | | |
| | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C, | | | | | | | | | | |
| | | R301S | | | | | | | | | | |
| | 3 | — | | | | | | | | | | |
| IgG4_CD3_1_ | 129 | L235C, | Nd | Nd | Nd | Nd | Nd | Nd | Nd | Nd | Nd | Nd |
| YTE | | G236C | | | | | | | | | | |
| (HEK-293T- | | G237H, | | | | | | | | | | |
| 17SF) | | S239R, | | | | | | | | | | |
| | | V264T, | | | | | | | | | | |
| | | D265G, | | | | | | | | | | |
| | | V266L, | | | | | | | | | | |
| | | S267R, | | | | | | | | | | |
| | | H268D, | | | | | | | | | | |
| | | N297C, | | | | | | | | | | |
| | | S298G, | | | | | | | | | | |
| | | T299C, | | | | | | | | | | |
| | | R301S, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| IgG4_CD3 2 | 107 | L235C, | Nd | Nd | Nd | Nd | Nd | Nd | Nd | Nd | Nd | Nd |
| (HEK-293T- | | G236C, | | | | | | | | | | |
| 17SF) | | G237H, | | | | | | | | | | |
| | | D265G, | | | | | | | | | | |
| | | V266L, | | | | | | | | | | |
| | | S267R, | | | | | | | | | | |
| | | N297C | | | | | | | | | | |
| | | S298G, | | | | | | | | | | |
| | | T299C | | | | | | | | | | |
| | 3 | — | | | | | | | | | | |
| IgG4_CD3_2_ | 131 | L235C, | Nd | Nd | Nd | Nd | Nd | Nd | Nd | Nd | Nd | Nd |
| YTE | | G236C, | | | | | | | | | | |
| (HEK-293T- | | G237H, | | | | | | | | | | |
| 17SF) | | D265G, | | | | | | | | | | |
| | | V266L, | | | | | | | | | | |
| | | S267R, | | | | | | | | | | |
| | | N297C, | | | | | | | | | | |
| | | S298G, | | | | | | | | | | |
| | | T299C, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| IgG4_CD3_5 | 109 | L235C, | No | Nd | Nd | Nd | Nd | Nd | Nd | Nd | Nd | Nd |
| (HEK-293T- | | G236C, | | | | | | | | | | |
| 17SF) | | N297C, | | | | | | | | | | |
| | | T299C | | | | | | | | | | |
| | 3 | — | | | | | | | | | | |
| IgG4_CD3_5_ | 133 | L235C, | Nd | Nd | Nd | Nd | Nd | Nd | Nd | Nd | Nd | Nd |
| YTE | | G236C, | | | | | | | | | | |
| (HEK-293T- | | N297C, | | | | | | | | | | |
| 17SF) | | T299C, | | | | | | | | | | |
| | | M252Y, | | | | | | | | | | |
| | | S254T, | | | | | | | | | | |
| | | T256E | | | | | | | | | | |
| | 3 | — | | | | | | | | | | |
| IgG4_CD3_6 | 111 | L235C, | 89.6 | 6.5 | Nd | Nd | Nd | Nd | Nd | 351.7 | 12035 | 70.0 |
| (HEK-293T- | | G236C | | | | | | | | | | |
| 17SF) | 3 | — | | | | | | | | | | |
| IgG4_CD3_6_ | 135 | L235C, | 68.9 | 6.8 | Nd | Nd | Nd | Nd | Nd | 257.4 | 7914 | 65.0 |
| YTE | | G236C, | | | | | | | | | | |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| IgG4_CD3_7 | 113 | N297C, | Nd | Nd | Nd | Nd | Nd | Nd | Nd | Nd | Nd | Nd |
| (HEK-293T- | | T299C | | | | | | | | | | |
| 17SF) | 3 | — | | | | | | | | | | |
| IgG4_CD3_7- | 137 | N297C, | Nd | Nd | Nd | Nd | Nd | Nd | Nd | Nd | Nd | Nd |
| YTE | | T299C, | | | | | | | | | | |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |
| IgG4_CD3_8 | 115 | G236C | 94.3 | 4.5 | Nd | Nd | Nd | Nd | Nd | 209.0 | 7043 | 70.0 |
| (HEK-293T- | | | | | | | | | | | | |
| 17SF) | 3 | — | | | | | | | | | | |
| IgG4_CD3_8_ | 139 | G236C, | 106.6 | 5.2 | Nd | Nd | Nd | Nd | Nd | 233.2 | 6452 | 62.0 |
| YTE | | M252Y, | | | | | | | | | | |
| (HEK-293T- | | S254T, | | | | | | | | | | |
| 17SF) | | T256E | | | | | | | | | | |
| | 3 | — | | | | | | | | | | |
| IgG4_CD3 9 | 117 | L235C | 102.8 | 5.0 | Nd | Nd | Nd | Nd | Nd | 217.4 | 5652 | 70.0 |
| (HEK-293T- | | | | | | | | | | | | |
| 17SF) | 3 | | | | | | | | | | | |
| IgG4_CD3_9_ | 141 | L235C | 88.7 | 5.3 | Nd | Nd | Nd | Nd | Nd | 228.9 | 5612 | 64.0 |
| YTE | | M252Y, | | | | | | | | | | |
| (HEK-293T- | | S254T, | | | | | | | | | | |
| 17SF) | | T256E | | | | | | | | | | |
| | 3 | — | | | | | | | | | | |
| IgG4_CD3_10 | 119 | T299C | Nd | Nd | Nd | Nd | Nd | Nd | Nd | Nd | Nd | Nd |
| (HEK-293T- | 3 | — | | | | | | | | | | |
| 17SF) | | | | | | | | | | | | |
| IgG4_CD3_10_ | 143 | T299C, | Nd | Nd | Nd | Nd | Nd | Nd | Nd | Nd | Nd | Nd |
| YTE | | M252Y, | | | | | | | | | | |
| (HEK-293T- | | S254T, | | | | | | | | | | |
| 17SF) | | T256E | | | | | | | | | | |
| | 3 | — | | | | | | | | | | |
| IgG4_CD3_11 | 121 | N297C | Nd | Nd | Nd | Nd | Nd | Nd | Nd | Nd | Nd | Nd |
| (HEK-293T- | 3 | — | | | | | | | | | | |
| 17SF) | | | | | | | | | | | | |
| IgG4_CD3_11_ | 145 | N297C, | Nd | Nd | Nd | Nd | Nd | Nd | Nd | Nd | Nd | Nd |
| YTE | | M252Y. | | | | | | | | | | |
| (HEK-293T- | | S254T, | | | | | | | | | | |
| 17SF) | | T256E | | | | | | | | | | |
| | 3 | — | | | | | | | | | | |
| IgG4_CD3_12_ | 123 | L235C, | Nd | Nd | Nd | Nd | Nd | Nd | Nd | Nd | Nd | Nd |
| (HEK-293T- | | N297C | | | | | | | | | | |
| 17SF) | 3 | — | | | | | | | | | | |
| IgG4_CD3_12_ | 147 | L235C, | Nd | Nd | Nd | Nd | Nd | Nd | Nd | Nd | Nd | Nd |
| YTE | | N297C, | | | | | | | | | | |
| (HEK-293T- | | M252Y, | | | | | | | | | | |
| 17SF) | | S254T, | | | | | | | | | | |
| | | T256E | | | | | | | | | | |
| | 3 | — | | | | | | | | | | |

1. An engineered immunoglobulin or fragment thereof comprising a Fc variant of a wild-type human IgG Fc polypeptide and one or more antigen binding domains, wherein the Fc variant exhibits reduced effector functions as compared to the wild-type human IgG Fc polypeptide, and wherein the Fc variant comprises a cysteine substitution at position: 235, and wherein the amino acid residue is numbered according to the EU numbering.

2. (canceled)

3. The engineered immunoglobulin or fragment thereof of claim 1, wherein the engineered immunoglobulin or fragment thereof further comprises: one or more amino acid substitutions in the Fc variant which enhance the half-life of the engineered immunoglobulin or fragment thereof via enhanced FcRn binding and/or one or more amino acid substitutions that facilitate correct chain pairing of two different Fc chains.

4. The engineered immunoglobulin or fragment thereof of claim 3, wherein the half-life extending/FcRn binding enhancing amino acid substitutions are selected from the group consisting of mutation sets: M252Y/S254T/T256E (YTE), M428L/N434S (LS) and T250Q/M428L(QL) and T307Q/N434A(QA).

5. The engineered immunoglobulin or fragment thereof of claim 4, wherein the half-life extending/FcRn binding enhancing amino acid substitution is M252Y/S254T/T256E (YTE).

6. The engineered immunoglobulin or fragment thereof according to claim 1, wherein the engineered immunoglobulin or fragment thereof is a human IgG1, IgG2, IgG3 or IgG4 antibody.

7. The engineered immunoglobulin or fragment thereof according to claim 1, wherein the engineered immunoglobulin or fragment thereof is a human IgG1 antibody.

8. The engineered immunoglobulin or fragment thereof according to claim 1, wherein the engineered immunoglobulin or fragment thereof is a multispecific binding molecule, which comprises chain pairing amino acid substitutions selected from the group consisting of knob-into-hole (KiH), SEEDbody, RF-mutation, DEKK-mutation, electrostatic steering mutations and Fab-arm exchange.

9. The engineered immunoglobulin or fragment thereof of claim 8, wherein the chain paring amino acid substitutions are knob-into-hole (KiH) mutations, wherein the multispecific binding molecule comprises a first constant heavy chain with amino acid substitutions of S354C and T366W and a second constant heavy chain with amino acid substitutions of Y349C, T366S, L368A and Y407V, and wherein the amino acid residues are numbered according to the EU numbering.

10. The engineered immunoglobulin or fragment thereof of claim 9, wherein the multispecific binding molecule further comprises M252Y/S254T/T256E (YTE).

11. A pharmaceutical composition comprising the engineered immunoglobulin or fragment thereof according to claim 1, in combination with one or more pharmaceutically acceptable excipients, diluents or carriers.

12. The pharmaceutical composition according to claim 11, further comprising one or more additional active agents.

13. An isolated nucleic acid molecule encoding the engineered immunoglobulin or fragment thereof according to claim 1.

14. A cloning or expression vector comprising the isolated nucleic acid molecule encoding the engineered immunoglobulin or fragment thereof of claim 1, wherein the vector is suitable for the recombinant production of the engineered immunoglobulin or fragment thereof according to claim 1.

15. A recombinant host cell comprising one or more cloning or expression vectors according to claim 14.

16. A method of preparing the engineered immunoglobulin or fragment thereof according to claim 1, the method comprising culturing a host cell, purifying and recovering the engineered immunoglobulin or fragment thereof from the host cell culture, and formulating the engineered immunoglobulin or fragment thereof in a pharmaceutically acceptable composition, wherein the host cell comprises one or more cloning or expression vectors, wherein the cloning or expression vector comprises the isolated nucleic acid molecule encoding the engineered immunoglobulin or fragment thereof of claim 1; and the vector is suitable for the recombinant production of the engineered immunoglobulin or fragment thereof according to claim 1.

17. An engineered immunoglobulin or fragment thereof according to claim 1 for use as a medicament.
